(19) United States
(12) Patent Application Publication
(10) Pub. No.: US 2015/0246024 A1
(43) Pub. Date: Sep. 3, 2015

(54) METHODS AND COMPOSITIONS FOR INHIBITING GRAM POSITIVE BACTERIA

(71) Applicant: The University of Chicago, Chicago, IL (US)

(72) Inventors: Stefan Richter, Chicago, IL (US); Dominique M. Missiakas, Chicago, IL (US); Olaf Schneewind, Chicago, IL (US)

(21) Appl. No.: 14/422,756

(22) PCT Filed: Aug. 20, 2013
(86) PCT No.: PCT/US13/55854

§ 371 Date: Feb. 20, 2015

Related U.S. Application Data
(60) Provisional application No. 61/691,159, filed on Aug. 20, 2012.
(60) Provisional application No. 61/752,171, filed on Jan. 14, 2013.

(51) Int. Cl. A61K 31/4245; A61K 45/06

(52) U.S. Cl. CPC A61K 31/4245; A61K 45/06

(57) ABSTRACT

Methods and compositions are provided for treating or preventing a Gram-positive bacteria infection using an inhibitor of lipo-teichoic acid synthase (LtaS). In some embodiments, the inhibitor is a small molecule. In certain embodiments, the inhibitor is 2-oxo-2-(5-phenyl-1,3,4-oxadiazol-2-ylamino)ethyl 2-naphtho[2,1-b]furan-1 -ylacetate, or a salt thereof.

### CROSS-REFERENCE TO RELATED APPLICATIONS

This Application claims priority to U.S. Provisional Patent Application No. 61/691159 filed on Aug. 20, 2012 and U.S. Provisional Patent Application No. 61/752171 filed on Jan. 14, 2013, both of which are hereby incorporated by reference in their entirety.

### STATEMENT REGARDING FEDERALLY SPONSORED RESEARCH OR DEVELOPMENT

This invention was made with government support under grant U54-AI-057153 awarded by the NIAID. The government has certain rights in the invention.

### TECHNICAL FIELD

Embodiments of this invention are directed generally to microbiology and medicine. In certain aspects there are methods and compositions relating to treatment of Staphylococcus infection.

### BACKGROUND

Staphylococcus aureus is a commensal of the human skin and nares as well as an invasive pathogen causing soft tissue infections, sepsis, endocarditis and pneumonia (Lowy 1998). Owing to the frequent use of antibiotics, staphylococci frequently evolved resistance to drugs (DeLeo et al. 2010 and Neu 1992). These methicillin-resistant S. aureus strains (MRSA) are associated with therapeutic failure and increased mortality of staphylococcal infections (Klevens et al. 2007 and Klevens et al. 2008). Of note, vancomycin-resistance recently emerged due to the transfer of resistance genes from glycopeptide (vancomycin)-resistant enterococci to MRSA strains (Weigel et al. 2003). The resulting VRSA strains are virtually resistant to all available antibiotics (Tenover et al. 2001). Expanded use of antibiotics in Asia has triggered increases in community-acquired infections with MRSA and the emergence hypervirulent strains, indicating that drug-resistant Gram-positive bacteria represent a global threat (Li et al. 2012). Two drugs, daptomycin and linezolid, have been recently been licensed for the treatment of MRSA infections (Arbeit et al. 2004 and Stevens et al. 2002). However, MRSA strains already evolved resistance against these new antibiotics, revealing the continuous need for new drug targets and for the development of new antibiotics to combat S. aureus infections (van Hal and Paterson 2011).

The crisis in antibiotic resistance applies not only to MRSA but also to other Gram-positive bacteria causing significant clinical disease, for example vancomycin (glycopeptide)-resistant enterococci (E. faecium and E. faecalis), as well as drug-resistant Staphylococcus epidermidis, Clostridum difficile, and Streptococcus pneumoniae (Willems et al. 2011).

Therefore, there is an increased need for developing alternative antibiotics that are structurally unrelated to known antibiotics and that are targeting novel pathways in pathogens.

### BRIEF SUMMARY OF THE INVENTION

Staphylococcus aureus remains a leading cause of infectious disease morbidity and mortality. Lipoteichoic acid (LTA) is an abundant secondary cell wall polymer of Gram-positive bacteria and consists of repeating units of 1,3-glycerol phosphate or polyglycerophosphate, linked to a membrane anchor. LTA synthase (LtaS) catalyzes the transfer of glycerophosphate from phosphatidylglycerol to the growing chain of polyglycerophosphate on the trans side of the plasma membrane. Genetic disruption of polyglycerophosphate synthesis is poorly tolerated and loss of LTA leads to cessation of bacterial growth.

Methods and compositions concern an LtaS inhibitor for preventing or treating a Gram-positive infection. In some embodiments, the LtaS inhibitor has the following structure:

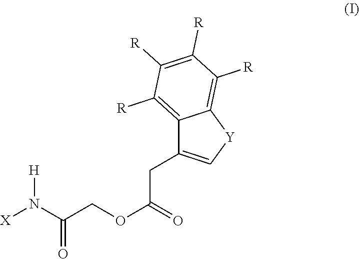

wherein X is an aryl, heteroaryl substituted aryl, or substituted heteroaryl group;
Y is O or NH;
and each R is independently H, alkyl, aryl, or heteroaryl or two adjacent R groups form a saturated or unsaturated carbocyclic or heterocyclic ring, and wherein at least one R is not H. In particular embodiments, X is a heteroaryl group, which may or may not be substituted; or a salt thereof. In certain embodiments, a heteroaryl group is substituted with a phenyl or other ring structure group. In further embodiments, the LtaS inhibitor is 2-oxo-2-(5-phenyl-1,3,4-oxadiazol-2-ylamino)ethyl 2-naphtho[2,1-b]furan-1-ylacetate, or a salt thereof.

In some embodiments, an LtaS inhibitor comprises a compound of formula I or a pharmaceutically acceptable salt thereof. In specific embodiments, the hydrophobic double-ring structure is naphthofuran. In certain embodiments, the compound is 2-oxo-2-(5-phenyl-1,3,4-oxadiazol-2-ylamino)ethyl 2-naphtho[2,1-b]furan-1-ylacetate, or a salt thereof.

Certain embodiments are directed to methods of inhibiting a Gram-positive bacterial infection in a patient comprising administering to the patient a composition comprising a lipoteichoic acid synthesis inhibitor, for example, a lipoteichoic acid synthase (LtaS) inhibitor, that comprises an oxadiazole ring. In particular embodiments, the patient is administered an amount of the composition that has been previously shown to be an effective amount.

Additional embodiments concern methods for inhibiting lipoteichoic acid synthesis in a Gram-positive bacteria comprising administering to the bacteria a lipoteichoic acid synthesis inhibitor, for example, a lipoteichoic acid synthase (LtaS) inhibitor, that comprises an oxadiazole ring.

In other embodiments there are methods for inhibiting Gram-positive bacteria comprising administering to the bacteria a compound having the following structure:

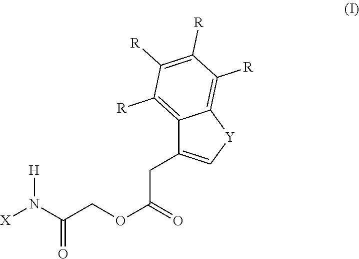

wherein X is an aryl group;
Y is O or NH;
and each R is independently H, alkyl, aryl, or heteroaryl or two adjacent R groups form a saturated or unsaturated carbocyclic or heterocyclic ring, and wherein at least one R is not H. In particular embodiments, X is an oxadiazole. In specific embodiments, the compound is 2-oxo-2-(5-phenyl-1,3,4-oxadiazol-2-ylamino)ethyl 2-naphtho[2,1-b]furan-1-ylacetate, or a salt thereof. In some embodiments, the inhibitor is a compound of formula I, or a salt thereof. In some embodiments, methods for inhibiting lipoteichoic acid synthesis in a Gram-positive bacteria comprising administering to the bacteria a compound of formula I are provided.

In some embodiments, a method for inhibiting lipoteichoic acid synthesis in a Gram-positive bacteria comprises administering a compound having the formula:

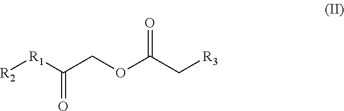

wherein R₁ is methylene or NH; R₂ is a hydrogen atom, a benzyl group or an oxadiazole ring; and R₃ is a benzofuran, a naphthofuran, an indole group, or a phenyl ring. In some embodiments, methods and compositions comprise administration of an LtaS inhibitor of formula II. Certain embodiments are directed to methods of inhibiting a Gram-positive bacterial infection in a patient comprising administering to the patient a composition comprising a compound of formula II, or a pharmaceutically acceptable salt thereof.

In some embodiments, R₂ is a 2-substituted oxadiazole ring. In other embodiments, the oxadiazole ring is substituted with a phenyl ring. In a further embodiment, the benzyl group is a substituted benzyl group. In yet other embodiments, the benzyl group is substituted with an alkyl group at the benzylic position. In some embodiments, the benzofuran is substituted at adjacent positions with two alkyl groups. In some embodiments, the two alkyl groups together form a ring. In a further embodiment, R₁ is methylene, R₂ is a hydrogen atom, and R₃ is a naphthofuran. In yet a further embodiment, R1 is NH, R₂ is a benzyl group substituted at the benzylic position with an alkyl group, and R₃ is a naphthofuran. In some embodiments R1 is NH, R₂ is an oxadiazole ring substituted with a phenyl ring, and R3 is a phenyl ring, a naphthofuran, an indole group, or a benzofuran substituted at adjacent positions with two alkyl groups, wherein the two alkyl groups may together form a ring.

In specific embodiments, the compound is 2-oxo-2-(5-phenyl-1,3,4-oxadiazol-2-ylamino)ethyl 2-(naphtho[2,1-b]furan-1-yl)acetate. In specific embodiments, the compound is 2-oxopropyl 2-(naphtho[2,1-b]furan-1-yl)acetate. In specific embodiments, the compound is 2-(methylamino)-2-oxoethyl 2-(naphtho[2,1-b]furan-1-yl)acetate. In specific embodiments, the compound is 2-oxo-2-(1-phenylethylamino)ethyl 2-(naphtho[2,1-b]furan-1-yl)acetate. In specific embodiments, the compound is 2-oxo-2-(5-phenyl-1,3,4-oxadiazol-2-ylamino)ethyl 2-phenylacetate. In specific embodiments, the compound is 2-oxo-2-(5-phenyl-1,3,4-oxadiazol-2-ylamino)ethyl 2-(1H-indol-3-yl)acetate. In specific embodiments, the compound is 2-oxo-2-(5-phenyl-1,3,4-oxadiazol-2-ylamino)ethyl 2-(5,6-dimethylbenzofuran-3-yl)acetate. In specific embodiments, the compound is 2-oxo-2-(5-phenyl-1,3,4-oxadiazol-2-ylamino)ethyl 2-(6,7-dihydro-5H-indeno[5,6-b]furan-3-yl)acetate. In some embodiments, methods for inhibiting lipoteichoic acid synthesis in a Gram-positive bacteria comprising administering to the bacteria a lipoteichoic acid synthesis inhibitor, for example, a lipoteichoic acid synthase (LtaS) inhibitor, that comprises an oxadiazole ring are provided. In some embodiments, methods for inhibiting lipoteichoic acid synthesis in a Gram-positive bacteria comprising administering to the bacteria a compound of formula II are provided.

In some embodiments, a method of inhibiting a Gram-positive bacterial infection in a patient comprising administering to the patient a composition comprising a lipoteichoic acid synthesis inhibitor, for example, a lipoteichoic acid synthase (LtaS) inhibitor, that comprises a compound having the formula:

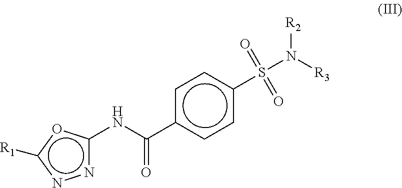

wherein R1 is an alkyl group, cycloalkyl group, alkyl ether, alkyl thioether, phenyl ring, or heterocycle; and R2 and R3 are each, independently, an alkyl group, a benzyl group, a phenyl group, or R2 and R3 together form part of a piperidine ring. In some embodiments, methods and compositions comprise administration of an LtaS inhibitor of formula III. Certain embodiments are directed to methods of inhibiting a Gram-positive bacterial infection in a patient comprising administering to the patient a composition comprising a compound of formula III, or a pharmaceutically acceptable salt thereof.

In some embodiments, R1 is an alkyl group, cycloalkyl group, alkyl ether, alkyl thioether, phenyl group, or heterocycle. In a further embodiment, the phenyl ring is substituted with one or more alkoxy groups. In some embodiments, R2 and R3 are each, independently an alkyl group, a benzyl group, or a phenyl group. In further embodiments, R2 and R3 together form part of a piperidine ring. In some embodiments, the piperidine ring is substituted with an alkyl group.

In specific embodiments, the compound is 4-(2-methylpiperidin-1-ylsulfonyl)-N-(5-(methylthiomethyl)-1,3,4-oxadiazol-2-yl)benzamide. In specific embodiments, the compound is 4-(N-benzyl-N-isopropylsulfamoyl)-N-(5-(3-methoxyphenyl)-1,3,4-oxadiazol-2-yl)benzamide. In specific embodiments, the compound is 4-(2-methylpiperidin-1-ylsulfonyl)-N-(5-(thiophen-2-yl)-1,3,4-oxadiazol-2-yl)benzamide. In specific embodiments, the compound is 4-(N-butyl-N-ethylsulfamoyl)-N-(5-(methoxymethyl)-1,3,4-oxadiazol-2-yl)benzamide. In specific embodiments, the compound is 4-(N,N-diisobutylsulfamoyl)-N-(5-methyl-1,3,4-oxadiazol-2-yl)benzamide. In specific embodiments, the compound is N-(5-(3,5-dimethoxyphenyl)-1,3,4-oxadiazol-2-yl)-4-(N-methyl-N-phenylsulfamoyl)benzamide. In specific embodiments, the compound is N-(5-cyclopropyl-1,3,4-oxadiazol-2-yl)-4-(2-methylpiperidin-1-ylsulfonyl)benzamide.

Additional embodiments concern methods for inhibiting lipoteichoic acid synthesis in a Gram positive bacteria comprising administering to the bacteria a lipoteichoic acid synthesis inhibitor, for example, a lipoteichoic acid synthase (LtaS) inhibitor, that comprises an oxadiazole ring.

In further embodiments there are methods of treating a subject having or at risk of developing a Gram-positive bacteria infection comprising administering an effective amount of a pharmaceutically acceptable composition comprising an LtaS inhibitor having an oxadiazole ring. Further embodiments concern methods for inhibiting lipoteichoic acid synthesis in a Gram-positive bacteria comprising administering to the bacteria a lipoteichoic acid synthesis inhibitor, for example, a lipoteichoic acid synthase (LtaS) inhibitor, that comprises a 2-oxadiazolyl-4-(sulfonamide)benzamide. In some embodiments, methods for inhibiting lipoteichoic acid synthesis in a Gram positive bacteria comprising administering to the bacteria a compound of formula III are provided.

In some embodiments, there is a method of inhibiting a Gram-positive bacteria infection in a patient comprising administering to the patient a composition comprising a lipoteichoic acid synthesis inhibitor, for example, a lipoteichoic acid synthase (LtaS) inhibitor, that comprises a compound having the formula:

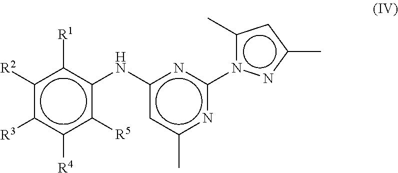

wherein R₁, R₂, R₃, R₄, and R₅ are each, independently, a hydrogen atom, an alkyl group or a halogen atom, provided at least 1 position on the phenyl ring is substituted with an alkyl group or halogen atom. In some embodiments, at least one of R₂, R₃ or R₅ are substituted with an alkyl group or a halogen atom. In some embodiments, methods and compositions comprise administration of an LtaS inhibitor of formula IV. Certain embodiments are directed to methods of inhibiting a Gram-positive bacterial infection in a patient comprising administering to the patient a composition comprising a compound of formula IV, or a pharmaceutically acceptable salt thereof.

In specific embodiments, the compound is 2-(3,5-dimethyl-1H-pyrazol-1-yl)-6-methyl-N-o-tolylpyrimidin-4-amine. In specific embodiments, the compound is 2-(3,5-dimethyl-1H-pyrazol-1-yl)-N-(5-fluoro-2-methylphenyl)-6-methylpyrimidin-4-amine. In specific embodiments, the compound is 2-(3,5-dimethyl-1H-pyrazol-1-yl)-6-methyl-N-m-tolylpyrimidin-4-amine. In specific embodiments, the compound is 2-(3,5-dimethyl-1H-pyrazol-1-yl)-6-methyl-N-p-tolylpyrimidin-4-amine. In some embodiments, methods for inhibiting lipoteichoic acid synthesis in a Gram-positive bacteria comprising administering to the bacteria a lipoteichoic acid synthesis inhibitor, for example, a lipoteichoic acid synthase (LtaS) inhibitor, that comprises a 4-methylpyrimidine ring are provided. In some embodiments, methods for inhibiting lipoteichoic acid synthesis in a Gram-positive bacteria comprising administering to the bacteria a compound of formula IV are provided.

In some embodiments, there is a method of inhibiting a Gram-positive bacteria infection in a patient comprising administering to the patient a composition comprising a lipoteichoic acid synthesis inhibitor, for example, a lipoteichoic acid synthase (LtaS) inhibitor, that comprises a compound having the formula:

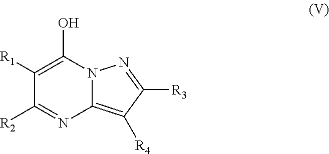

wherein R₁ is a hydrogen atom or a substituted or unsubstituted benzyl group, R₂ is an alkyl group or a substituted or unsubstituted phenyl group, R₃ is a hydrogen atom or an alkyl group, and R₄ is a phenyl group, an N-benzyl amide, or an N-2-(phenyl)-ethyl group. In some embodiments, methods and compositions comprise administration of an LtaS inhibitor of formula V. Certain embodiments are directed to methods of inhibiting a Gram-positive bacterial infection in a patient comprising administering to the patient a composition comprising a compound of formula V, or a pharmaceutically acceptable salt thereof.

In some embodiments, R₁ is 4-chlorobenzyl, 3-chlorobenzyl, or 2-chloro-4-fluorobenzyl. In some embodiments, R₂ is a methyl group or 4-chlorophenyl. In some embodiments, R₃ is a hydrogen atom or methyl. In some embodiments, R₄ is phenyl, N-2-chlorobenzyl amide, N-4-methylbenzyl amide, N-(phenylethyl) amide, or N-(4-chlorophenethyl) amide.

In some embodiments, the compound is 6-(4-chlorobenzyl)-N-(4-chlorophenethyl)-7-hydroxy-5-methylpyrazolo[1,5-a]pyrimidine-3-carboxamide. In some embodiments, the compound is N-(2-chlorobenzyl)-6-(3-chlorobenzyl)-7-hydroxy-5-methylpyrazolo[1,5-a]pyrimidine-3-carboxamide. In some embodiments, the compound is 6-(2-chloro-4-fluorobenzyl)-7-hydroxy-5-methyl-N-(4-methylbenzyl)pyrazolo[1,5-a]pyrimidine-3-carboxamide. In some embodiments, the compound is 6-(2-chloro-4-fluorobenzyl)-7-hydroxy-5-methyl-N-(4-methylbenzyl)pyrazolo[1,5-a]pyrimidine-3-carboxamide. In some embodiments, the compound is 6-(2-chloro-4-fluorobenzyl)-7-hydroxy-5-methyl-N-phenethylpyrazolo[1,5-a]pyrimidine-3-carboxamide. In some embodiments, methods for inhibiting lipoteichoic acid synthesis in a Gram-positive bacteria comprising administering to the bacteria a lipoteichoic acid synthesis inhibitor, for example, a lipoteichoic acid synthase (LtaS) inhibitor, that comprises a pyrazolo[1,5-a]pyrimidine bicyclic are provided. In some embodiments, methods for inhibiting lipoteichoic acid synthesis in a Gram-positive bacteria comprising administering to the bacteria a compound of formula V are provided.

In some embodiments, there is a method of inhibiting a Gram-positive bacteria infection in a patient comprising administering to the patient a composition comprising a lipoteichoic acid synthesis inhibitor, for example, a lipoteichoic acid synthase (LtaS) inhibitor, that comprises a compound having the formula:

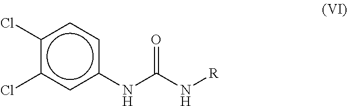

wherein R is 1-ethyl-3-iminoindolin-2-one or 5-isopropyloxazol-4(5H)-one. In some embodiments, the compound is (Z)—N-(3,4-dichlorophenyl)-2-(1-ethyl-2-oxoindolin-3-ylidene)hydrazinecarboxamide. In some embodiments, the compound is 1-(3,4-dichlorophenyl)-3-(5-isopropyl-4-oxo-4,5-dihydrooxazol-2-yl)urea. In some embodiments, methods and compositions comprise administration of an LtaS inhibitor of formula VI. Certain embodiments are directed to methods of inhibiting a Gram-positive bacterial infection in a patient comprising administering to the patient a composition comprising a compound of formula VI, or a pharmaceutically acceptable salt thereof.

In some embodiments, the compound is (Z)—N-(3,4-dichlorophenyl)-2-(1-ethyl-2-oxoindolin-3-ylidene)hydrazinecarboxamide. in some embodiments, the compound is 1-(3,4-dichlorophenyl)-3-(5-isopropyl-4-oxo-4,5-dihydrooxazol-2-yl)urea. In some embodiments, methods for inhibiting lipoteichoic acid synthesis in a Gram-positive bacteria comprising administering to the bacteria a lipoteichoic acid synthesis inhibitor, for example, a lipoteichoic acid synthase (LtaS) inhibitor, that comprises a 1-(3,4-dichlorophenyl)urea are provided. In some embodiments, methods for inhibiting lipoteichoic acid synthesis in a Gram-positive bacteria comprising administering to the bacteria a compound of formula VI are provided.

In some embodiments, there is a method of inhibiting a Gram-positive bacteria infection in a patient comprising administering to the patient a composition comprising a lipoteichoic acid synthesis inhibitor, for example, a lipoteichoic acid synthase (LtaS) inhibitor, that comprises a compound having the formula:

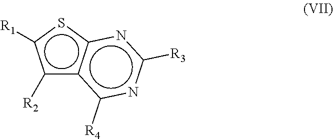

wherein R₁ and R₂ are each, independently, an alkyl group or R₁ and R₂ together form a carbocyclic ring, R₃ is a pyridinyl group or an alkylamine, and R₄ is a thiol group or a N-methyl-1-(5-methylfuran-2-yl)methanamine group. In some embodiments, methods and compositions comprise administration of an LtaS inhibitor of formula VII. Certain embodiments are directed to methods of inhibiting a Gram-positive bacterial infection in a patient comprising administering to the patient a composition comprising a compound of formula VII, or a pharmaceutically acceptable salt thereof.

In some embodiments, the compound is 5,6-(cycloehexyl)-4-[N-methyl-1-(5-methylfuran-2-yl)methanamine]-2-(3-pyridinyl)-thieno[2,3-d]pyrimidine. In some embodiments, the compound is 5,6-dimethyl-2-(N,N-diethyl)methaneamine-thieno[2,3-d]pyrimidine-4thiol. In some embodiments, methods for inhibiting lipoteichoic acid synthesis in a Gram-positive bacteria comprising administering to the bacteria a lipoteichoic acid synthesis inhibitor, for example, a lipoteichoic acid synthase (LtaS) inhibitor, that comprises a thieno[2,3-d]pyrimidine are provided. In some embodiments, methods for inhibiting lipoteichoic acid synthesis in a Gram-positive bacteria comprising administering to the bacteria a compound of formula VII are provided.

In some embodiments, there is a method of inhibiting a Gram-positive bacteria infection in a patient comprising administering to the patient a composition comprising a lipoteichoic acid synthesis inhibitor, for example, a lipoteichoic acid synthase (LtaS) inhibitor, that comprises a compound having the formula:

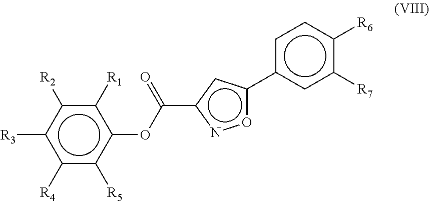

wherein R₁, R₂, R₃, R₄, and R₅ are each, independently, a hydrogen atom, an alkyl group or a halogen atom, provided at least 1 position on the phenyl ring is substituted with an alkyl group or halogen atom; and R₆ and R₇ are each, independently, an alkyl group or R₆ and R₇ together form a carbocylic ring, wherein one or more carbon atoms can be replaced with two heteroatoms, provided that two oxygens are not adjacent to each other. In some embodiments, at least one of R₁, R₂ or R₄ are substituted with an alkyl group or a halogen atom. In some embodiments R₆ and R₇ are alkyl groups. In some embodiments, R₆ and R₇ form a 6 membered carbocyclic ring wherein oxygen atoms are present at the R₆ and R₇ positions. in some embodiments, at least one of R₁, R₂ or R₄ are substituted with an alkyl group or a halogen atom and R₆ and R₇ are alkyl groups. In some embodiments at least one of R₁, R₂ or R₄ are substituted with an alkyl group or a halogen atom and R₆ and R₇ form a 6 membered carbocyclic ring wherein oxygen atoms are present at the R₆ and R₇ positions. In some embodiments, methods and compositions comprise administration of an LtaS inhibitor of formula VIII. Certain embodiments are directed to methods of inhibiting a Gram-positive bacterial infection in a patient comprising administering to the patient a composition comprising a compound of formula VIII, or a pharmaceutically acceptable salt thereof.

In some embodiments, the compound is 2-chlorophenyl 5-(3,4-dimethylphenyl)isoxazole-3-carboxylate. In some embodiments, the compound is m-tolyl 5-(3,4-dimethylphenyl)isoxazole-3-carboxylate. In some embodiments, the compound is m-tolyl 5-(2,3-dihydrobenzo[b][1,4]dioxin-6-yl)isoxazole-3-carboxylate. In some embodiments, methods for inhibiting lipoteichoic acid synthesis in a Gram-positive bacteria comprising administering to the bacteria a lipoteichoic acid synthesis inhibitor, for example, a lipoteichoic acid synthase (LtaS) inhibitor, that comprises an isoxazole are provided. In some embodiments, methods for inhibiting lipoteichoic acid synthesis in a Gram-positive bacteria comprising administering to the bacteria a compound of formula VIII are provided.

In some embodiments, there is a method of inhibiting a Gram-positive bacteria infection in a patient comprising administering to the patient a composition comprising a lipoteichoic acid synthesis inhibitor, for example, a lipoteichoic acid synthase (LtaS) inhibitor, that comprises a compound having the formula:

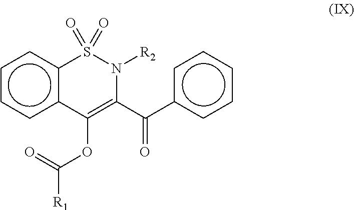

wherein R1 is alkyl or furanyl and R2 is alkyl. In some embodiments, R₁ is methyl, ethyl or 2-furanyl. In some embodiments, R2 is ethyl or butyl. In some embodiments, methods and compositions comprise administration of an LtaS inhibitor of formula IX. Certain embodiments are directed to methods of inhibiting a Gram-positive bacterial infection in a patient comprising administering to the patient a composition comprising a compound of formula IX, or a pharmaceutically acceptable salt thereof.

In some embodiments, the compound is (3-benzoyl-2-butyl-1,1-dioxo-1-lambda⁶,2-benzothiazin-4-yl) propanoate. In some embodiments, the compound is (3-benzoyl-2-butyl-1,1-dioxo-1-lambda⁶,2-benzothiazin-4-yl) acetate. In some embodiments, the compound is (3-benzoyl-2-ethyl-1,1-dioxo-1-lambda⁶,2-benzothiazin-4-yl)2-furanoate. In some embodiments, methods for inhibiting lipoteichoic acid synthesis in a Gram-positive bacteria comprising administering to the bacteria a lipoteichoic acid synthesis inhibitor, for example, a lipoteichoic acid synthase (LtaS) inhibitor, that comprises a dioxo-1-lambda⁶,2-benzothiazinyl are provided. In some embodiments, methods for inhibiting lipoteichoic acid synthesis in a Gram-positive bacteria comprising administering to the bacteria a compound of formula IX are provided.

In some embodiments, there is a method of inhibiting a Gram-positive bacteria infection in a patient comprising administering to the patient a composition comprising a lipoteichoic acid synthesis inhibitor, for example, a lipoteichoic acid synthase (LtaS) inhibitor, that comprises a compound having the formula:

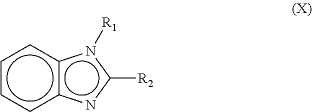

wherein R₁ is alkyl and R₂ is N-cyclopentanecarboxamide or 1-(naphthalen-1-yl)pyrrolidin-2-one. In some embodiments, methods and compositions comprise administration of an LtaS inhibitor of formula X. Certain embodiments are directed to methods of inhibiting a Gram-positive bacterial infection in a patient comprising administering to the patient a composition comprising a compound of formula X, or a pharmaceutically acceptable salt thereof.

In some embodiments, the compound is 4-(1-methyl-1H-benzo[d]imidazol-2-yl)-1-(naphthalen-1-yl)pyrrolidin-2-one. In some embodiments, the compound is N-(1-propyl-1H-benzo[d]imidazol-2-yl)cyclopentanecarboxamide. In some embodiments, methods for inhibiting lipoteichoic acid synthesis in a Gram-positive bacteria comprising administering to the bacteria a lipoteichoic acid synthesis inhibitor, for example, a lipoteichoic acid synthase (LtaS) inhibitor, that comprises a 1H-benzo[d]imidazole are provided. In some embodiments, methods for inhibiting lipoteichoic acid synthesis in a Gram-positive bacteria comprising administering to the bacteria a compound of formula X are provided.

In some embodiments, there is a method of inhibiting a Gram-positive bacteria infection in a patient comprising administering to the patient a composition comprising a lipoteichoic acid synthesis inhibitor, for example, a lipoteichoic acid synthase (LtaS) inhibitor, that comprises a compound having the formula:

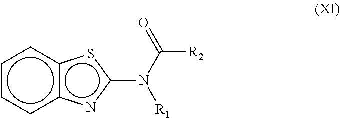

wherein R₁ is H and R₂ is 1,1,1,3,3,3-hexafluoro-2-methoxypropan-2-ylamine or R₁ and R₂ may join to form 4-isopentyl-3-methyl-1H-pyrazol-5(4H)-one. In some embodiments, methods and compositions comprise administration of an LtaS inhibitor of formula XI. Certain embodiments are directed to methods of inhibiting a Gram-positive bacterial infection in a patient comprising administering to the patient a composition comprising a compound of formula XI, or a pharmaceutically acceptable salt thereof.

In some embodiments, the compound is 1-(benzo[d]thiazol-2-yl)-3-(1,1,1,3,3,3-hexafluoro-2-methoxypropan-2-yl)urea. In some embodiments, the compound is 1-(benzo[d]thiazol-2-yl)-4-isopentyl-3-methyl-1H-pyrazol-5(4H)-one. In some embodiments, methods for inhibiting lipoteichoic acid synthesis in a Gram-positive bacteria comprising administering to the bacteria a lipoteichoic acid synthesis inhibitor, for example, a lipoteichoic acid synthase (LtaS) inhibitor, that comprises a benzo[d]thiazole are provided. In some embodiments, methods for inhibiting lipoteichoic acid synthesis in a Gram-positive bacteria comprising administering to the bacteria a compound of formula XI are provided.

In some embodiments, there is a method of inhibiting a Gram-positive bacteria infection in a patient comprising administering to the patient a composition comprising a lipoteichoic acid synthesis inhibitor, for example, a lipoteichoic acid synthase (LtaS) inhibitor, that comprises a compound having the formula:

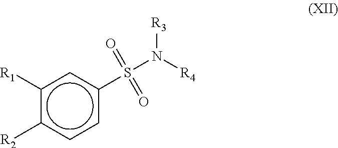

wherein R₁ is a hydrogen atom or an N-alkyl-N-amide and R₂ is an akyl or an N-alkyl-N-amide, R₃ is alkyl or 5-chloro-1-ethyl-3-iminoindolin-2-one and R₄ is hydrogen or benzyl. In some embodiments, R₁ and R₂ join to form a bis-lactam ring. In some embodiments, methods and compositions comprise administration of an LtaS inhibitor of formula XII. Certain embodiments are directed to methods of inhibiting a Gram-positive bacterial infection in a patient comprising administering to the patient a composition comprising a compound of formula XII, or a pharmaceutically acceptable salt thereof.

In some embodiments, the compound is N-benzyl-N,1,5-trimethyl-2,4-dioxo-2,3,4,5-tetrahydro-1H-benzo[b][1,4]diazepine-7-sulfonamide. In some embodiments, the compound is (E)-N′-(5-chloro-1-ethyl-2-oxoindolin-3-ylidene)-4-methylbenzenesulfonohydrazide. In some embodiments, methods for inhibiting lipoteichoic acid synthesis in a Gram-positive bacteria comprising administering to the bacteria a lipoteichoic acid synthesis inhibitor, for example, a lipoteichoic acid synthase (LtaS) inhibitor, that comprises a benzenesulfonamide are provided. In some embodiments, methods for inhibiting lipoteichoic acid synthesis in a Gram-positive bacteria comprising administering to the bacteria a compound of formula XII are provided.

In some embodiments methods and compositions concern an LtaS inhibitor that comprises a compound of any of formula I through XII, or a pharmaceutically acceptable salt thereof, for preventing or treating a Gram-positive infection. Additional embodiments concern methods for inhibiting lipoteichoic acid synthesis in a Gram-positive bacteria comprising administering to the bacteria a lipoteichoic acid synthesis inhibitor, for example, a lipoteichoic acid synthesis inhibitor, for example, a lipoteichoic acid synthase (LtaS) inhibitor, that comprises a compound of any of formula I through XII, or a pharmaceutically acceptable salt thereof. Methods and compositions concern an LtaS inhibitor that comprises a compound of any of formula I through XII, or a pharmaceutically acceptable salt thereof, for preventing or treating a Gram-positive infection. Additional embodiments concern methods for inhibiting lipoteichoic acid synthesis in a Gram-positive bacteria comprising administering to the bacteria a lipoteichoic acid synthesis inhibitor, for example, a lipoteichoic acid synthase (LtaS) inhibitor, that comprises a compound of any of formula I through XII, or a pharmaceutically acceptable salt thereof. In other embodiments there are methods for inhibiting Gram-positive bacteria comprising administering to the bacteria a compound of any of formula I through XII, or a pharmaceutically acceptable salt thereof.

In some embodiments, methods relate to an infection in which the Gram-positive bacteria is Staphylococcus aureus. In further embodiments, the Staphylococcus aureus is a drug resistant Staphylococcus aureus. In certain embodiments, the drug resistant Staphylococcus aureus is methicillin-resistant Staphylococcus aureus (MRSA). In other embodiments, methods relate to an infection in which the Gram-positive bacteria is Bacillus anthracis. In additional embodiments, the Gram-positive bacteria is Enterococcus faecium. In further embodiments, the Gram-positive bacteria is vancomycin-resistant Enterococcus faecium.

In other embodiments, methods may apply to other Gram-positive bacteria whose group includes, but is not limited to, Staphylococcus epidermidis, Staphylococcus saprophyticus, Streptococcus pneumonia, Streptococcus pyrogens, Streptococcus agalactiae, Enterococcim Streptococcus viridians, Clostridium tetani, Clostridium botulinum, Clostridium perfringes, Clostridium difficile, Clostridiun scindens, Bacillus anthracis, Bacillus cereus, and Listeria monocytogenes. In certain embodiments, an LtaS inhibitor is more effective at inhibiting certain Gram-positive bacteria, such as Staphylococcus aureus, Bacillus anthracis and/or Enterococcus faecium, relative to Gram-negative bacteria, such as E. coli or any other Gram-negative bacteria (particularly those discussed in this paragraph). In some embodiments, the LtaS inhibitor has less than about 50, 40, 30, 20, 10, 5 or less percent of the inhibiting activity against E. coli as compared to Staphylococcus aureus, Bacillus anthracis and/or Enterococcus faecium (or any other Gram-positive bacteria) (based on assays described, for instance, in the Examples).

In some embodiments, methods concern an LtaS inhibitor that comprises a compound of any of formula I through XII, or a pharmaceutically acceptable salt thereof, that disturbs cell envelope architecture. In other embodiments, the LtaS inhibitor inhibits heterologous LTA synthesis in E. coli. In additional embodiments, the LtaS inhibitor competes with phosphatidylglycerol (PG).

In additional embodiments, there are methods that further comprise administering a second anti-microbial treatment. Some aspects concern a second anti-microbial treatment that is an antibiotic agent, an anti-infective agent, a passive vaccine or an active vaccine.

In additional methods, the patient has been determined to have a Gram-positive bacterial infection. Methods may involve identifying the patient as having a Gram-positive bacterial infection. They may also involve selecting the patient after the patient is diagnosed with a Staphylococcal infection. In some embodiments, there are methods in which the patient has been determined to have or be at risk of developing a Gram-positive bacterial infection. Some embodiments include testing the patient for a Gram-positive bacterial infection. For instance, some aspects further concern obtaining from the patient a biological sample for testing whether the patient has a Gram-positive bacterial infection.

In some methods the patient is at risk of a Gram-positive bacterial infection, particularly a Gram-positive bacteria that can lead to a medical issue, condition or disease, such as one requiring treatment. In particular examples the patient is at risk for a Staphylococcus bacterial infection. Some aspects concern a patient who is immune deficient, is immune-compromised, is hospitalized, is undergoing an invasive medical procedure, is infected with influenza virus or is on a respirator. In particular situations, the patient has pneumonia, sepsis, corneal infection, skin infection, infection of the central nervous system, or toxic shock syndrome. In certain instances, the patient exhibits a skin abscess, a boil, or a furuncle.

In some embodiments, there are methods that involve monitoring the patient for the Gram-positive bacterial infection within a week of first administering a therapeutic composition or LtaS inhibitor.

The methods can include treating a subject having or at risk of developing a Staphylococcal infection comprising administering an effective amount of an LtaS inhibitor to a subject having or at risk of developing a Staphylococcal infection.

The methods can also include inhibiting, attenuating, treating, or ameliorating toxic-shock syndrome and its related pathology.

In a further aspect, an LtaS inhibitor that comprises a compound of any of formula I through XII, or a pharmaceutically acceptable salt thereof, is administered orally, topically, nasally, intravenously, intravascularly, intrathecally, intratracheally, by inhalation, or by instillation. The LtaS inhibitor can be administered to various organs or tissues including, but not limited to the subject's skin, respiratory tract (including the lungs) kidneys, central nervous system, reproductive organs, vagina, or eyes.

In certain aspects, the Staphylococcal infection is a Staphylococcus aureus infection. In a further aspect the Staphylococcus aureus infection is a drug resistant Staphylococcus aureus infection. In still another aspect the drug resistant Staphylococcus aureus infection is a methicillin-resistant Staphylococcus aureus (MRSA) infection.

The method can also further comprise the step of testing the patient for a pathogenic bacterial infection. The method can include obtaining from the patient a biological sample for testing whether the patient has a pathogenic bacterial infection. In additional embodiments, the patient is tested for the type of pathogenic bacterial infection. In certain embodiments, the patient is tested for MRSA or pneumonia.

In certain aspects of the methods the patient is determined to have a Staphylococcal infection. The methods can further comprise identifying the patient as having a Staphylococcal infection. In a further aspect the method can further comprise selecting the patient after the patient is diagnosed with a Staphylococcal infection. The method can also further comprise the step of testing the patient for a Staphylococcal infection. The method can include obtaining from the patient a biological sample for testing whether the patient has a Staphylococcal infection. In additional embodiments, the patient is tested for the type of Staphylococcal infection. In certain embodiments, the patient is tested for MRSA or pneumonia.

In certain aspects of the methods the patient is determined to have a Streptococcus or Staphylococcus infection or some other Gram-positive bacterial infection. The methods can further comprise identifying the patient as having a Staphylococcus or Streptococcus infection. In a further aspect the method can further comprise selecting the patient after the patient is diagnosed with a Streptococcus infection. The method can also further comprise the step of testing the patient for a Streptococcus infection. The method can include obtaining from the patient a biological sample for testing whether the patient has a Streptococcus infection. In additional embodiments, the patient is tested for the type of Streptococcus infection. In certain embodiments, the patient is tested for pneumonia.

A patient is a human patient in some embodiments. It is contemplated that any embodiment involving a patient may also be applied to a subject, which refers to any organism that suffers physiologically as a result from infection by a Gram-positive bacteria.

In certain embodiments, the subject is a mammal, which includes but is not limited to dogs, cats, cows, horses, pigs, monkeys, and sheep.

In certain aspects a patient is administered an LtaS inhibitor within at least about, at most about, or about 1, 2, 3, 4, 5, 6, 7, 8, 9, or 10 hours, days, or weeks of being determined to have a Gram-positive infection.

Methods may involve administering a composition containing about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 nanograms (ng), micrograms (mcg), milligrams (mg), or grams of an LtaS inhibitor, or any range derivable therein.

Alternatively, embodiments may involve providing or administering to the patient or to cells or tissue of the patient about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 nanograms (ng), micrograms (mcg), milligrams (mg), or grams of an LtaS inhibitor that comprises a compound of any of formula I through XII, or a pharmaceutically acceptable salt thereof, or any range derivable therein, in one dose or collectively in multiple doses. In some embodiments, the composition comprises between about 0.1 ng and about 2.0 g of an LtaS inhibitor.

Alternatively, the composition may have a concentration of LtaS inhibitor that comprises a compound of any of formula I through XII, or a pharmaceutically acceptable salt thereof, that is 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000 μg/ml or mg/ml, or any range derivable therein.

If a liquid, gel, or semi-solid composition, the volume of the composition that is administered to the patient may be about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 microliters (μl) or milliliters (ml ), or any range derivable therein. In certain embodiments, the patient is administered up to about 10 ml of the composition.

The amount of an LtaS inhibitor compound of any of formula I through XII, or a pharmaceutically acceptable salt thereof, that is administered or taken by the patient may be based on the patient's weight (in kilograms). Therefore, in some embodiments, the patient is administered or takes a dose or multiple doses amounting to about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000 μg/kilogram (kg) or mg/kg, or any range derivable therein.

The composition may be administered to (or taken by) the patient 1, 2, 3, 4, 5, 6, 7, 8, 9, 10, 11, 12, 13, 14, 15, 16, 17, 18, 19, 20 or more times, or any range derivable therein, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months, or any range derivable therein. It is specifically contemplated that the composition may be administered once daily, twice daily, three times daily, four times daily, five times daily, or six times daily (or any range derivable therein) and/or as needed to the patient. Alternatively, the composition may be administered every 2, 4, 6, 8, 12 or 24 hours (or any range derivable therein) to or by the patient. In some embodiments, the patient is administered the composition for a certain period of time or with a certain number of doses after experiencing symptoms of a pathogenic bacterial infection.

In a further aspect the patient can be at risk for Staphylococcus infection. In another embodiment the patient can be at risk for Streptococcus infection. In additional embodiments the patient is at risk for a pathogenic bacterial infection, including infection by a Gram-positive bacteria. In still further aspects, the patient is at risk for pneumonia.

Certain embodiments are directed to methods where the patient is immune deficient, is immune-compromised, is hospitalized, is undergoing an invasive medical procedure, is infected with influenza virus or is on a respirator.

In still a further aspect the patient has a Staphylococcus infection, which includes but is not limited to pneumonia, sepsis, bacteremia, corneal infection, skin infection, infection of the central nervous system, or toxic shock syndrome.

In certain aspects the methods can further comprise the step of monitoring the patient for a Gram-positive bacterial infection within a week of first administering an LtaS inhibitor that comprises a compound of any of formula I through XII, or a pharmaceutically acceptable salt thereof.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 Structures of LtAs inhibitors and structural elements common to inhibitor structures.

FIGS. 2A-2I. Growth inhibitor 1771 targets the LTA biosynthesis pathway. (A) S. aureus cultures were grown in medium supplemented with either 1% DMSO (control) or two sub-lethal concentrations of compound for 1 hour and the optical density at 600 nm (OD600 nm) was recorded. (B-C) Cultures shown in (A) were normalized to the same density and cells were lysed to prepare extracts that were run on SDS-PAGE for visualization of total protein by Coomassie stain (B, left panel) or for immunoblot analyses with antibodies against LTA (B, right panel) or LtaS (C, top panel) and SrtA (C, lower panel). Native S. aureus LtaS is detected as both full-length and processed proteins (70 and 49 kDa, respectively). (D-I) Recombinant S. aureus LtaSSA (DEF) or B. anthracis LtaS2BA (GHI) were expressed in E. coli and synthesis of polyglycerolphosphate was observed by immunoblot. E. coli cultures expressing (+) or not (−) LtaSSA (D) or LtaS2BA on a plasmid (G) were grown in medium supplemented with either 1% DMSO (−) or 200 μM 1771 (+). Culture density measurements were used to normalize cell lysates that were separated by SDS-PAGE and visualized by Coomassie (E and H, left panels) or transferred for immunoblot analyses. Blots were probed with antibodies against LTA (E and H, right panels) and LtaS (F, I). Recombinant LtaSSA is detected as a double band like the native protein produced in S. aureus whereas only processed LtaS2BA is detected in E. coli extracts. Molecular weight markers are indicated in kDa.

FIG. 3. Identification of ltaS homologues in the databank.

FIG. 4A-4E. LTA inhibitor treatment affects appearance and morphology of Gram-positive cocci and bacilli. (A-D) Scanning electron micrograph of bacterial culture grown without or with inhibitor. Bacteria were cultured in BHI medium supplemented with either 1% DMSO (−inhibitor) or sublethal amounts of 1771 (+inhibitor) as follows: (A) S. aureus RN4220 cells ±30 μM compound 1771 scanned using a magnification of 10,000 (top) and 80,000 (bottom); (B) E. faecalis V583 cells ±25 μM compound 1771 at a magnification of 10,000 (top) and 24,000 (bottom); (C) E. faecium TX0016 cells ±20 μM compound 1771 at a magnification of 10,000 (top) and 20,000 (bottom); (D) B. anthracis Sterne cells ±5 μM compound 1771 at a magnification of 5,000 (top) or 10,000 (bottom). (E) Thin-section transmission electron micrographs of S. aureus reveal a thickening of the envelope with visible deformations in the presence of inhibitor. Samples for thin-section transmission electron microscopy were prepared from S. aureus RN4220 colonies grown on BHI agar with 0, 30 or 40 μM inhibitor. The top panels show electron micrographs of staphylococci in the midst of cell division. Brackets in the upper microgaphs indicate the positions of enlarged image sections shown below. M: plasma membrane; P: peptidoglycan layer containing teichoic acids. Scale bars are indicated at the bottom left of each micrograph.

FIG. 5. Inhibitor 1771 disrupts the interaction between LtaS and phosphatidylglycerol in vitro. Elution profile of phosphatidylglycerol using size-exclusion HPLC. Size exclusion HPLC of 2 nmol eLtaS or SrtA on BioBasic SEC300 column pre-equilibrated with 20 nmol nitro-benzoxadiazole PG containing chains of 16 carbon atoms (NBD-PGC16) reveals elution of the NBD-PGC16.eLtaS complex with absorbance at 460 nm (solid line), but not formation of a NBD-PGC16.SrtA complex (line designated with diamonds). Inclusion of 200 μM compound 1771 in the mobile HPLC phase abolished the elution of NBD-PGC16.eLtaS complex (dashed line).

FIG. 6A-6D. Inhibitory features of compound 1771. (A) Three-dimensional models showing the molecular hydrophobicity potential of phosphatidylglycerol (left) and compound 1771 (right). Models were generated using Galaxy 3D Structure Generator v2011.02. Color coding for hydrophobic and hydrophilic areas are shown. (B) Structural formula of compound 1771 with the chemical designation 2-oxo-2-[(5-phenyl-1,3,4-oxadiazol-2-yl)amino]ethylnaphtho[2,1-b]furan-1-ylacetate. The naphthofuranyl group is indicated with a grey line and the acronym NF and the remainder of the molecule is referred as the R group. Growth inhibitory activity and structural formula of substructures with intact naphthofuranyl group (C) or intact R region (D). Inhibitory activities were measured using S. aureus RN4220 and are displayed as mean with standard deviations of three independent experiments. Dose-response graphs were calculated by fitting data with variable slope sigmoidal function using GraphPad Prism 5. Corresponding IC50 values are presented in Table 3.

FIG. 7. Compound 1771 delays disease in a mouse model of sepsis. Survival of cohorts of BALB/c mice (n=15) treated with saline (mock) or LTA inhibitor and infected with S. aureus Newman. Statistical significance was analyzed with the logrank test: mock vs. compound 1771: P <0.0001. Data are representative of two independent experiments.

FIG. 8A-8B. Quantitative analyses of bacilli chain length and envelope thickness upon incubation of bacteria with compound 1771. (A) Light microscopy images of vegetative bacilli of strain Sterne (WT) recovered from liquid cultures incubated without (−) or with 5 μM 1771 (+) were analyzed for chain length and compared to the previously characterized ltaS1/S2 double mutant of B. anthracis Sterne (micrographs not shown). Data are presented as box and whiskers plot (n=100). Statistical significance was analyzed with the Student's t test (unpaired, 2-tailed). All P values were smaller than 0.0001. (B) Electron micrographs (examples shown in FIG. 2) were analyzed for envelope thickness. Measurements are presented as box and whiskers plot (n=124). The line inside the box marks the median. Bars outside the box represent maximum and minimum values. S. aureus, E. faecalis and E. faecium were isolated from colonies grown on BHI agar without (−) or with 30 μM compound 1771 (+) and B. anthracis from colonies grown on BHI agar without (−) or with 8 μM compound 1771 (+). The unpaired t-test function of Prism 5 software was used to compare the means of cell envelope measurements of untreated (−) and compound-treated (+) bacteria. All two-tailed P values were found to be smaller than 0.0001.

FIG. 9. It is believed that LtaS uses phosphatidylglycerol as substrate to transfer the polar head group, glycerophosphate, to the growing chain of lipid-anchored polyglycerophosphate. The polymerization reaction releases diacylglycerol, which is recycled into the phospholipid metabolism by diacylglycerol kinase.

FIG. 10. Compound 1771 inhibits eLtaS binding to and cleavage of phosphatidylglycerol (PG) in vitro. Using NBD-PGC6 with 6 carbon acyl chains cleavage of 2 nmol substrate by 2 nmol eLtaS was detectable after 6 hours incubation at 37° C. Chloroform extraction separated non-hydrolyzed NBD-PGC6 (aqueous phase—AP) from the hydrophobic reaction product nitro-benzoxadiazole diacylglycerol (NBD-DAGC6), which segregated into the organic phase (OP). Both phases were analyzed by normal-phase HPLC on a diol column (solid line). Elution profiles were monitored by fluorescence (excitation at 460 nm, emission at 534 nm). Identity of peak fractions was confirmed by mass spectrometry. Addition of 100 μM compound 1771 blocked NBD-DAGC6 production by eLtaS (dashed line). A control reaction incubated for 6 hours without eLtaS did not contain detectable amounts of NBD-DAGC6 (line designated with diamonds).

FIG. 11A-11C. Selecting for S. aureus variants with increased resistance to compound 1771. (A) S. aureus RN4220 (2.4×107 CFU) was spread on LB agar and filter disks soaked with compound 1771 or streptomycin were placed on the agar surface prior to incubation for 16 hours and photography. Black arrows identify large colonies formed from antibiotic-resistant variants. (B) The mutation frequency of S. aureus RN4220 was determined on Mueller-Hinton agar plates containing a concentration gradient from 10 to 200 μM compound 1771. Large square plates (225 mm side length) were inoculated with 2.0×109 CFU and incubated at either 37° C. or 42° C. (example shown). Small, slow-growing colonies were observed only after 3-4 days incubation (white arrow. Three of these isolates were analyzed for compound compound 1771 MIC and IC50 values (Table 7). (C) S. aureus RN4220 (WT) and three isolates from compound 1771 gradient plates were grown in LB, lysed in a bead beater and cell extracts subjected to DS-PAGE and immunoblotting using a monoclonal antibody to detect LTA/polyglycerol-phosphate (a-LTA) and rabbit-polyclonal antibodies for LtaS (α-LtaS) and SrtA (α-StrA). The migratory positions of molecular weight markers (in kDA) are indicated. The coding sequences for the ltaS gene from S. aureus RN4220 (WT) and the three isolates were amplified by PCR and subjected to DNA sequence analysis; mutational changes in the ltaS gene were not detected.

FIG. 12A-2D Treatment with growth inhibitors 1650-C01 and 1650-I01 reduces abundance of LTA in S. aureus. Culture medium was supplemented with either 1% DMSO (control) or 1 μM inhibitor and inoculated with overnight culture of S. aureus RN4220. Optical density was measured after 2 hours incubation (A) and used to normalize cell lysates prepared for separation by SDS-PAGE. Gels were stained with Coomassie (B) and analyzed by immunoblotting with antibodies against LTA (C), LtaS and SrtA (D). LtaS is detected as full-length and processed protein (70 and 49 kDa, respectively). SrtA immunoblot is a control. Molecular weight standards are marked in kDa.

FIG. 13A-13F Growth inhibitors of S. aureus with structural similarity to 1771 disrupt LTA synthesis in E. coli expressing ltaS. (A) Structural formulas and predicted three-dimensional (3D) models of the inhibitors are shown. The chemical designation for 1650-C01 is 4-(N-benzyl-N-isopropylsulfamoyl)-N-(5-(3-methoxyphenyl)-1,3,4-oxadiazol-2-yl)benzamide, for 1650-I01 is N-(5-(3,5-dimethoxyphenyl)-1,3,4-oxadiazol-2-yl)-4-(N-methyl-N-henylsulfamoyl)benzamide and for 1650-M01 is 4-(2-methylpiperidin-1-ylsulfonyl)-N-(5-(thiophen-2-yl)-1,3,4-oxadiazol-2-yl)benzamide. The 3D models display hydrophobic and and hydrophilic areas in different shades (Galaxy 3D Structure Generator at www.molinspiration.com). (B) Growth inhibition of S. aureus USA300 (MRSA) was determined by adding compound to microcultures on 96-well plates and measuring optical density at 600 nm after 18-22 hours incubation at 37° C. Dose-response graphs were calculated from 3 independent experiments using variable slope sigmoidal function (GraphPad Prism 5). IC50 values derived from dose-response graphs and observed MIC values are shown. (C-F) Recombinant LtaS from S. aureus was expressed in E. coli synthesizing LTA. Cultures lacking LtaS (−) or expressing LtaS (+) were grown in medium supplemented with either 1% DMSO (−) or 200 μM compound (+). Culture density measurements (C) were used to normalize cell lysates for SDS-PAGE separation. Gels were stained with Coomassie (D) and analyzed by immunoblotting with antibodies against LTA (E) and LtaS (F). Recombinant LtaS is detected as full-length and processed protein (70 and 49 kDa, respectively). Molecular weight standards are marked in kDa.

FIG. 14A-14B Candidates from secondary screen that specifically inhibited growth of MRSA without affecting the growth of E. coli or viability of HL-60 cells

FIG. 15A-15D Molecular structures of the compounds listed in FIGS. 14A-14B.

### DETAILED DESCRIPTION OF THE INVENTION

### A. LIPOTEICHOIC ACID SYNTHESIS

Many Gram-positive bacteria incorporate zwitterionic lipoteichoic acid (LTA) into their cell wall. This polymer was first identified as the heterophile antigen of pneumococcus (Goebel et al. 1943) and its structure was elucidated by Baddiley (Baddiley 1968). Clearly, LTA plays an important role during host infection, and it has been recognized for a long time that LTA triggers innate immune responses and is a TLR ligand (Morath et al. 2005). However, its physiological function is not well understood perhaps because the genetic disruption of LTA biosynthesis could not be achieved until recently (Grundling & Schneewind, PNAS 2007 and Grundling & Schneewind, J. Bacteriol. 2007). Thus, LTA has been proposed to be broadly important for scavenging Mg²⁺ ion and targeting autolysins in the bacterial envelope, a function that is essential for the separation of dividing cells (Lambert et al. 1977 and Cleveland et al. 1975). LTA purified from Staphylococcus aureus and other species is composed of a polymer of glycerol phosphate linked to glycolipid, which provides for LTA anchoring in bacterial membranes (Baddiley 1968, Coley et al. 1972, Reichman & Grundling 2011, and Fischer 1990). The glycerol moieties can be modified at the 2′OH position with D-alanyl esters or N-acetylglucosamine (Nehaus & Baddiley 2003). These modifications are important for escape from innate immune defenses such as host antimicrobial peptides and they contribute to the integrity of the overall cell envelope by limiting the activity of autolysins (Nehaus & Baddiley 2003). The genes responsible for modification at the 2′OH position of polyglycerol have been identified and shown to be dispensable both for growth and LTA synthesis (Nehaus & Baddiley 2003). The genes involved in the assembly of the membrane anchor moiety for LTA have also been identified. In S. aureus for example, this moiety is composed of β-gentiobiosyldiacylglycerol[glucosyl-(1→6)-glucosyl-(1→3)-diacylglycerol(G1c₂-DAG)] (Duckworth et al, 1975). Three enzymatic steps are required for the synthesis of G1c₂-DAG and involve the enzymes PgcA (α-phosphoglucomutase), GtaB (UTP:α-glucose-1-phosphate uridyl transferase) and YpfP (glycosyl-transferase) (Grundling & Schneewind, J. Bacteriol. 2007, Jorasch et al. 2000, and Kiriukhin et al. 2001). Following its synthesis in the inner leaflet of the plasma membrane, the G1c₂-DAG glycolipid is moved across the membrane by LtaA a member of the major facilitator super-family of proteins (Grundling & Schneewind, J. Bacteriol. 2007). Glycolipid anchor mutants (ltaA, ypfP, pgcA and gtaB) lead to morphological alterations, including increase in cell size and aberrant cell shapes (Grundling & Schneewind, J. Bacteriol. 2007, Kiriukhin et al. 2001, Grundling et al. 2006) but they continue to multiply. In these mutants, the glycerol phosphate polymer remains tethered to the membrane by a terminal DAG residue, instead of Glc₂-DAG (Grundling & Schneewind, J. Bacteriol. 2007 and Kiriukhin et al. 2001). The last steps of LTA synthesis include the polymerization of polyglycerol phosphate and its transfer to G1c₂-DAG (Koch et al. 1984). Gründling and Schneewind showed that in S. aureus both steps are catalyzed by a polytopic membrane protein with a large extracellular domain annotated in the Pfam database as a sulfatase domain (pfam00884) (Grundling & Schneewind, PNAS 2007). The corresponding gene was named lipoteichoic acid synthase (ltaS). Genetic depletion or loss of ltaS were found to result in severe cell division defects in S. aureus, such phenotypes being exacerbated at higher temperatures (Grundling & Schneewind, PNAS 2007 and Oku et al. 2009). The characterization of proteins with pfam00884 domain in other Gram-positive bacteria confirmed that these sulfatases are indeed responsible for the polymerization and membrane tethering of glycerol phosphates (Baddiley 1968, Coley et al. 1972, Reichman & Grundling 2011). Importantly, ltaS mutants exhibit reduced viability, increased cell size and altered envelope morphology in all organisms where they have been examined (Wormann et al. 2011, Webb et al. 2009, Schirner et al. 2009, Garufi et al. 2012 and Corrigan et al. 2011).

Under physiological conditions, growth of S. aureus, B. anthracis, L. monocytogenes or B. subitilis cannot occur without ltaS expression and LTA synthesis (Grundling & Schneewind, PNAS 2007, Wormann et al. 2011, Webb et al. 2009, Schirner et al. 2009, Garufi et al. 2012). LtaS, a polytopic membrane protein with a C-terminal catalytic domain, is located on bacterial surfaces but absent from human or animal tissues. Thus, LtaS and LTA synthesis meet the key criteria for the development of new antibiotics (Grundling & Schneewind, PNAS 2007 and Projan 2004). Therefore, embodiments described herein target LtaS and the LTA synthesis pathway. In certain embodiments, the LTA synthesis pathway is directly targeted and affected so as to inhibit a Gram-positive bacteria that uses LtaS and/or the LTA synthesis pathway.

### B. TREATMENT METHODS AND COMPOSITIONS

Method and compositions include treatments for a disease or condition caused by a Gram-positive bacteria that employs the LTA synthesis pathway. An LtaS inhibitor that comprises a compound of any of formula I through XII, or a pharmaceutically acceptable salt thereof, can be given to treat a person infected with or exposed to Gram-positive bacteria or suspected of having been exposed to Gram-positive bacteria or at risk of developing a Gram-positive bacteria infection. Methods may be employed with respect to individuals who have tested positive for exposure to Gram-positive bacteria or who are deemed to be at risk for infection based on possible exposure.

In particular, there are methods of treatment for a Gram-positive bacterial infection, particularly infections associated with antibiotic-resistant Gram-positive bacteria. Moreover, methods concern treating any condition or disease that is caused by, perpetuated by, or promoted by a Gram-positive bacteria infection. Prevention and treatment methods concern inhibiting the bacteria, such as its growth, lifespan, toxicity, reproducibility, tolerance to antibiotics, etc.

In some embodiments, the treatment is administered in conjunction with Gram-positive bacteria antigens or antibodies that bind Gram-positive bacteria and/or their proteins and/or carbohydrates. Furthermore, in some examples, treatment comprises administration of other agents commonly used against bacterial infection, such as one or more antibiotics.

The compositions and related methods, particularly administration of an LtaS inhibitor that comprises a compound of any of formula I through XII, or a pharmaceutically acceptable salt thereof, or a compound that inhibits LTA or LtaS, may also be used in combination with the administration of traditional therapies. These include, but are not limited to, the administration of vaccines; anti-bacterial antibodies; or antibiotics such as streptomycin, ciprofloxacin, doxycycline, gentamycin, chloramphenicol, trimethoprim, sulfamethoxazole, ampicillin, tetracycline or various combinations of antibiotics.

In one aspect, it is contemplated that an LtaS inhibitor that comprises a compound of any of formula I through XII, or a pharmaceutically acceptable salt thereof, is used in conjunction with other antibacterial treatment. Alternatively, the therapy may precede or follow the other agent treatment by intervals ranging from minutes to weeks. In embodiments where the other agents and/or a proteins or polynucleotides are administered separately, one would generally ensure that a significant period of time did not expire between the time of each delivery, such that the agent and antigenic composition would still be able to exert an advantageously combined effect on the subject. In such instances, it is contemplated that one may administer both modalities within about 12-24 h of each other or within about 6-12 h of each other. In some situations, it may be desirable to extend the time period for administration significantly, where several days (2, 3, 4, 5, 6 or 7) to several weeks (1, 2, 3, 4, 5, 6, 7 or 8) lapse between the respective administrations.

Various combinations may be employed, for example antibiotic therapy is “A” and the LtaS inhibitor is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of the compositions of the present invention to a patient/subject will follow general protocols for the administration of such compounds, taking into account the toxicity, if any, of the composition, or other compositions described herein. It is expected that the treatment cycles would be repeated as necessary. It also is contemplated that various standard therapies, such as hydration, may be applied in combination with the described therapy.

### C. LTAS INHIBITORS

1. LtaS Inhibitors

In some embodiments, pharmaceutical compositions are administered to a subject. Different aspects involve administering an effective amount of a composition to a subject. In some embodiments, a composition comprising an LtaS inhibitor may be administered to the subject or patient to protect against or treat infection by one or more staphylococcus pathogens. Additionally, such compounds can be administered in combination with an antibiotic or another standard antibacterial therapy. Such compositions will generally be dissolved or dispersed in a pharmaceutically acceptable carrier or aqueous medium.

The active compounds or APIs described herein can be formulated for parenteral administration, e.g., formulated for injection via the intravenous, intramuscular, sub-cutaneous, or even intraperitoneal routes. The preparation of an aqueous composition that contains a compound or compounds that inhibit LtaS activity will be known to those of skill in the art in light of the present disclosure. Typically, such compositions can be prepared as injectables, either as liquid solutions or suspensions; solid forms suitable for use to prepare solutions or suspensions upon the addition of a liquid prior to injection can also be prepared; and, the preparations can also be emulsified. In addition to the compounds formulated for parenteral administration, other pharmaceutically acceptable forms include, e.g., aerosolizable, inhalable, or instillable formulations; tablets or other solids for oral administration; time release capsules; creams; lotions; mouthwashes; and the like. The preparation of an such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi. In certain embodiments the active ingredient is combined with a liquid for intravenous administration.

The carrier also can be a solvent or dispersion medium containing, e.g., water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle that contains the basic dispersion medium and the required other ingredients from those enumerated above. In the case of sterile powders for the preparation of sterile injectable solutions, methods of preparation are, e.g., vacuum-drying and freeze-drying techniques, which yield a powder of the active ingredient, plus any additional desired ingredient from a previously sterile-filtered solution thereof.

As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent.

Some variation in dosage will necessarily occur depending on the condition of the subject. The person responsible for administration will, in any event, determine the appropriate dose for the individual subject. An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the effects desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

Typically, for a human adult (weighing approximately 70 kilograms), from about 0.1 mg to about 3000 mg (including all values and ranges there between), or from about 5 mg to about 1000 mg (including all values and ranges there between), or from about 10 mg to about 100 mg (including all values and ranges there between), of a compound are administered. It is understood that these dosage ranges are by way of example only, and that administration can be adjusted depending on the factors known to the skilled artisan.

2. In Vitro, Ex Vivo, or In Vivo Administration

As used herein, the term in vitro administration refers to manipulations performed on cells removed from or outside of a subject, including, but not limited to cells in culture. The term ex vivo administration refers to cells which have been manipulated in vitro, and are subsequently administered to a subject. The term in vivo administration includes all manipulations performed within a subject. In certain aspects of the present invention, the compositions may be administered either in vitro, ex vivo, or in vivo.

3. Antibodies and Passive Immunization

Another aspect is the administration of other therapies or vaccines in conjunction with an LtaS inhibitor. Methods of administering immunoglobulins directed at bacterial antigens to a recipient to prevent a staphylococcal infection can be considered a passive vaccine. Another aspect includes the use of active vaccines against staphylococcal infection in conjunction with LtaS inhibitors. Certain therapeutic methods include the administration of a therapeutic immunoglobulin or an antigen to stimulate or induce production of an immune response in a subject. A method of preparing an immunoglobulin for use in prevention or treatment of staphylococcal infection comprises the steps of immunizing a recipient or donor with a vaccine and isolating immunoglobulin from the recipient or donor. In certain aspect an immunoglobulin can bind to a cell surface protein, a toxin or any other component of the bacterium that is surface exposed, including, but not limited to lipoproteins and carbohydrate constituents of the bacterial cell wall. In other aspects an antibody may bind to a bacterial antigen and inhibit some necessary bacterial function. A pharmaceutical composition comprising an immunoglobulin, with or without an LtaS inhibitor, and a pharmaceutically acceptable carrier can be used in the manufacture of a medicament for the treatment or prevention of staphylococcal disease. A method for treatment or prevention of staphylococcal infection comprising a step of administering to a patient an effective amount of the pharmaceutical preparation of the invention is a further aspect.

The antibodies can be isolated to the extent desired by well known techniques such as affinity chromatography (Harlow and Lane, 1988). Antibodies can include antiserum preparations from a variety of commonly used animals, e.g. goats, primates, donkeys, swine, horses, guinea pigs, rats or man.

Any immunoglobulin, whether directed at any bacterial antigen and used in accordance with the present invention can include whole antibodies, antibody fragments or subfragments. Antibodies can be whole immunoglobulins of any class (e.g., IgG, IgM, IgA, IgD or IgE), chimeric antibodies or hybrid antibodies with dual specificity to two or more antigens of the invention. They may also be fragments (e.g., F(ab′)2, Fab′, Fab, Fv and the like) including hybrid fragments. An immunoglobulin also includes natural, synthetic, or genetically engineered proteins that act like an antibody by binding to specific antigens to form a complex.

A vaccine can be administered to a recipient who then acts as a source of immunoglobulin, produced in response to challenge from the specific vaccine. A subject thus treated would donate plasma from which hyperimmune globulin would be obtained via conventional plasma fractionation methodology. The hyperimmune globulin would be administered to another subject in order to impart resistance against or treat staphylococcal infection. Hyperimmune globulins are particularly useful for treatment or prevention of staphylococcal disease in infants, immune compromised individuals, or where treatment is required and there is no time for the individual to produce antibodies in response to vaccination.

### Definitions

When used in the context of a chemical group, “hydrogen” means —H; “hydroxy” means —OH; “oxo” means ═O; “halo” means independently —F, —Cl, —Br or —I; “amino” means —NH2 (see below for definitions of groups containing the term amino, e.g., alkylamino); “hydroxyamino” means —NHOH; “nitro” means —NO2; imino means ═NH (see below for definitions of groups containing the term imino, e.g., alkylimino); “cyano” means —CN; “azido” means —N3; in a monovalent context “phosphate” means —OP(O)(OH)2 or a deprotonated form thereof; in a divalent context “phosphate” means —OP(O)(OH)O— or a deprotonated form thereof; “mercapto” means —SH; “thio” means ═S; “thioether” means —S—; “sulfonamido” means —NHS(O)2— (see below for definitions of groups containing the term sulfonamido, e.g., alkylsulfonamido); “sulfonyl” means —S(O)2— (see below for definitions of groups containing the term sulfonyl, e.g., alkylsulfonyl); “sulfinyl” means —S(O)— (see below for definitions of groups containing the term sulfinyl, e.g., alkylsulfinyl); and “silyl” means —SiH3 (see below for definitions of group(s) containing the term silyl, e.g., alkylsilyl).

The symbol “—” means a single bond, “═” means a double bond, and “ [symbol] ” means triple bond. The symbol “ [symbol] ” represents a single bond or a double bond. The symbol “ [symbol] ”, when drawn perpendicularly across a bond indicates a point of attachment of the group. It is noted that the point of attachment is typically only identified in this manner for larger groups in order to assist the reader in rapidly and unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the conformation is unknown (e.g., either R or S), the geometry is unknown (e.g., either E or Z) or the compound is present as mixture of conformation or geometries (e.g., a 50%/50% mixture).

When a group “R” is depicted as a “floating group” on a ring system, for example, in the formula:

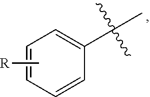

then R may replace any hydrogen atom attached to any of the ring atoms, including a depicted, implied, or expressly defined hydrogen, so long as a stable structure is formed.

When a group “R” is depicted as a “floating group” on a fused ring system, as for example in the formula:

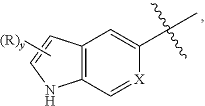

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

When y is 2 and “(R)y” is depicted as a floating group on a ring system having one or more ring atoms having two replaceable hydrogens, e.g., a saturated ring carbon, as for example in the formula:

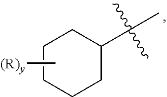

then each of the two R groups can reside on the same or a different ring atom. For example, when R is methyl and both R groups are attached to the same ring atom, a geminal dimethyl group results. Where specifically provided for, two R groups may be taken together to form a divalent group, such as one of the divalent groups further defined below. When such a divalent group is attached to the same ring atom, a spirocyclic ring structure will result.

In the case of a double-bonded R group (e.g., oxo, imino, thio, alkylidene, etc.), any pair of implicit or explicit hydrogen atoms attached to one ring atom can be replaced by the R group. This concept is exemplified below:

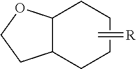

represents

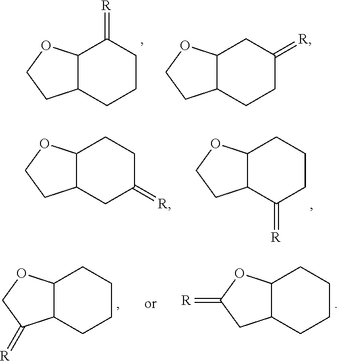

For the groups below, the following parenthetical subscripts further define the groups as follows: “(Cn)” defines the exact number (n) of carbon atoms in the group. “(C≦n)” defines the maximum number (n) of carbon atoms that can be in the group, with the minimum number of carbon atoms in such at least one, but otherwise as small as possible for the group in question, e.g., it is understood that the minimum number of carbon atoms in the group “alkenyl(C≦8)” is two. For example, “alkoxy(C≦10)” designates those alkoxy groups having from 1 to 10 carbon atoms (e.g., 1, 2, 3, 4, 5, 6, 7, 8, 9, or 10, or any range derivable therein (e.g., 3 to 10 carbon atoms). (Cn-n′) defines both the minimum (n) and maximum number (n′) of carbon atoms in the group. Similarly, “alkyl(C2-10)” designates those alkyl groups having from 2 to 10 carbon atoms (e.g., 2, 3, 4, 5, 6, 7, 8, 9, or 10, or any range derivable therein.

The term “alkyl” when used without the “substituted” modifier refers to a non-aromatic monovalent group with a saturated carbon atom as the point of attachment, a linear or branched, cyclo, cyclic or acyclic structure, no carbon-carbon double or triple bonds, and no atoms other than carbon and hydrogen. The groups, —CH₃(Me), —CH₂CH₃(Et), —CH₂CH₂CH₃(n-Pr), —CH(CH₃)₂(iso-Pr), —CH(CH₂)₂(cyclopropyl), —CH₂CH₂CH₂CH₃(n-Bu), —CH(CH₃)CH₂CH₃(sec-butyl), —CH₂CH(CH₃)₂(iso-butyl), —C(CH₃)₃(tert-butyl), —CH₂C(CH₃)₃(neo-pentyl), cyclobutyl, cyclopentyl, cyclohexyl, and cyclohexylmethyl are non-limiting examples of alkyl groups. The term “substituted alkyl” refers to a non-aromatic monovalent group with a saturated carbon atom as the point of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkyl groups: —CH₂OH, —CH₂Cl, —CH₂Br, —CH₂SH, —CF₃, —CH₂CN, —CH₂C(O)H, —CH₂C(O)OH, —CH₂C(O)OCH₃, —CH₂C(O)NH₂, —CH₂C(O)NHCH₃, —CH₂C(O)CH₃, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, —CH₂CF₃, —CH₂CH₂OC(O)CH₃, —CH₂CH₂NHCO₂C(CH₃)₃, and —CH₂Si(CH₃)₃.

The term “alkanediyl” when used without the “substituted” modifier refers to a non-aromatic divalent group, wherein the alkanediyl group is attached with two σ-bonds, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂—(methylene), —CH₂CH₂—, —CH₂C(CH₃)₂CH₂—, CH₂CH₂CH₂—, and

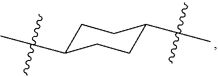 [SMILES: *C1CCC(*)CC1]

are non-limiting examples of alkanediyl groups. The term “substituted alkanediyl” refers to a non-aromatic monovalent group, wherein the alkynediyl group is attached with two σ-bonds, with one or two saturated carbon atom(s) as the point(s) of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkanediyl groups: —CH(F)—, —CF₂—, —CH(Cl)—, —CH(OH)—, —CH(OCH₃)—, and —CH₂CH(Cl)—.

The term “alkenyl” when used without the “substituted” modifier refers to a monovalent group with a nonaromatic carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂(vinyl), —CH═CHCH₃, —CH═CHCH₂CH₃, —CH₂CH═CH₂(allyl), —CH₂CH═CHCH₃, and —CH═CH—C₆H₅. The term “substituted alkenyl” refers to a monovalent group with a nonaromatic carbon atom as the point of attachment, at least one nonaromatic carbon-carbon double bond, no carbon-carbon triple bonds, a linear or branched, cyclo, cyclic or acyclic structure, and at least one atom independently selected from the group consisting of N, O, F, Cl, Br, I, Si, P, and S. The groups, —CH═CHF, —CH═CHCl and —CH═CHBr, are non-limiting examples of substituted alkenyl groups.

The term “alkenediyl” when used without the “substituted” modifier refers to a non-aromatic divalent group, wherein the alkenediyl group is attached with two σ-bonds, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, —CH═CH—, —CH═C(CH₃)CH₂—, —CH═CHCH₂—, and

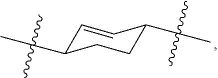 [SMILES: *C1C=CC(*)CC1]

are non-limiting examples of alkenediyl groups. The term “substituted alkenediyl” refers to a non-aromatic divalent group, wherein the alkenediyl group is attached with two σ-bonds, with two carbon atoms as points of attachment, a linear or branched, cyclo, cyclic or acyclic structure, at least one nonaromatic carbon-carbon double bond, no carbon-carbon triple bonds, and at least one atom independently selected from the group consisting of N, O, F, Cl, Br, I, Si, P, and S. The following groups are non-limiting examples of substituted alkenediyl groups: —CF═CH—, —C(OH)═CH—, and —CH₂CH═C(Cl)—.

The term “alkynyl” when used without the “substituted” modifier refers to a monovalent group with a nonaromatic carbon atom as the point of attachment, a linear or branched, cyclo, cyclic or acyclic structure, at least one carbon-carbon triple bond, and no atoms other than carbon and hydrogen. The groups, —C≡CH, —C≡CCH₃, —C≡CC₆H₅ and —CH₂C≡CCH₃, are non-limiting examples of alkynyl groups. The term “substituted alkynyl” refers to a monovalent group with a nonaromatic carbon atom as the point of attachment and at least one carbon-carbon triple bond, a linear or branched, cyclo, cyclic or acyclic structure, and at least one atom independently selected from the group consisting of N, O, F, Cl, Br, I, Si, P, and S. The group, —C≡CSi(CH₃)₃, is a non-limiting example of a substituted alkynyl group.

The term “alkynediyl” when used without the “substituted” modifier refers to a non-aromatic divalent group, wherein the alkynediyl group is attached with two σ-bonds, with two carbon atoms as points of attachment, a linear or branched, cyclo, cyclic or acyclic structure, at least one carbon-carbon triple bond, and no atoms other than carbon and hydrogen. The groups, —C≡C—, —C≡CCH₂—, and —C≡CCH(CH₃)— are non-limiting examples of alkynediyl groups. The term “substituted alkynediyl” refers to a non-aromatic divalent group, wherein the alkynediyl group is attached with two σ-bonds, with two carbon atoms as points of attachment, a linear or branched, cyclo, cyclic or acyclic structure, at least one carbon-carbon triple bond, and at least one atom independently selected from the group consisting of N, O, F, Cl, Br, I, Si, P, and S. The groups —C≡CCFH— and —C≡CHCH(Cl)— are non-limiting examples of substituted alkynediyl groups.

The term “aryl” when used without the “substituted” modifier refers to a monovalent group with an aromatic carbon atom as the point of attachment, said carbon atom forming part of one or more six-membered aromatic ring structure(s) wherein the ring atoms are all carbon, and wherein the monovalent group consists of no atoms other than carbon and hydrogen. Non-limiting examples of aryl groups include phenyl (Ph), methylphenyl, (dimethyl)phenyl, —C₆H₄CH₂CH₃(ethylphenyl), —C₆H₄CH₂CH₂CH₃(propylphenyl), C₆H₄CH(CH₃)₂, C₆H₄CH(CH₂)₂, —C₆H₃(CH₃)CH₂CH₃(methylethylphenyl), —C₆H₄CH═CH₂(vinylphenyl), —C₆H₄CH═CHCH₃, —C₆H₄C≡CH, —C₆H₄C≡CCH₃, naphthyl, and the monovalent group derived from biphenyl. The term “substituted aryl” refers to a monovalent group with an aromatic carbon atom as the point of attachment, said carbon atom forming part of one or more six-membered aromatic ring structure(s) wherein the ring atoms are all carbon, and wherein the monovalent group further has at least one atom independently selected from the group consisting of N, O, F, Cl, Br, I, Si, P, and S. Non-limiting examples of substituted aryl groups include the groups: —C₆H₄F, —C₆H₄Cl, —C₆H₄Br, —C₆H₄I, —C₆H₄OH, —C₆H₄OCH₃, —C₆H₄OCH₂CH₃, —C₆H₄OC(O)CH₃, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂NH₂, —C₆H₄CF₃, —C₆H₄CN, —C₆H₄CHO, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, and —C₆H₄CON(CH₃)₂.

The term “arenediyl” when used without the “substituted” modifier refers to a divalent group, wherein the arenediyl group is attached with two σ-bonds, with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. Non-limiting examples of arenediyl groups include:

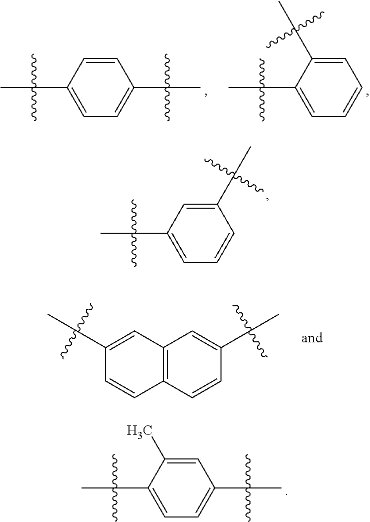

The term “substituted arenediyl” refers to a divalent group, wherein the arenediyl group is attached with two σ-bonds, with two aromatic carbon atoms as points of attachment, said carbon atoms forming part of one or more six-membered aromatic rings structure(s), wherein the ring atoms are carbon, and wherein the divalent group further has at least one atom independently selected from the group consisting of N, O, F, Cl, Br, I, Si, P, and S.

The term “aralkyl” when used without the “substituted” modifier refers to the monovalent group—alkanediyl—aryl, in which the terms alkanediyl and aryl are each used in a manner consistent with the definitions provided above. Non-limiting examples of aralkyls are: phenylmethyl (benzyl, Bn), 1-phenyl-ethyl, 2-phenyl-ethyl, indenyl and 2,3-dihydroindenyl, provided that indenyl and 2,3-dihydro-indenyl are only examples of aralkyl in so far as the point of attachment in each case is one of the saturated carbon atoms. When the term “aralkyl” is used with the “substituted” modifier, either one or both the alkanediyl and the aryl is substituted. Non-limiting examples of substituted aralkyls are: (3-chlorophenyl)-methyl, 2-oxo-2-phenyl-ethyl(phenylcarbonylmethyl), 2-chloro-2-phenyl-ethyl, chromanyl where the point of attachment is one of the saturated carbon atoms, and tetrahydroquinolinyl where the point of attachment is one of the saturated atoms.

The term “heteroaryl” when used without the “substituted” modifier refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group consists of no atoms other than carbon, hydrogen, aromatic nitrogen, aromatic oxygen and aromatic sulfur. Non-limiting examples of aryl groups include acridinyl, furanyl, imidazoimidazolyl, imidazopyrazolyl, imidazopyridinyl, imidazopyrimidinyl, indolyl, indazolinyl, methylpyridyl, oxazolyl, phenylimidazolyl, pyridyl, pyrrolyl, pyrimidyl, pyrazinyl, quinolyl, quinazolyl, quinoxalinyl, tetrahydroquinolinyl, thienyl, triazinyl, pyrrolopyridinyl, pyrrolopyrimidinyl, pyrrolopyrazinyl, pyrrolotriazinyl, pyrroloimidazolyl, chromenyl (where the point of attachment is one of the aromatic atoms), and chromanyl (where the point of attachment is one of the aromatic atoms). The term “substituted heteroaryl” refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group further has at least one atom independently selected from the group consisting of non-aromatic nitrogen, non-aromatic oxygen, non aromatic sulfur F, Cl, Br, I, Si, and P.

The term “heteroarenediyl” when used without the “substituted” modifier refers to a divalent group, wherein the heteroarenediyl group is attached with two σ-bonds, with two atoms, aromatic carbon atom and/or aromatic nitrogen, as the point of attachment, said carbon atom or nitrogen atom forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. Non-limiting examples of heteroarenediyl groups include:

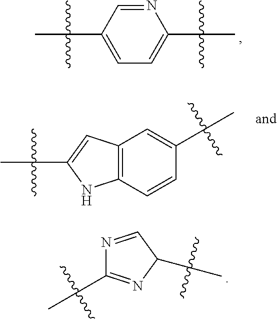

The term “substituted heteroarenediyl” refers to a divalent group, wherein the heteroarenediyl group is attached with two σ-bonds, with an aromatic carbon atom or nitrogen atom as points of attachment, said carbon atom or nitrogen atom forming part of one or more six-membered aromatic ring structure(s), wherein at least one of the ring atoms is nitrogen, oxygen or sulfur, and wherein the divalent group further has at least one atom independently selected from the group consisting of non-aromatic nitrogen, non-aromatic oxygen, non aromatic sulfur F, Cl, Br, I, Si, and P.

The term “heteroaralkyl” when used without the “substituted” modifier refers to the monovalent group—alkanediyl—heteroaryl, in which the terms alkanediyl and heteroaryl are each used in a manner consistent with the definitions provided above. Non-limiting examples of aralkyls are: pyridylmethyl, and thienylmethyl. When the term “heteroaralkyl” is used with the “substituted” modifier, either one or both the alkanediyl and the heteroaryl is substituted.

The term “acyl” when used without the “substituted” modifier refers to a monovalent group with a carbon atom of a carbonyl group as the point of attachment, further having a linear or branched, cyclo, cyclic or acyclic structure, further having no additional atoms that are not carbon or hydrogen, beyond the oxygen atom of the carbonyl group. The groups, —CHO, —C(O)CH₃(acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)C₆H₄CH₂CH₃, —COC₆H₃(CH₃)₂, and —C(O)CH₂C₆H₅, are non-limiting examples of acyl groups. The term “acyl” therefore encompasses, but is not limited to groups sometimes referred to as “alkyl carbonyl” and “aryl carbonyl” groups. The term “substituted acyl” refers to a monovalent group with a carbon atom of a carbonyl group as the point of attachment, further having a linear or branched, cyclo, cyclic or acyclic structure, further having at least one atom, in addition to the oxygen of the carbonyl group, independently selected from the group consisting of N, O, F, Cl, Br, I, Si, P, and S. The groups, —C(O)CH₂CF₃, —CO₂H (carboxyl), —CO₂CH₃(methylcarboxyl), —CO₂CH₂CH₃, —CO₂CH₂CH₂CH₃, —CO₂C₆H₅, —CO₂CH(CH₃)₂, —CO₂CH(CH₂)₂, —C(O)NH₂(carbamoyl), —C(O)NHCH₃, —C(O)NHCH₂CH₃, —CONHCH(CH₃)₂, —CONHCH(CH₂)₂, —CON(CH₃)₂, —CONHCH₂CF₃, —CO-pyridyl, —CO-imidazoyl, and —C(O)N₃, are non-limiting examples of substituted acyl groups. The term “substituted acyl” encompasses, but is not limited to, “heteroaryl carbonyl” groups.

The term “alkylidene” when used without the “substituted” modifier refers to the divalent group ═CRR', wherein the alkylidene group is attached with one σ-bond and one πbond, in which R and R′ are independently hydrogen, alkyl, or R and R′ are taken together to represent alkanediyl. Non-limiting examples of alkylidene groups include: ═CH₂, ═CH(CH₂CH₃), and ═C(CH₃)₂. The term “substituted alkylidene” refers to the group ═CRR', wherein the alkylidene group is attached with one σ-bond and one 90-bond, in which R and R′ are independently hydrogen, alkyl, substituted alkyl, or R and R′ are taken together to represent a substituted alkanediyl, provided that either one of R and R′ is a substituted alkyl or R and R′ are taken together to represent a substituted alkanediyl.

The term “alkoxy” when used without the “substituted” modifier refers to the group —OR, in which R is an alkyl, as that term is defined above. Non-limiting examples of alkoxy groups include: —OCH₃, —OCH₂CH₃, —OCH₂CH₂CH₃, —OCH(CH₃)₂, —OCH(CH₂)₂, —O-cyclopentyl, and —O-cyclohexyl. The term “substituted alkoxy” refers to the group —OR, in which R is a substituted alkyl, as that term is defined above. For example, —OCH₂CF₃ is a substituted alkoxy group.

The term “alkoxydiyl” when used without the “substituted” modifier refers to a non-aromatic divalent group, wherein the alkoxydiyl group is attached with two σ-bonds, with (a) two saturated carbon atoms as points of attachment, (b) one saturated carbon atom and one oxygen atom as points of attachment, or (c) two oxygen atoms as points of attachment, further having a linear or branched, cyclo, cyclic or acyclic structure, no carbon-carbon double or triple bonds in the group's backbone, further having no backbone atoms other than carbon or oxygen and having at least one of each of these atoms in the group's backbone, and no side chains comprising groups other than hydrogen or alkyl. The groups, —O—CH₂CH₂—, —CH₂—O—CH₂CH₂—, —O—CH₂CH₂—O— and —O—CH₂—O— are non-limiting examples of alkoxydiyl groups. The term “substituted alkanyloxydiyl” refers to a divalent group that is attached with two σ-bonds, with (a) two saturated carbon atoms as points of attachment, (b) one saturated carbon atom and one oxygen atom as points of attachment, or (c) two oxygen atoms as points of attachment, further having a linear or branched, cyclo, cyclic or acyclic structure, no carbon-carbon double or triple bonds, and at least one atom independently selected from the group consisting of N, F, Cl, Br, I, Si, P, and S, or having additional oxygen atoms beyond those in the group's backbone. The following groups are non-limiting example of a substituted alkoxydiyl groups: —O—CH₂C(OH)H—O— and —O—CH₂C(Cl)H—O—.

The terms “alkenyloxy”, “alkynyloxy”, “aryloxy”, “aralkoxy”, “heteroaryloxy”, “heteroaralkoxy” and “acyloxy”, when used without the “substituted” modifier, refers to groups, defined as —OR, in which R is alkenyl, alkynyl, aryl, aralkyl, heteroaryl, heteroaralkyl and acyl, respectively, as those terms are defined above. When any of the terms alkenyloxy, alkynyloxy, aryloxy, aralkyloxy and acyloxy is modified by “substituted,” it refers to the group —OR, in which R is substituted alkenyl, alkynyl, aryl, aralkyl, heteroaryl, heteroaralkyl and acyl, respectively.

The term “alkenyloxydiyl” when used without the “substituted” modifier refers to a divalent group that is nonaromatic prior to attachment, wherein the alkenyloxydiyl group is attached with two π-bonds, which may become aromatic upon attachment, with (a) two carbon atoms as points of attachment, (b) one carbon atom and one oxygen atom as points of attachment, or (c) two oxygen atoms as points of attachment, further having a linear or branched, cyclo, cyclic or acyclic structure, at least one carbon-carbon double bond that is non-aromatic at least prior to attachment, further having no backbone atoms other than carbon or oxygen and having at least one of each of these atoms in the group's backbone, and no side chains comprising groups other than hydrogen or alkyl. The groups, —O—CH═CH—, —O—CH═CHO— and —O—CH═CHCH₂— are non-limiting examples of alkenyloxydiyl groups. The term “substituted alkenyloxydiyl” refers to a divalent group that is nonaromatic prior to attachment, wherein the substituted alkenyloxydiyl group is attached with two σ-bonds, which may become aromatic upon attachment, with (a) two carbon atoms as points of attachment, (b) one carbon atom and one oxygen atom as points of attachment, or (c) two oxygen atoms as points of attachment, further having a linear or branched, cyclo, cyclic or acyclic structure, at least one carbon-carbon double bond that is non-aromatic at least prior to attachment and at least one atom independently selected from the group consisting of N, F, Cl, Br, I, Si, P, and S, or having additional oxygen atoms beyond those in the group's backbone. The following groups are non-limiting example of a substituted alkenyloxydiyl groups: —O—CH═C(OH)—O— and —O—CH═C(Cl)—O—.

The term “alkylamino” when used without the “substituted” modifier refers to the group —NHR, in which R is an alkyl, as that term is defined above. Non-limiting examples of alkylamino groups include: —NHCH₃, —NHCH₂CH₃, —NHCH₂CH₂CH₃, —NHCH(CH₃)₂, —NHCH(CH₂)₂, —NHCH₂CH₂CH₂CH₃, —NHCH(CH₃)CH₂CH₃, —NHCH₂CH(CH₃)₂, —NHC(CH₃)₃, —NH—cyclopentyl, and —NH—cyclohexyl. The term “substituted alkylamino” refers to the group —NHR, in which R is a substituted alkyl, as that term is defined above. For example, —NHCH₂CF₃ is a substituted alkylamino group.

The term “dialkylamino” when used without the “substituted” modifier refers to the group —NRR′, in which R and R′ can be the same or different alkyl groups, or R and R′ can be taken together to represent an alkanediyl having two or more saturated carbon atoms, at least two of which are attached to the nitrogen atom. Non-limiting examples of dialkylamino groups include: —NHC(CH₃)₃, —N(CH₃)CH₂CH₃, —N(CH₂CH₃)₂, N-pyrrolidinyl, and N-piperidinyl. The term “substituted dialkylamino” refers to the group —NRR′, in which R and R′ can be the same or different substituted alkyl groups, one of R or R′ is an alkyl and the other is a substituted alkyl, or R and R′ can be taken together to represent a substituted alkanediyl with two or more saturated carbon atoms, at least two of which are attached to the nitrogen atom.

The terms “alkoxyamino”, “alkenylamino”, “alkynylamino”, “arylamino”, “aralkylamino”, “heteroarylamino”, “heteroaralkylamino”, and “alkylsulfonylamino” when used without the “substituted” modifier, refers to groups, defined as —NHR, in which R is alkoxy, alkenyl, alkynyl, aryl, aralkyl, heteroaryl, heteroaralkyl and alkylsulfonyl, respectively, as those terms are defined above. A non-limiting example of an arylamino group is —NHC₆H₅. When any of the terms alkoxyamino, alkenylamino, alkynylamino, arylamino, aralkylamino, heteroarylamino, heteroaralkylamino and alkylsulfonylamino is modified by “substituted,” it refers to the group —NHR, in which R is substituted alkoxy, alkenyl, alkynyl, aryl, aralkyl, heteroaryl, heteroaralkyl and alkylsulfonyl, respectively.

The term “amido” (acylamino), when used without the “substituted” modifier, refers to the group —NHR, in which R is acyl, as that term is defined above. A non-limiting example of an acylamino group is —NHC(O)CH₃. When the term amido is used with the “substituted” modifier, it refers to groups, defined as —NHR, in which R is substituted acyl, as that term is defined above. The groups —NHC(O)OCH₃ and —NHC(O)NHCH₃ are non-limiting examples of substituted amido groups.

The term “alkylaminodiyl” when used without the “substituted” modifier refers to a non-aromatic divalent group, wherein the alkylaminodiyl group is attached with two σ-bonds, with (a) two saturated carbon atoms as points of attachment, (b) one saturated carbon atom and one nitrogen atom as points of attachment, or (c) two nitrogen atoms as points of attachment, further having a linear or branched, cyclo, cyclic or acyclic structure, no double or triple bonds in the group's backbone, further having no backbone atoms other than carbon or nitrogen and having at least one of each of these atoms in the group's backbone, and no side chains comprising groups other than hydrogen or alkyl. The groups, —NH—CH₂CH₂—, —CH₂—NH—CH₂CH₂—, —NH—CH₂CH₂—NH— and —NH—CH₂—NH— are non-limiting examples of alkylaminodiyl groups. The term “substituted alkylaminodiyl” refers to a divalent group, wherein the substituted alkylaminodiyl group is attached with two σ-bonds, with (a) two saturated carbon atoms as points of attachment, (b) one saturated carbon atom and one nitrogen atom as points of attachment, or (c) two nitrogen atoms as points of attachment, further having a linear or branched, cyclo, cyclic or acyclic structure, no carbon-carbon double or triple bonds in the group's backbone, and at least one atom independently selected from the group consisting of O, F, Cl, Br, I, Si, P, and S, or having additional nitrogen atom beyond those in the group's backbone. The following groups are non-limiting example of a substituted alkylaminodiyl groups: —NH—CH₂C(OH)H—NH—and —NH—CH₂C(Cl)H—CH₂—.

The term “alkenylaminodiyl” when used without the “substituted” modifier refers to a divalent group that is nonaromatic prior to attachment, wherein the alkenylaminodiyl group is attached with two σ-bonds, which may become aromatic upon attachment, with (a) two carbon atoms as points of attachment, (b) one carbon atom and one nitrogen atom as points of attachment, or (c) two nitrogen atoms as points of attachment, further having a linear or branched, cyclo, cyclic or acyclic structure, at least one carbon-carbon double bond or carbon-nitrogen double that is non-aromatic at least prior to attachment, further having no backbone atoms other than carbon or nitrogen, and no side chains comprising groups other than hydrogen or alkyl. The groups, —NH'CH═CH—, —NH—CH═N— and —NH—CH═CH—NH— are non-limiting examples of alkenylaminodiyl groups. The term “substituted alkenylaminodiyl” refers to a divalent group that is nonaromatic prior to attachment, wherein the substituted alkenylaminodiyl group is attached with two σ-bonds, which may become aromatic upon attachment, with (a) two carbon atoms as points of attachment, (b) one carbon atom and one nitrogen atom as points of attachment, or (c) two nitrogen atoms as points of attachment, further having a linear or branched, cyclo, cyclic or acyclic structure, at least one carbon-carbon double bond or carbon nitrogen double bond that is non-aromatic at least prior to attachment and at least one atom independently selected from the group consisting of O, F, Cl, Br, I, Si, P, and S, or having additional nitrogen atoms beyond those in the group's backbone. The following groups are non-limiting example of a substituted alkenylaminodiyl groups: —NH—CH═C(OH)—CH₂— and —N═CHC(Cl)H—.

The term “alkenylaminooxydiyl” when used without the “substituted” modifier refers to a divalent group, wherein the alkenylaminooxydiyl group is attached with two σ-bonds, which may become aromatic upon attachment, with two atoms selected from the group consisting of carbon, oxygen and nitrogen as points of attachment, further having a linear or branched, cyclo, cyclic or acyclic structure, at least one carbon-carbon double bond, carbon-nitrogen double, or nitrogen-nitrogen double bond that is non-aromatic at least prior to attachment, further having no backbone atoms other than carbon nitrogen or oxygen and having at least one of each of these three atoms in the backbone, and no side chains comprising groups other than hydrogen or alkyl. The group —O—CH═N—, is a non-limiting example of an alkenylaminooxydiyl group. The term “substituted alkenylaminooxydiyl” refers to a divalent group that is attached with two π-bonds, which may become aromatic upon attachment with two atoms selected from the group consisting of carbon, oxygen and nitrogen as points of attachment, further having a linear or branched, cyclo, cyclic or acyclic structure, at least one carbon-carbon double bond or carbon nitrogen double bond that is non-aromatic at least prior to attachment and at least one atom independently selected from the group consisting of F, Cl, Br, I, Si, P, and S, or having one or more additional nitrogen and/or oxygen atoms beyond those in the group's backbone. The following groups are non-limiting example of a substituted alkenylaminooxydiyl groups: —NH—CH═C(OH)—O— and —N═CHC(Cl)H—O—.

The term “alkylimino” when used without the “substituted” modifier refers to the group ═NR, wherein the alkylimino group is attached with one σ-bond and one π-bond, in which R is an alkyl, as that term is defined above. Non-limiting examples of alkylimino groups include: ═NCH₃, ═NCH₂CH₃ and ═N-cyclohexyl. The term “substituted alkylimino” refers to the group ═NR, wherein the alkylimino group is attached with one σ-bond and one π-bond, in which R is a substituted alkyl, as that term is defined above. For example, ═NCH₂CF₃ is a substituted alkylimino group.

Similarly, the terms “alkenylimino”, “alkynylimino”, “arylimino”, “aralkylimino”, “heteroarylimino”, “heteroaralkylimino” and “acylimino”, when used without the “substituted” modifier, refers to groups, defined as ═NR, wherein the alkylimino group is attached with one σ-bond and one π-bond, in which R is alkenyl, alkynyl, aryl, aralkyl, heteroaryl, heteroaralkyl and acyl, respectively, as those terms are defined above. When any of the terms alkenylimino, alkynylimino, arylimino, aralkylimino and acylimino is modified by “substituted,” it refers to the group ═NR, wherein the alkylimino group is attached with one σ-bond and one π-bond, in which R is substituted alkenyl, alkynyl, aryl, aralkyl, heteroaryl, heteroaralkyl and acyl, respectively.

The term “fluoroalkyl” when used without the “substituted” modifier refers to an alkyl, as that term is defined above, in which one or more fluorines have been substituted for hydrogens. The groups, —CH₂F, —CF₂H, —CF₃, and —CH₂CF₃ are non-limiting examples of fluoroalkyl groups. The term “substituted fluoroalkyl” refers to a non-aromatic monovalent group with a saturated carbon atom as the point of attachment, a linear or branched, cyclo, cyclic or acyclic structure, at least one fluorine atom, no carbon-carbon double or triple bonds, and at least one atom independently selected from the group consisting of N, O, Cl, Br, I, Si, P, and S. The following group is a non-limiting example of a substituted fluoroalkyl: —CFHOH.

The term “alkylphosphate” when used without the “substituted” modifier refers to the group —OP(O)(OH)(OR), in which R is an alkyl, as that term is defined above. Non-limiting examples of alkylphosphate groups include: —OP(O)(OH)(OMe) and —OP(O)(OH)(OEt). The term “substituted alkylphosphate” refers to the group —OP(O)(OH)(OR), in which R is a substituted alkyl, as that term is defined above.

The term “dialkylphosphate” when used without the “substituted” modifier refers to the group —OP(O)(OR)(OR′), in which R and R′ can be the same or different alkyl groups, or R and R′ can be taken together to represent an alkanediyl having two or more saturated carbon atoms, at least two of which are attached via the oxygen atoms to the phosphorus atom. Non-limiting examples of dialkylphosphate groups include: —OP(O)(OMe)₂, —OP(O)(OEt)(OMe) and —OP(O)(OEt)₂. The term “substituted dialkylphosphate” refers to the group —OP(O)(OR)(OR′), in which R and R′ can be the same or different substituted alkyl groups, one of R or R′ is an alkyl and the other is a substituted alkyl, or R and R′ can be taken together to represent a substituted alkanediyl with two or more saturated carbon atoms, at least two of which are attached via the oxygen atoms to the phosphorous.

The term “alkylthio” when used without the “substituted” modifier refers to the group —SR, in which R is an alkyl, as that term is defined above. Non-limiting examples of alkylthio groups include: —SCH₃, —SCH₂CH₃, —SCH₂CH₂CH₃, —SCH(CH₃)₂, —SCH(CH₂)₂, —S-cyclopentyl, and —S-cyclohexyl. The term “substituted alkylthio” refers to the group —SR, in which R is a substituted alkyl, as that term is defined above. For example, —SCH₂CF₃ is a substituted alkylthio group.

Similarly, the terms “alkenylthio”, “alkynylthio”, “arylthio”, “aralkylthio”, “heteroarylthio”, “heteroaralkylthio”, and “acylthio”, when used without the “substituted” modifier, refers to groups, defined as —SR, in which R is alkenyl, alkynyl, aryl, aralkyl, heteroaryl, heteroaralkyl and acyl, respectively, as those terms are defined above. When any of the terms alkenylthio, alkynylthio, arylthio, aralkylthio, heteroarylthio, heteroaralkylthio, and acylthio is modified by “substituted,” it refers to the group —SR, in which R is substituted alkenyl, alkynyl, aryl, aralkyl, heteroaryl, heteroaralkyl and acyl, respectively.

The term “thioacyl” when used without the “substituted” modifier refers to a monovalent group with a carbon atom of a thiocarbonyl group as the point of attachment, further having a linear or branched, cyclo, cyclic or acyclic structure, further having no additional atoms that are not carbon or hydrogen, beyond the sulfur atom of the carbonyl group. The groups, —CHS, —C(S)CH₃, —C(S)CH₂CH₃, —C(S)CH₂CH₂CH₃, —C(S)CH(CH₃)₂, —C(S)CH(CH₂)₂, —C(S)C₆H₅, —C(S)C₆H₄CH₃, —C(S)C₆H₄CH₂CH₃, —C(S)C₆H₃(CH₃)₂, and —C(S)CH₂C₆H₅, are non-limiting examples of thioacyl groups. The term “thioacyl” therefore encompasses, but is not limited to, groups sometimes referred to as “alkyl thiocarbonyl” and “aryl thiocarbonyl” groups. The term “substituted thioacyl” refers to a radical with a carbon atom as the point of attachment, the carbon atom being part of a thiocarbonyl group, further having a linear or branched, cyclo, cyclic or acyclic structure, further having at least one atom, in addition to the sulfur atom of the carbonyl group, independently selected from the group consisting of N, O, F, Cl, Br, I, Si, P, and S. The groups, —C(S)CH₂CF₃, —C(S)O₂H, —C(S)OCH₃, —C(S)OCH₂CH₃, —C(S)OCH₂CH₂CH₃, —C(S)OC₆H₅, —C(S)OCH(CH₃)₂, —C(S)OCH(CH₂)₂, —C(S)NH₂, and —C(S)NHCH₃, are non-limiting examples of substituted thioacyl groups. The term “substituted thioacyl” encompasses, but is not limited to, “heteroaryl thiocarbonyl” groups.

The term “alkylsulfonyl” when used without the “substituted” modifier refers to the group —S(O)₂R, in which R is an alkyl, as that term is defined above. Non-limiting examples of alkylsulfonyl groups include: —S(O)₂CH₃, —S(O)₂CH₂CH₃, —S(O)₂CH₂CH₂CH₃, —S(O)₂CH(CH₃)₂, —S(O)₂CH(CH₂)₂, —S(O)₂-cyclopentyl, and —S(O)₂-cyclohexyl. The term “substituted alkylsulfonyl” refers to the group —S(O)₂R, in which R is a substituted alkyl, as that term is defined above. For example, —S(O)₂CH₂CF₃ is a substituted alkylsulfonyl group.

Similarly, the terms “alkenylsulfonyl”, “alkynylsulfonyl”, “arylsulfonyl”, “aralkylsulfonyl”, “heteroarylsulfonyl”, and “heteroaralkylsulfonyl” when used without the “substituted” modifier, refers to groups, defined as —S(O)₂R, in which R is alkenyl, alkynyl, aryl, aralkyl, heteroaryl, and heteroaralkyl, respectively, as those terms are defined above. When any of the terms alkenylsulfonyl, alkynylsulfonyl, arylsulfonyl, aralkylsulfonyl, heteroarylsulfonyl, and heteroaralkylsulfonyl is modified by “substituted,” it refers to the group —S(O)₂R, in which R is substituted alkenyl, alkynyl, aryl, aralkyl, heteroaryl and heteroaralkyl, respectively.

The term “alkylsulfinyl” when used without the “substituted” modifier refers to the group —S(O)R, in which R is an alkyl, as that term is defined above. Non-limiting examples of alkylsulfinyl groups include: —S(O)CH₃, —S(O)CH₂CH₃, —S(O)CH₂CH₂CH₃, —S(O)CH(CH₃)₂, —S(O)CH(CH₂)₂, —S(O)-cyclopentyl, and —S(O)-cyclohexyl. The term “substituted alkylsulfinyl” refers to the group —S(O)R, in which R is a substituted alkyl, as that term is defined above. For example, —S(O)CH₂CF₃ is a substituted alkylsulfinyl group.

Similarly, the terms “alkenylsulfinyl”, “alkynylsulfinyl”, “arylsulfinyl”, “aralkylsulfinyl”, “heteroarylsulfinyl”, and “heteroaralkylsulfinyl” when used without the “substituted” modifier, refers to groups, defined as —S(O)R, in which R is alkenyl, alkynyl, aryl, aralkyl, heteroaryl, and heteroaralkyl, respectively, as those terms are defined above. When any of the terms alkenylsulfinyl, alkynylsulfinyl, arylsulfinyl, aralkylsulfinyl, heteroarylsulfinyl, and heteroaralkylsulfinyl is modified by “substituted,” it refers to the group —S(O)R, in which R is substituted alkenyl, alkynyl, aryl, aralkyl, heteroaryl and heteroaralkyl, respectively.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Similarly, it is contemplated that one or more carbon atom(s) of a compound of the present invention may be replaced by a silicon atom(s). Furthermore, it is contemplated that one or more oxygen atom(s) of a compound of the present invention may be replaced by a sulfur or selenium atom(s).

A single dashed line between two atoms indicates an optional bond. The bond may not be present at all, it may be present as a single bond, or it may be present as a double bound. If an atom is only connected to dashed lines, then the atom itself is optional. It may be present or it may not be present.

A bond shown as a combination of a solid and a dashed line indicates that the bond is either a single bond or a double bond. Thus, for example, the structure

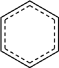

includes the structures

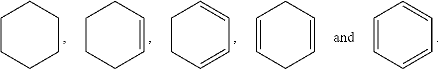

As will be understood by a person of skill in the art, no one such ring atom forms part of more than one double bond.

Any undefined valency on an atom of a structure shown in this application implicitly represents a hydrogen atom bonded to the atom.

As used herein, a “chiral auxiliary” refers to a removable chiral group that is capable of influencing the stereoselectivity of a reaction. Persons of skill in the art are familiar with such compounds, and many are commercially available.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (2002).

As used herein, “predominantly one enantiomer” means that a compound contains at least about 85% of one enantiomer, or more preferably at least about 90% of one enantiomer, or even more preferably at least about 95% of one enantiomer, or most preferably at least about 99% of one enantiomer. Similarly, the phrase “substantially free from other optical isomers” means that the composition contains at most about 15% of another enantiomer or diastereomer, more preferably at most about 10% of another enantiomer or diastereomer, even more preferably at most about 5% of another enantiomer or diastereomer, and most preferably at most about 1% of another enantiomer or diastereomer.

“Prevention” or “preventing” includes: (1) inhibiting the onset of a Gram-positive infection or infection-causing disease in a subject or patient which may be at risk and/or predisposed to the infection or disease but does not yet experience or display any or all of the pathology or symptomatology of such, and/or (2) slowing the onset of the pathology or symptomatology of an infection or disease in a subject or patient which may be at risk and/or predisposed to the infection or disease but does not yet experience or display any or all of the pathology or symptomatology of it.

“Prodrug” means a compound that is convertible in vivo metabolically into an inhibitor according to embodiments discussed herein. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂—]ₙ—, the repeat unit is —CH₂CH₂—. The subscript “n” denotes the degree of polymerisation, that is, the number of repeat units linked together. When the value for “n” is left undefined, it simply designates repetition of the formula within the brackets as well as the polymeric nature of the material. The concept of a repeat unit applies equally to where the connectivity between the repeat units extends three dimensionally, such as in metal organic frameworks, cross-linked polymers, thermosetting polymers, etc.

The term “saturated” when referring to an atom means that the atom is connected to other atoms only by means of single bonds.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers.

It is contemplated that for any stereocenter or axis of chirality for which stereochemistry has not been defined, that stereocenter or axis of chirality can be present in its R form, S form, or as a mixture of the R and S forms, including racemic and non-racemic mixtures. Compounds employed in methods of the invention may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S- or the R-configuration, as defined by the IUPAC 1974 Recommendations. Compounds may be of the D- or L- form, for example. It is well known in the art how to prepare and isolate such optically active forms. For example, mixtures of stereoisomers may be separated by standard techniques including, but not limited to, resolution of racemic form, normal, reverse-phase, and chiral chromatography, preferential salt formation, recrystallization, and the like, or by chiral synthesis either from chiral starting materials or by deliberate synthesis of target chiral centers.

“Substituent convertible to hydrogen in vivo” means any group that is convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydro-pyranyl, diphenylphosphinyl, and the like. Examples of acyl groups include formyl, acetyl, trifluoroacetyl, and the like. Examples of groups having an oxycarbonyl group include ethoxycarbonyl, tert-butoxycarbonyl (—C(O)OC(CH₃)₃), benzyloxycarbonyl, p-methoxybenzyloxycarbonyl, vinyloxycarbonyl, β-(p-toluenesulfonyl)ethoxycarbonyl, and the like. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), Ile (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable amino acid residues also include amino acid residues that are protected with a protecting group. Examples of suitable protecting groups include those typically employed in peptide synthesis, including acyl groups (such as formyl and acetyl), arylmethyloxycarbonyl groups (such as benzyloxycarbonyl and p-nitrobenzyloxycarbonyl), tert-butoxycarbonyl groups (—C(O)OC(CH₃)₃), and the like. Suitable peptide residues include peptide residues comprising two to five, and optionally amino acid residues. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethyloxycarbonyl groups (such as benzyloxycarbonyl and p-nitrobenzyloxycarbonyl), tert-butoxycarbonyl groups (—C(O)OC(CH₃)₃), and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and haloethoxycarbonyl groups (such as β,β,β-trichloroethoxycarbonyl and (β-iodoethoxycarbonyl).

“Therapeutically effective amount” or “pharmaceutically effective amount” means that amount which, when administered to a subject or patient for treating the infection or a disease or condition caused by the infection, is sufficient to effect such treatment.

“Treatment” or “treating” includes (1) inhibiting a Gram-positive infection or a disease or condition that is caused by the Gram-positive infection in a subject or patient experiencing or displaying the pathology or symptomatology of the infection, disease, or condition (e.g., arresting further development of the pathology and/or symptomatology), (2) ameliorating the infection, disease, or condition in a subject or patient that is experiencing or displaying the pathology or symptomatology of the disease (e.g., reversing the pathology and/or symptomatology), and/or (3) effecting any measurable decrease in the infection, disease, or condition in a subject or patient that is experiencing or displaying the relevant pathology or symptomatology.

As used herein, the term “water soluble” means that the compound dissolves in water at least to the extent of 0.010 mole/liter or is classified as soluble according to literature precedence.

The above definitions supersede any conflicting definition in any of the reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### D. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of certain embodiments, are provided as an example, and are not intended as limitations on the scope of the invention. Changes therein and other uses which are encompassed within the spirit of the invention as defined by the scope of the claims will occur to those skilled in the art.

### Example 1

### Material and Methods for Examples 2-7

### High Throughput Screen

The antibiotic sensitive strain S. aureus RN4220 was used to screen for small molecule growth inhibitors in a high throughput-screening assay using 384-well microplates. Cation-adjusted Hinton Mueller II broth supplemented with 0.005% Tween-80 was inoculated with a single colony of S. aureus RN4220. Following overnight incubation, the culture was diluted 1:100 in fresh medium and incubation was continued until the bacterial suspension reached an optical density at 600 nm (OD₆₀₀) of 0.6 corresponding to a bacterial titer of 5×10⁸ colony forming units (CFU) per ml. An aliquot of this culture was diluted 1:400 with fresh medium and stored on ice until it could be dispensed in 384-well plates. Small-scale experiments were conducted to determine the Z′-factor (1-[(3SD⁻)+(3SD⁺)/(Ave⁻)−(Ave⁺)]) used commonly for quality assessment of raw data sets generated in high-throughput screening. In this formula, (SD^{+/-}) and (Ave^{+/-}) represent standard deviation and average of positive and negative controls. Four 384-well plates were loaded with 25 μl medium per well. Half the wells were supplemented with 4 nmol chloramphenicol each (positive controls), whereas the remaining wells were not (negative controls). Each well was inoculated with 25 μl aliquot of the culture kept on ice (approximately 3.125×10⁴ CFU). The OD₆₀₀ in each well was recorded after 20 hours incubation. Z′ values varied between 0.72 and 0.84. For the high throughput screen (HTS) experiment, microplates were preloaded with medium and then processed by a Seiko pin-transfer robot with a Caliper Twister II robotic arm. The robot was programmed to add 2-3 nmol chemical library compound solubilized in 0.3 μl DMSO to each assay well. In additon to assay wells, every microplate included 16 positive and 16 negative control wells supplemented or not with 4 nmol chloramphenicol, respectively. 25 μl aliquots of the starter culture kept on ice were added to both assay and control wells and microplates were incubated at 42° C. in humidified chambers (humidity>85%). After 20 hours, the OD₆₀₀ was measured using a plate reader in absorbance mode. The screen was carried out in duplicate. Compounds that reduced bacterial growth by 90% or more were defined as screen positives and retested once for validation using the HTS protocol.

### Growth Inhibition Assay

Bacterial cultures were grown at 37° C. to an OD₆₀₀ of 0.6 and diluted 100-fold with ice-cold medium. The growth medium was Brain Heart Infusion (BHI) broth for staphylococci, enterococci, and bacilli. Clostridium perfringes was grown in BHI supplemented with 0.5% yeast extract and 0.1% L-cysteine in a nitrogen atmosphere. E. coli BL21 was cultured in cation-adjusted Mueller-Hinton II medium supplemented with 0.005% Tween-80. Growth inhibition was carried out in triplicate using 96-well microplates. Assay plates were preloaded with 100-μl aliquots of two-fold dilution series of compound prepared in growth medium and 10 μl of ice-cold starter culture was added to every assay well. The plates were incubated at 37° C. for 18-22 hours and the density of cultures was recorded with a plate reader. Normalized OD₆₀₀ measurements were used to derive the minimum inhibitory concentration (MIC) for growth for each compound. Software package GraphPad Prism 5 was used to calculate the 50% growth inhibitory concentration (IC₅₀) values.

### Cytotoxicity Assay

Human promyelocytic leukemia HL-60 cells (American Type Culture Collection number CCL-240) were maintained in RPMI 1640(CellGro) supplemented with 10% heat-inactivated fetal bovine serum (HyClone), 2mM L-glutamine, penicillin (50 units×ml⁻¹) and streptomycin (50 μg×ml⁻¹). Cell cultures were grown at 37° C. in 5% CO₂ until cell density reached 10⁶ cell×ml⁻¹. Cells were washed three times and suspended in Dulbecco's modified eagle medium (DMEM, Invitrogen). Microtiter plates (96 wells) were loaded with 10⁴ cells per well suspended in 100 μl DMEM containing increasing concentrations of compound. The plates were incubated for 4 hours at 37° C. in 5% CO₂. The cytotoxicity of compounds was assessed by measuring the activity of lactate dehydrogenase (LDH) released from cells that were damaged during the incubation. The LDH assay was performed in triplicate using the Cytotoxicity Detection Kit according manufacturer's instructions (Roche). LDH measurements were normalized as percentage of the total LDH activity in a cell lysate.

### Inhibition of LTA Synthesis by Small Molecules

An overnight culture of S. aureus RN4220 was diluted 100-fold in BHI medium supplemented or not with a sub-inhibitory concentration of hit compound. The cultures were incubated at 37° C. and bacterial growth was monitored over time until the control culture without compound reached an OD₆₀₀ of 1.0. Aliquots of 1 ml were removed from each culture and mixed with 0.5 ml glass beads (0.1 mm diameter). Bacteria were lysed in a bead beater and glass beads were removed by centrifugation (1 min at 200×g). The supernatant was centrifuged again (10 min, 16,000×g) to sediment cell debris containing cell-associated LTA. The pellet was suspended with 0.5 M Tris-HCl (pH 8.0)/2% SDS buffer in a volume normalized according OD₆₀₀ values. Samples were heat-treated at 95° C. for 30 min and cleared by centrifugation. Supernatants were separated by SDS-PAGE and analyzed by immunoblotting using a monoclonal antibody to detect LTA and polyclonal antibodies for LtaS and SrtA. Immune-reactive signals for LTA and LtaS were normalized against the envelope protein SrtA that remains unaffected by LTA synthesis inhibitors.

### Electron Microscopy

Bacteria were grown on solid medium and washed in water prior to fixation with 2% glutaraldehyde in phosphate buffered saline (PBS). Sample processing was performed as described earlier (Garufi et al. 2012). Examination of specimen was performed with a Fei Nova NanoSEM 200 scanning electron microscope (FEI Co., Hillsboro, Oreg., USA). The SEM was operated with an acceleration voltage of 5 kV and samples were viewed at a distance of 5 mm. Thin sectioning of samples was performed as described (Garufi et al. 2012) and images were recorded using a Tecnai F30 (Philips/FEI) transmission electron microscope (Field emission gun operating with a 300-kV accelerating voltage, using a magnification of 15,000 to 30,000×) and a high performance CCD camera with a 4 k×4 k resolution. Images were captured using Gatan DigitalMicrograph software and processed using Adobe Photoshop (Adobe, San Jose, Calif., USA). The thickness of the cell wall envelope was determined by examining at least 15 properly thin sectioned cells observed on micrographs obtained by transmission electron microscopy and as described earlier (Garufi et al. 2012). Data were plotted in Graphpad Prism 5.0 and the Student's t test (unpaired, 2-tailed) was used for statistical analyses.

### Light Microscopy

Cells were fixed using 4% buffered formalin and observed. Images were obtained with a CCD Camera on a Olympus IX81 microscope using 100× or 40× objectives. The lengths of bacilli was measured directly from acquired DIC images using ImageJ, and converted to lengths in microns using reference images with an objective micrometer. The data were displayed in a box and whisker plot. The Student's t test (unpaired, 2-tailed) was used for statistical analyses.

### Biochemical Assays

Recombinant eLtaS and SrtA were used to examine protein interactions with a synthetic phosphatidylglycerol modified with nitro-benzoxadiazole (NBD-PG). For this study, we used 1-palmitoyl-2-{12-[7-nitro-2-1,3-benzoxadiazole-4-yl)amino]dodecanoyl]-sn-glycerol-3-[phospho-rac-(1-glycerol) purchased from Avanti Polar Lipids. eLtaS was cloned using vector pProEX (In vitrogen, Life Technologies) as described by Lu et al. (Lu et al. 2009). SrtA was purified using plasmid pHTT27 as described by Ton-That et al. (Ton-That et al. 2002). Both proteins were purified over nickel-nitrilotriacetic acid resin as described (Ton-That et al. 2002). To study the interaction with NBD-PG, size-exclusion HPLC was performed with a BioBasic SEC300 column equilibrated in a 50 mM HEPES-KOH buffer, pH 7.5 containing 10 μM MnCl₂. Chromatograms were recorded by measuring absorbance at 460 nm to detect the presence of NBD-PG.

### Animal Experiments

Inhibitor solution was prepared freshly prior to every injection. Briefly, a frozen stock of compound 1771 (1) was pre-warmed at 37° C. for 5 minutes and suspended into Compound buffer (20 mM HEPES-KOH, pH 7.5, 100 mM NaCl). The clinical isolate Newman was used for infection since it has been extensively characterized in our laboratory to study the therapeutic effects of small molecule inhibitors (McAdow 2011). In vitro, strain Newman displayed similar sensitivity toward compound 1771 as strain USA300. An overnight culture of strain Newman was diluted 1:100 into fresh TSB and grown until an OD₆₀₀ of 0.4. Bacteria were centrifuged at 7,000×g, washed, and suspended in one-tenth volume of PBS. Six week-old female BALB/c mice (n=15) (Charles River) were injected retro-orbitally with 1×10⁸ colony forming unit (CFU) suspension in 100 μl of PBS. Mice were monitored for survival over 10 days. Animals received either two injections of inhibitor (32 mg/kg) or Compound buffer (Mock) prior to infection and an additional six doses post-infection. Animal experiments were performed in accordance with the institutional guidelines following experimental protocol reviewed and approved by the Institutional Biosafety Committee (IBC) and the Institutional Animal Care and Use Committee (IACUC) at the Univ. of Chicago. Log-rank was performed to analyze the statistical significance by Prism (GraphPad Software, Inc.) and P values less than 0.05 were deemed significant.

### Example 2

### High Throughput and Primary Screens for Growth Inhibitors of S. Aureus

S. aureus lacking the ltaS gene is unable to grow at temperatures above 37° C. (Oku et al, 2009). This phenotype was utilized to screen for candidate compounds that inhibit growth of wild type S. aureus strain RN4220 at 37° C. A growth assay amenable to HTS using 384-well microplates was developed. A single colony of S. aureus RN4220 was inoculated in cation-adjusted Hinton Mueller II broth supplemented with 0.005% Tween-80 and incubated overnight. Next day, the culture was diluted in fresh medium and incubation was continued until the optical density reached a bacterial titer equivalent to 5×10⁸ colony forming units (CFU) per ml. Culture aliquots (containing approximately 3.125×10⁴ CFU) were dispensed in 384-well plates containing small molecule compounds. The inhibitory activity of candidate compounds was compared to that of chloramphenicol (80 μM) or medium alone (no antibiotic; no compound). Incubation of cultures with chloramphenicol (80 μM, positive control) fully abolished growth of S. aureus strain RN4220 (100% inhibition as compared to medium alone, negative control). A screen hit was defined as a compound that reduced growth by 90% or more as compared to untreated cultures (negative control). The quality of the screen was assessed for every assay plate with the Z′-factor, a measure for data variability within the dynamic range of the assay (Zhang et al. 1999). Small molecule libraries from different vendors (Asinex, ChemBridge, ChemDiv, Enamine, LifeChemicals, Maybridge, TimTec) were tested in duplicate at the National Screening Laboratory for Regional Centers of Excellence in Biodefense and Emerging Infectious Disease (NSRB) at Harvard Medical School (Boston, Mass.). The screen included a total of 167,405 compounds and yielded 595 screen positives corresponding to a primary hit rate of 0.36% (Table 1). Hit compounds were subjected to a validation experiment whereby growth inhibition was tested once more using the 384-well plate format. 308 compounds were confirmed as screen positives displaying growth inhibition greater than 90% (Table 1). A computational analysis (World Wide Web at Molinspiration.com) of molecular properties of the 308 hit compounds (i.e. molecular weight, polar surface area, number of hydrogen bond donors and acceptors, and bond rotation) was conducted to determine violations of Lipinski's rule-of-five. The NSRB informatics group performed an analysis of promiscuous inhibitory activity and the NSRB medical chemistry group provided an evaluation on suitability for drug development. The analysis identified 116 compounds with drug-like properties that were commercially available and purchased for secondary screening experiments.

TABLE 1
SUMMARY OF HIGH-THROUGHPUT SCREEN

| | | Compound |
|---|---|---|
| Screening step | Assay description | count |
| Primary screen | Compound library screen - total | 167,405 |
| | Screen hits - identified ¹⁾ | 595 |
| | Compound validation test - total | 595 |
| | Screen hits - confirmed ¹⁾ | 308 |
| Secondary screen | Dose-response analysis - total | 116 |
| | MRSA inhibitor - no E. coli inhibitor, | 43 |
| | not cytotoxic ²⁾ | |
| | MRSA inhibitor - no E. coli inhibitor, | 6 |
| | cytotoxic ²⁾ | |
| | MRSA inhibitor - E. coli inhibitor | 24 |
| | MRSA inhibitor - E. coli inhibitor | 2 |
| | and cytotoxic ²⁾ | |
| | Not active | 41 |

¹⁾ Growth inhibition was ≧90% using compound library stock solutions at a final concentration of approximately 40-60 μM;
²⁾ Cytotoxicity of small molecules was measured using HL60 cells.

### Example 3

### Identification of an Inhibitor of LTA Synthesis

The 116 compounds with strong inhibitory activity were subjected to a dose-response analysis and their inhibitory attribute and cytotoxicity were assessed for specificity. To this end, growth inhibitory dose-responses were compared between S. aureus strain USA300 (MRSA) and E. coli K12 (Table 1). The overall cytotoxicity of compounds was assessed using human promyelocytic leukemia (HL-60) cells. This secondary screen revealed 43 possible candidates that specifically inhibited growth of MRSA without affecting the growth of E. coli or viability of HL-60 cells. The secondary screen identified 6 molecules that specifically inhibited growth of MRSA without affecting the growth of E coli but did affect viability of HL-60 cells (1592-E15, 1739-J18, 1775-G05, 1776-J05, 1849-L21 and 1586-O03). Sub-lethal concentrations of the 43 candidate compounds were used to examine the production of LTA in S. aureus using extracts of bacterial cultures grown with or without sub-inhibitory concentration of hit compounds. Three compounds, compound 1771, 1650-C01, and 1650-I01, were found to affect the production of LTA of S. aureus.

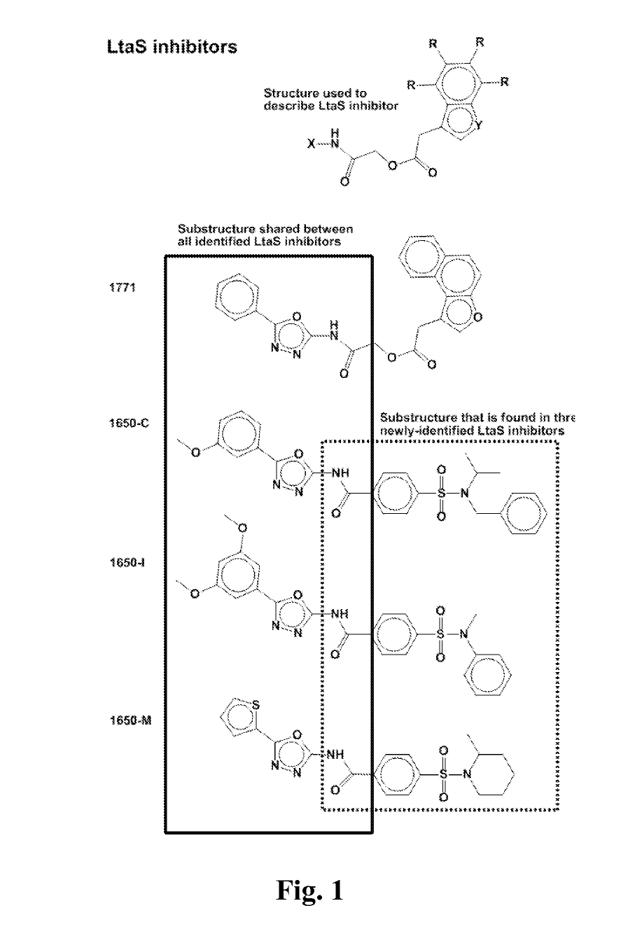

The formula at the top of FIG. 1 represents the generalized version of an LtaS inhibitor of the invention. Within the solid rectangle is a substructure that is shared between all four LtaS inhibitors. Within the dashed rectangle is a substructure common to the 1650 group of inhibitors.

### Growth Inhibitor 1771 Targets the LTA Biosynthesis Pathway

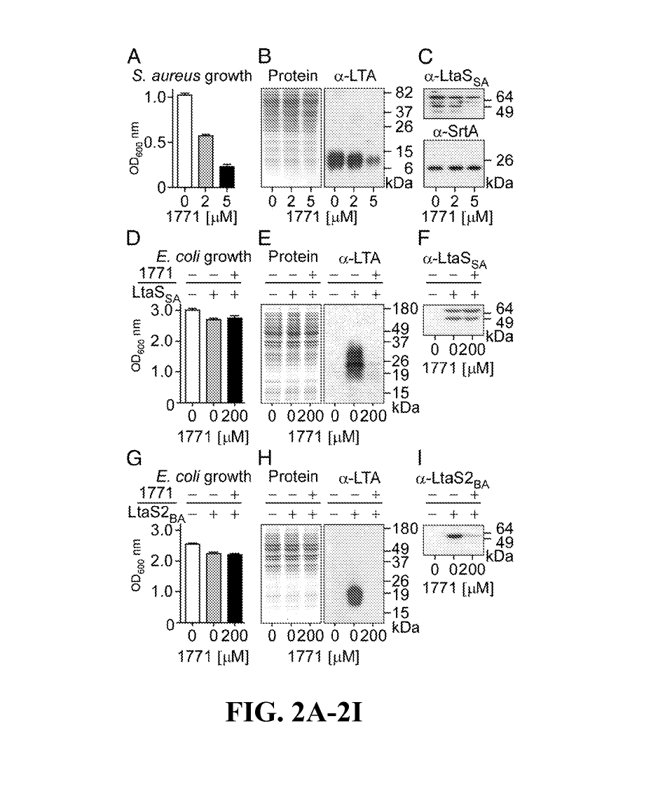

An overnight culture of S. aureus was diluted in the presence of increasing concentrations of compound 1771 dissolved in DMSO. For this experiment, all cultures received the same volume of DMSO containing 0, 2 or 5 μM of compound 1771 and were monitored until the OD₆₀₀ of the control culture (DMSO only) reached a value of 1.0 (FIG. 2A). Addition of 2 or 5 μM of compound 1771 reduced growth of staphylococci by 50 and 75%, respectively (FIG. 2A). Cultures were normalized to the same density and extracts were prepared and separated by SDS-PAGE for either visualization of proteins by Coomassie staining (FIG. 2B) or identification of immune reactive signals following transfer to nitrocellulose membranes (FIG. 2C). No noticeable difference could be observed between Coomassie stained samples suggesting that the compound did not affect overall protein synthesis and turn over (FIG. 2B). Western blot analyses confirmed that LTA production was reduced upon incubation of cultures with compound 1771 (FIG. 2C). Production of the membrane protein Sortase A (SrtA) that anchors secreted proteins to the cell wall remained unaffected. The LtaS enzyme appeared to be slightly unstable in the extract corresponding to incubation with 5 μM of compound 1771.

Production of the glycerolphosphate polymer can be recapitulated in E. coli by expression the gene ltaSSA on a plasmid (Grundling, 2007). The growth of E. coli expressing or not ltaSSA was not affected following incubation of cultures with and without 200 μM of compound 1771 (FIG. 2D) and in turn, extracts from these samples showed no difference in protein content when visualized by Coomassie staining of SDS-PAGE (FIG. 2E). Strikingly, the production of LTA as promoted by plasmid encoded LtaSSA was abrogated when the culture was incubated with 200 μM of compound 1771 (FIG. 2E; right panel) although plasmid encoded LtaSSA could be visualized by immunoblot in both extracts of cells carrying the plasmid (FIG. 2F). Production of LTA in E. coli can also be achieved by expressing the enzyme LtaS2BA of B. anthracis on a plasmid (Garufi et al. 2012). As with plasmid encoded LtaSSA, expression of LtaS2BA did not affect the growth of E. coli nor global protein synthesis (FIGS. 2G-2H). LTA production as visualized by western blot of extracts was only observed for culture samples expressing LtaS2BA and was completely abolished when cultures where further incubated with 200 μM of 1771 (FIG. 1H; right panel). Production of LtaS2BA was slightly affected by the presence of 200 μM of 1771 (FIG. 21). Together, the data suggest that compound 1771 inhibits LTA synthesis by altering the activity of LTA synthases.

### Example 5

### Compound 1771 Inhibits Growth of Many Gram-Positive and Antibiotic Resistant Pathogens

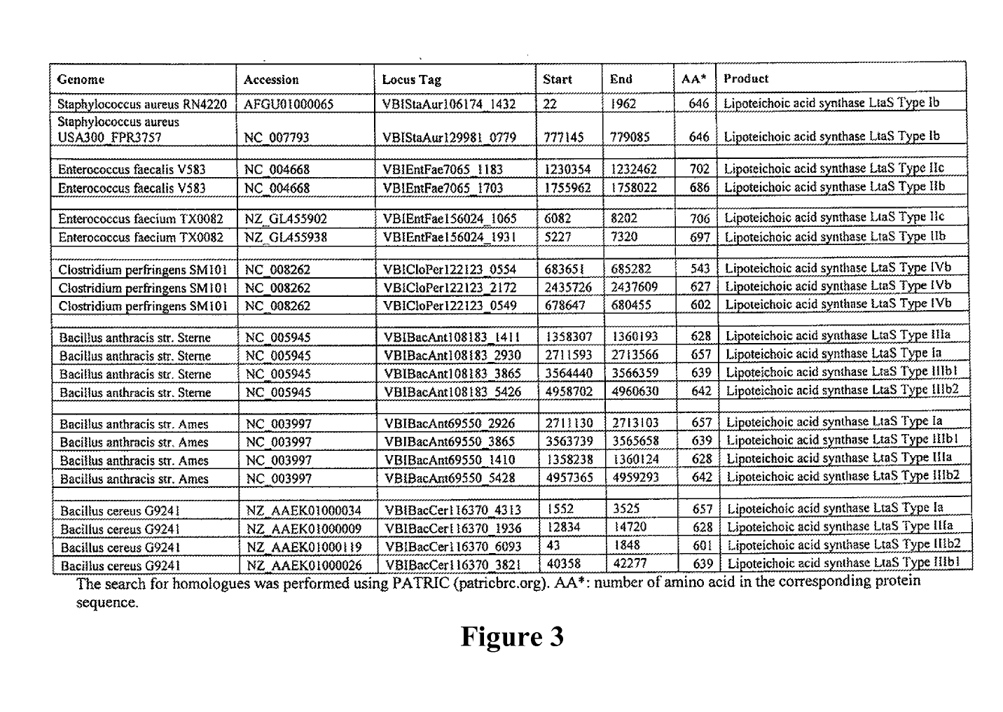

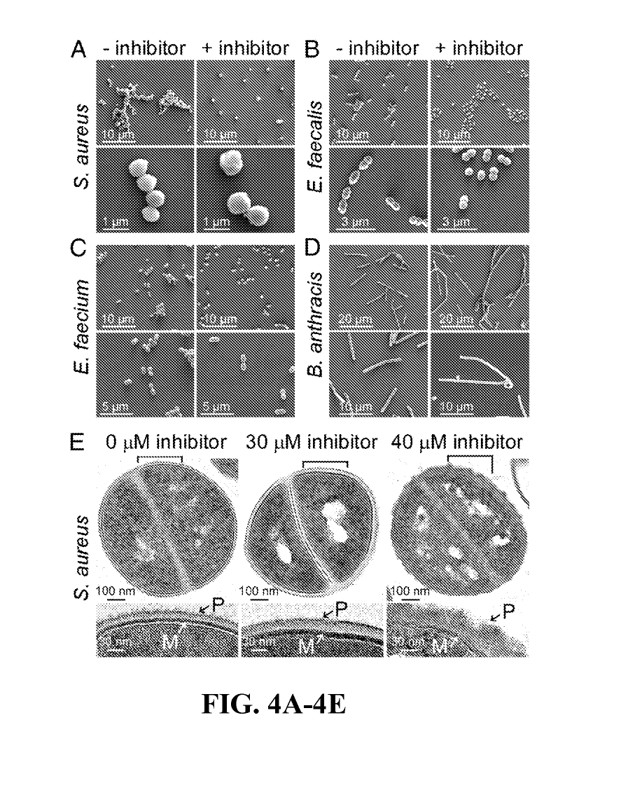

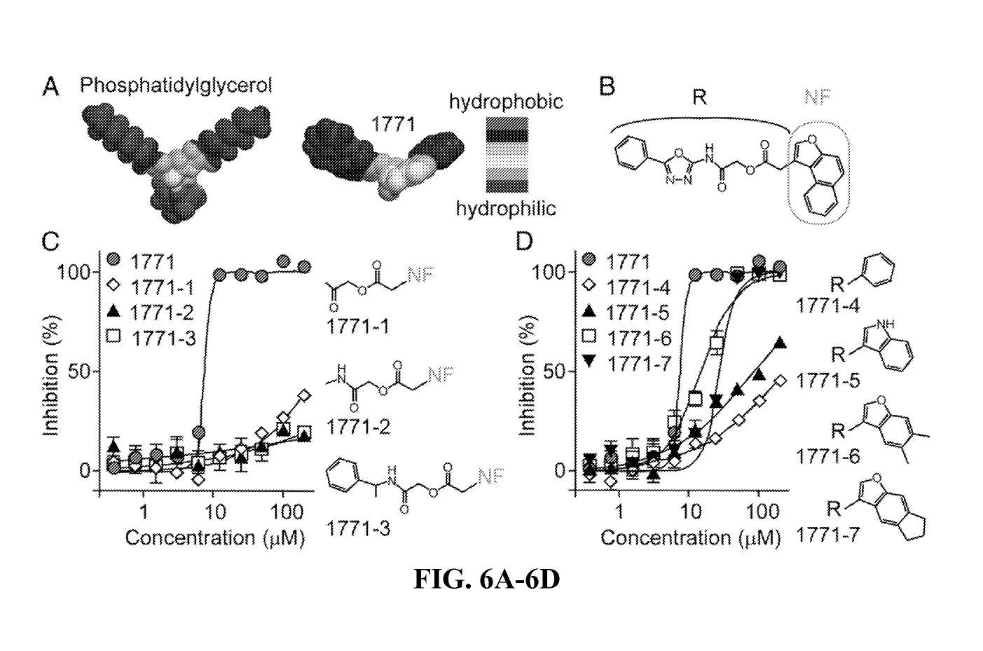

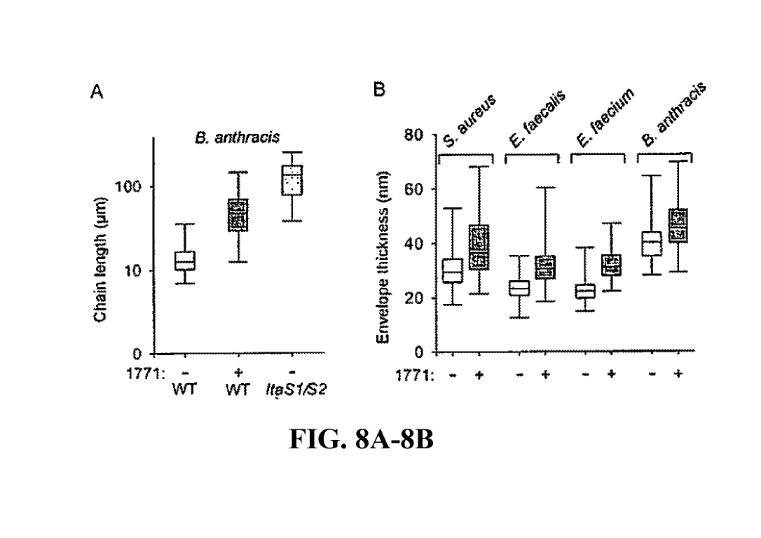

Whether candidate 1771 has broad-spectrum activity was investigated against several Gram-positive bacterial species that synthesize LTA. MRSA, vancomycin-resistant Enterococci (VRE), Clostridium perfringes, B. cereus and B. anthracis were selected for this study including Clostridium difficile for which an LtaS enzyme cannot be readily identified albeit that this organism also synthesizes an LTA-like molecule (Table 2). Staphylococcal strains encode one LtaS enzyme and we wondered whether antibiotic resistant strains such as MRSA USA300 might be as sensitive as the MSSA strain RN4220. A genomic analysis suggests that E. faecalis and E. faecium encode two LtaS enzymes that are related between enterococci (FIG. 3). While the function of these enzymes has not been examined, the existence and composition of enterococcal LTA polymers have been established (Greenberg, et al. 1996 and Theilacker et al. 2012). Production of LTA in C. perfringes has not been investigated but three LtaS homologues can be identified in the genome of this species (FIG. 3). Finally, we selected clinically relevant bacilli species B. anthracis strain Ames and B. cereus strain G9142. These organisms encode four LtaS homologues that are conserved between the two species (FIG. 3). The contribution of all four predicted LtaS enzymes has been validated in the attenuated B. anthracis strain Sterne (Garufi et al. 2012). Thus, a dose-response analysis for compound 1771 was performed with Gram-positive pathogens that encode between one and four LtaS enzymes (Table 2). C. perfringes with three predicted LtaS enzymes and bacilli with four LtaS enzymes were found to be the most susceptible to the inhibitory activity of compound 1771. Antibiotic resistant VRE and MRSA strains remained sensitive to compound 1771. C. difficile was also found to be sensitive to compound 1771 (Table 2). Scanning electron microscopy (SEM) was performed to visualize the effect of 1771 on bacterial growth. Specimen of staphylococci, enterococci and bacilli were examined following incubation of bacterial cultures with sub-lethal concentrations of compound 1771 (FIG. 4). Overall, cocci failed to form their typical clusters and in case of S. aureus, cells were physically separated following division (FIG. 4A-C). Larger magnifications of individual cells revealed deformation of the cell surface and cell shape and in most cases increased cell size (FIG. 4A-C). B. anthracis formed longer chains with noticeable curling at higher magnification suggesting aberrant cell separation (FIG. 4D). This morphology was reminiscent of that observed for the double ltaS1/ltaS2 mutant in strain Sterne (Garufi et al. 2012). In this mutant, bacterial chain length is increased and the ability to form colonies is reduced by 1,000 fold as compared to wild-type. Light microscopy was used to measure chain lengths of the isogenic ltaS1/ltaS2 mutant for comparison to wild type bacilli grown without or with a sub-lethal concentration of compound 1771 (FIG. 8). These measurements confirmed that incubation with compound 1771 leads to increased chain length albeit that this increase at sub-lethal concentration of compound is not as pronounced as that observed with the double mutant (FIG. 8). Aliquots of bacterial cultures were also prepared for thin-section transmission electron microscopy. This analysis suggested that the deformations of the cell surface observed by SEM could be attributed to a thickening and disorganization of the cell wall. Representative micrographs are shown for S. aureus RN4220 (FIG. 4E). Clearly, cells grown in the absence of inhibitor display a smooth surface and a well-organized envelope, a feature that is lost when cocci are grown with 40 μM inhibitor. Only, partial ruffling of the cell layers was observed when RN4220 was growth in the presence of 30 μM inhibitor (sub-lethal dose). Similar images were captured for thin-sectioned enterococci and bacilli (not shown) and used to measure envelope thickness. Box and whiskers plots of the data (FIG. 6B) shows a statistically increase in envelope thickness for all species grown in the presence of compound 1771. Thus, incubation with sub-inhibitory concentrations of compound 1771 alters the cell envelope of these bacteria and higher concentrations of compound lead to growth arrest. We surmise that these phenotypes are caused by inhibition of LTA synthesis catalyzed by one or more LtaS enzymes. Thus, we suggest that LTA is broadly required for viability of Gram-positive bacteria and LtaS enzymes can globally be inhibited by 1771.

TABLE 2
INHIBITORY ACTIVITY OF COMPOUND 1771
AGAINST GRAM-POSITIVE PATHOGENS

| | | | | |
|---|---|---|---|---|
| | | | | IC₅₀ (μM) |
| | | MIC | IC₅₀ | Confidence |
| Organism with | Strain | (μM) | (μM) ⁽¹⁾ | interval 95% ⁽¹⁾ |

One ltaS gene:

| | | | | |
|---|---|---|---|---|
| S. aureus - MRSA | USA300 | 50.0 | 14.0 | 12.6-15.7 |

Two ltaS genes:

| | | | | |
|---|---|---|---|---|
| E. faecalis - VRE | V583 | 50.0 | 27.3 | 24.4-30.4 |
| E. faecium - VRE | TX0016 | 50.0 | 23.3 | 20.1-27.1 |

Three ltaS genes:

| | | | | |
|---|---|---|---|---|
| C. perfringens | SM101 | 6.2 | 2.5 | 2.0-3.1 |

Four ltaS genes:

| | | | | |
|---|---|---|---|---|
| B. anthracis | Ames | 12.5 | 7.6 | 6.8-8.5 |
| B. anthracis | Sterne | 12.5 | 7.7 | 5.1-11.6 |
| B. cereus | G9142 | 25.0 | 10.7 | not calculated ²⁾ |

LTA but no obviousltaS gene using genome gazing

| | | | | |
|---|---|---|---|---|
| C. difficile | CD196 | 6.5 | 4.3 | 3.7-4.6 |
| C. difficile | JIR8094 | 12.5 | 8.2 | 6.7-10.0 |
| C. difficile | R20291 | 6.2 | 5.3 | not calculated |
| C. difficile | UK1 | 6.2 | 3.3 | 3.1-3.6 |

⁽¹⁾ IC₅₀ values and 95%-confidence intervals were calculated by fitting data with variable slope sigmoidal dose-response function (GraphPad Prism 5);
²⁾ Hill slope value of dose-response graph is >15.

### Example 6

### Inhibitory Activity of Compound 1771

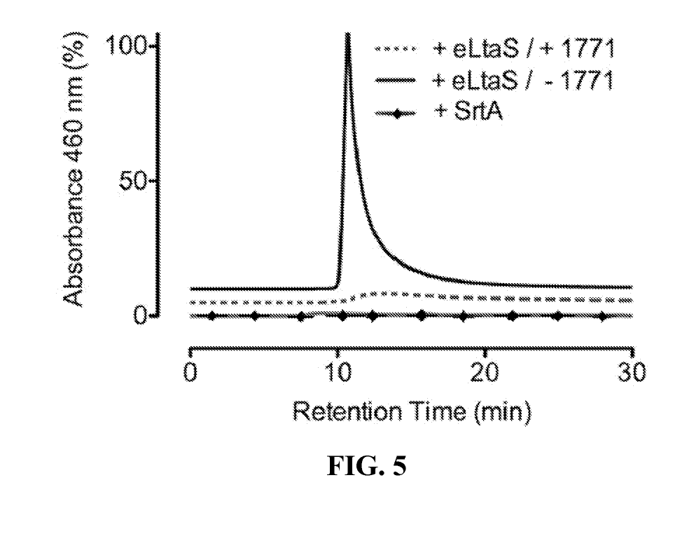

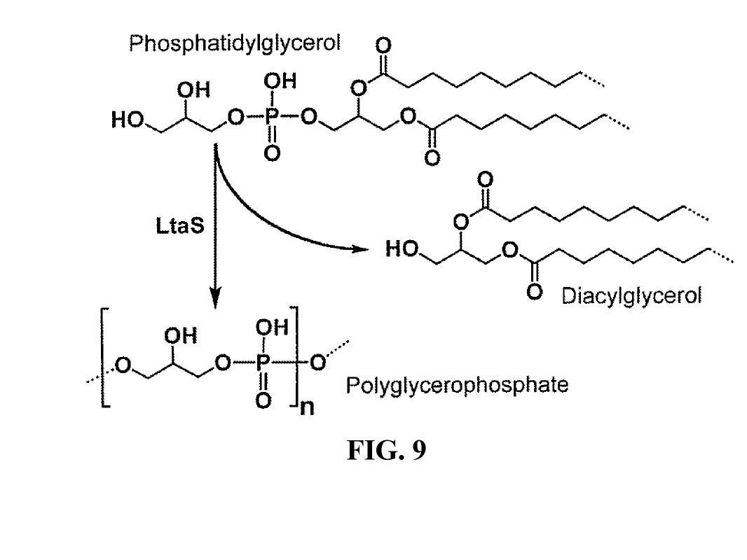

The mechanism of action of compound 1771 was explored in vitro using extracellular S. aureus LtaS (eLtaS) produced in E. coli (Grundling & Schneewind, PNAS 2007 and Garufi et al. 2012). eLtaS does not encompass the full length protein but rather a domain that is exposed on the cell surface of S. aureus (Grundling & Schneewind, PNAS 2007). X-ray crystallography confirmed the predicted function of LtaS by revealing that the structure of eLtaS solved at 1.2-Å resolution adopts a sulfatase-like fold (Lu et al. 2009). The presence of glycerol-phosphate in the crystal and supporting site-directed mutagenesis experiments confirmed that an active site threonine functions as nucleophile for phosphatidylglycerol hydrolysis and formation of a covalent threonine-glycerolphosphate intermediate (Lu et al. 2009). When phosphatidylglycerol is offered to purified eLtaS, it is effectively cleaved into hydrophilic glycerol and hydrophobic phosphatide (Karatsa-Dodgson et al. 2010). A similar clone was used to produce eLtaS and following incubation of phosphatidylglycerol, the expected cleavage products, glycerol and phosphatide, were obtained. However, formation of polyglycerolphosphate, the polymer synthesized in vivo, could not be found (data not shown; FIG. 9). Glycerol and phosphatide products were also observed when compound 1771 was added to the reaction although quantification proved difficult owing to the tedious separation of substrate and products in various solvent phases. Mass spectrometry measurements suggested that prolonged incubation of the compound with eLtaS did not result in adduct formation and the compound remained unmodified (no mass change). Next, we asked whether compound 1771 affects the interaction between eLtaS and phosphatidylglycerol. For this experiment, d size-exclusion HPLC was performed using a BioBasic SEC300 column (FIG. 5). The column was equilibrated in mobile phase buffer preloaded with phosphatidylglycerol (20 nmoles) modified with nitro-benzoxadiazole (NBD-PG) for photometric detection at 460 nm (Avanti Polar Lipids, Inc.). Injection of eLtaS (2 nmoles) but not Sortase A over the column triggered the elution of phosphatidylglycerol (FIG. 5; solid line and line designated with diamonds, respectively). To analyze the effect of the inhibitor on the enzyme-substrate interaction, the mobile phase was supplemented with 200 μM compound 1771 before injecting eLtaS. Co-elution of phosphatidylglycerol with eLtaS was reduced by more than 95% when the mobile phase was supplemented with 200 μM compound 1771 (FIG. 5; dashed line). As a control, injection of compound 1771 alone did not trigger the elution of phosphatidylglycerol. Structurally mimicry could account for the ability of compound 1771 to affect the interaction between phosphatidylglycerol and eLtaS. To investigate this possibility, we compared the distribution of hydrophobic and hydrophilic surface areas between compound 1771 and the phosphatidylglycerol molecule. Three-dimensional (3D) models showing the molecular hydrophobicity potential were generated using a web-based version of Galaxy 3D Structure Generator v2011.02 (Molinspiration: on the World Wide Web at www.molinspiration.com). Clearly, the 3D model of compound 1771 mimics the hydrophobicity pattern found in the central part of phosphatidylglycerol (FIG. 6A). To confirm this mimicry, we searched for compounds with related structures. The chemical designation of compound 1771 is 2-oxo-2-[(5-phenyl-1,3,4-oxadiazol-2-yl)amino]ethylnaphtho[2,1-b]furan-1-ylacetate and its structural formula can be arbitrary divided into the R and naphthofuranyl (NF) groups (FIG. 6B). We selected three derivatives with an intact NF group and different R groups (FIG. 6C; 1771-1/-3). When compared to intact compound 1771, none of these derivatives inhibited S. aureus growth (FIG. 4C; Table 3). Four compounds with intact R group and different NF groups were also identified (FIG. 6D; 1771-4/-7). These derivatives yielded increasing inhibitory activity upon acquisition of either naphtho or furan rings (FIG. 6D; Table 3). The relative loss of inhibitory activity by all seven substructures was similar between S. aureus strain RN4220 and B. anthracis strain Sterne (Table 3). Together, these findings suggest that compound 1771 is structurally related to phosphatidylglycerol and it disrupts the interaction between phosphatidylglycerol and eLtaS. It is unclear whether compound 1771 interacts directly with the enzyme or whether with the substrate. This latter mechanism would be reminiscent of vancomycin interaction with its peptidoglycan substrate preventing catalysis by transpeptidases.

TABLE 3
INHIBITORY ACTIVITY OF COMPOUND 1771 AND DERIVATIVES.

| | S. aureus RN4220 | | | B. anthracis Sterne | | |
|---|---|---|---|---|---|---|
| | | | IC₅₀ (μM) | | | IC₅₀ (μM) |
| Compound | MIC | IC₅₀ | Confidence | MIC | IC₅₀ | Confidence |
| Name | (μM) | (μM) | interval 95% | (μM) | (μM) | interval 95% |
| 1771 | 12.5 | 7.48 | 6.40-8.74 | 12.5 | 6.53 | 6.37-6.70 |
| 1771-1 | >400 | >400 | NA | >400 | 169.2 | 112.9-253.8 |
| 1771-2 | >400 | >400 | NA | >400 | >400 | NA |
| 1771-3 | >400 | >400 | NA | >400 | >400 | NA |
| 1771-4 | >400 | 242.4 | 165.7-354.7 | >400 | 223.6 | 142.9-349.8 |
| 1771-5 | >400 | 85.6 | 66.4-110.3 | 200.0 | 88.1 | 75.3-03.0 |
| 1771-6 | 50.0 | 15.2 | 12.6-18.3 | 25.0 | 14.8 | 13.1-16.7 |
| 1771-7 | 50.0 | 27.7 | 25.2-30.5 | 50.0 | 24.1 | 20.1-29.0 |

IC₅₀ values and 95%-confidence intervals were calculated by fitting data with variable slope sigmoidal dose-response function (GraphPad Prism 5). Corresponding graphs are shown in FIG. 4.

### Example 7

### Compound 1771 Delays Time-To-Death in a Mouse Model of Bacteremia

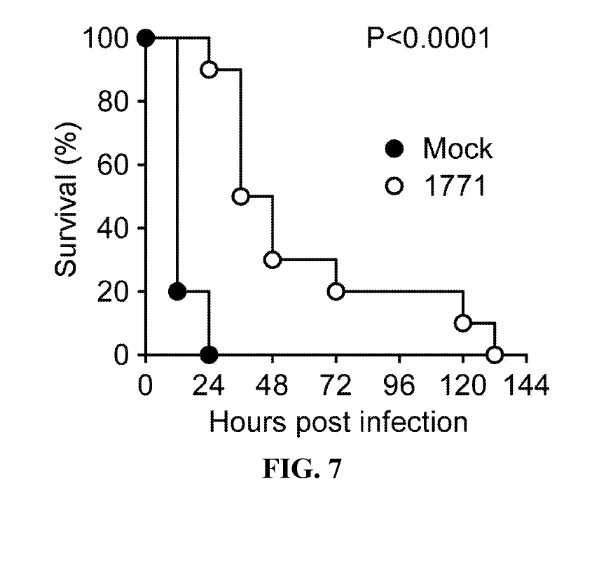

The therapeutic impact of compound 1771 during infection was examined. First, we evaluated the half-life of compound 1771 in vivo. Animals received intraperitoneal injections with 32 mg/kg of compound. The blood of three animals was drawn 1, 6 and 12 hours following intraperitoneal injections and the compound was extracted with methanol and chloroform, separated by reverse phase HPLC and subjected to mass spectrometry (data not shown). This analysis revealed two peaks corresponding to two fragments of compound 1771, none of which retained inhibitory activity. The conversion occurred between 3 and 6 hours. To evaluate, the efficacy of compound 1771, we used a mouse model of staphylococcal sepsis. Animals received intraperitoneal injections with 32 mg/kg of compound in 12-hour intervals. Treatment was initiated 24 hours prior to challenge and terminated at 72 hours post infection. Following challenge of mice via blood stream injection of 1×10⁸ CFU S. aureus Newman, mock treated animals died of sepsis within 24 hours (FIG. 7). In contrast, compound 1771-treated animals survived up to 132 hours, albeit that all animals in this cohort succumbed to the challenge (FIG. 7). Thus, although compound 1771 is unstable, administration of this compound in animals significantly delays the time to death following a lethal challenge with S. aureus in agreement with the notion that bacterial division is slowed down when LTA synthesis is inhibited.

### Example 8

### Further Validation of 1771

Compound 1771 Inhibits S. aureus Growth and LTA Synthesis

S. aureus variants that cannot express ltaS are unable to grow at 37° C. (Grundling 2007). We took advantage of the temperature-sensitive phenotype and screened compound libraries at the National Screening Laboratory for the Regional Centers of Excellence in Biodefense and Emerging Infectious Disease (NSRB) for candidate compounds that inhibit growth of S. aureus at 42° C. The primary screen identified 73 compounds with greater than 90% growth inhibition (SI, Supplementary Table 1). Thirty-one compounds were subjected to secondary screening, which included dose-response analyses for growth inhibition of MRSA as well as the Gram-negative microbe Escherichia coli. Fifteen compounds specifically inhibited the growth of MRSA but not of E. coli and displayed little or no cytotoxicity when added to HL-60 cells, a human promyelocytic leukemia cell (Table 1). One of these molecules, compound 1771, was identified as an inhibitor of LTA synthesis in S. aureus as follows.

Overnight cultures of S. aureus were diluted and incubated with increasing concentrations of compound 1771 (0, 2 or 5 μM), resulting in increased reduction of growth (FIG. 2A). Extracts prepared from staphylococcal cultures normalized to the same density were analyzed by SDS-PAGE. Coomassie-Blue staining of proteins suggested that incubation of staphylococci with compound 1771 did not alter the concentration of bacterial polypeptides (FIG. 2B). However, immunoblotting with 1,3-polyglycerol phosphate-specific monoclonal antibody (α-LTA) revealed that compound 1771 reduced the abundance of LTA (FIG. 2B). At higher concentration of compound 1771 (5 μM), LtaS-specific immunoreactive signals were reduced (FIG. 2C). As a control, the abundance of sortase A (SrtA), an enzyme that links proteins to peptidoglycan, was not affected in staphylococcal cultures treated with compound 1771 (FIG. 2C).

Expression of LtaS from S. aureus (ltaSSA) or Bacillus anthracis (ltaS2BA) in E. coli leads to the production of 1,3-polyglycerol phosphate, as LTA synthase can utilize phosphatidylglycerol (PG) substrate even from the membrane of Gram-negative bacteria (FIG. 2 E-H). Unlike its antibiotic activity in S. aureus, compound 1771 did not affect the growth of E. coli even at very high concentrations (200 μM) (FIG. 2D-G). Strikingly, E. coli synthesis of polyglycerol-phosphate via LtaS_{AS} (FIG. 2E) or LtaS2_{BA} (FIG. 2H) was abrogated in the presence of compound 1771. Of note, E. coli expression of LtaS2_{BA}, but not of LtaS_{AS}, was reduced in the presence of compound 1771 (FIG. 2F-I).

### Compound 1771 Inhibits the Growth of Gram-Positive Bacteria

To examine the spectrum of antibiotic activity for compound 1771, we analyzed Gram-positive bacteria harboring polyglycerol-phosphate LTA as well as LtaS homologues (FIG. 2). Compound 1771 inhibited the growth of antibiotic-resistant MRSA, for example the epidemic community-acquired isolate USA300 LAC, and VRE, i.e. vancomycin-resistant Enterococcus faeaclis and Enterococcus faecium whose genomes harbor two ltaS homologues (Table 3). Gram-positive bacteria with three (Clostridium perfringes) or four ltaS homologues (B. cereus and B. anthracis) appeared to be more susceptible to compound 1771-mediated growth inhibition than microbes with only one or two ltaS genes (Table 3).

Bacterial cultures incubated with or without sublethal concentrations of compound 1771 were examined by scanning electron microscopy (SEM) (FIG. 4). Treatment with compound 1771 dispersed cluster formation in S. aureus or chain formation in E. faecalis and E. faecium (FIG. 4A-C and Table 6). At higher magnification of SEM images, compound 1771-treated staphylococci and enterococci revealed an increase in cell size as well as deformations of their cell surface and shape (FIG. 4A-C). In the presence of compound 1771, B. anthracis formed longer chains of vegetative cells and displayed undulating deformations of its cylindrical cell shape (FIG. 4D and FIG. 8). Compound 1771-induced morphological changes resemble those observed for S. aureus and B. anthracis mutants with defects in LTA synthesis (trundling 2007; Garufi 2012). For example, the chain length of the B. anthracis ltaS1/ltaS2 mutant is increased as compared to wild-type (FIG. 8A) and its ability to form colonies is reduced by 1,000 fold (Garufi 2012).

Thin-section transmission electron microscopy (TEM) of S. aureus treated with compound 1771 revealed the thickening and structural disorganization of the cell wall envelope (FIG. 4E). The smooth surface and structural organization of the envelope were perturbed when staphylococci were grown in the presence of 40 μM compound 1771. Similar results were obtained with TEM images of thin-sectioned enterococci and bacilli, which prompted measurements of envelope thickness. The data revealed increases in envelope diameter for S. aureus, E. faecalis, E. faecium and B. anthracis grown in the presence of compound 1771 (FIG. 8B).

### Mechanism of LTA Synthesis Inhibition for Compound 1771

The three dimensional structure of the extracellular catalytic domain of LtaS has been determined (Schirner 2009, Lu 2009). Overall, eLtaS assumes a sulfatase-like fold, however its active site is distinct from that of sulfatases (Lu 2009). Threonine (T300) of LtaS together with residues E255, D475 and H476 coordinate a manganese ion and assemble to form a binding pocket for glycerol-phosphate (Lu 2009). As revealed from the co-crystal structure of eLtaS with glycerol-phosphate, one oxygen atom of the phosphate group is coordinated with Mg2+, whereas the remainder of the phosphate group is stabilized by hydrogen bonding with H416 and W354. Hydroxyl side chains of glycerol-phosphate form hydrogen bonds with H347, D349 and R356 (Lu 2009). Catalysis has been proposed to involve PG docking in the active site of eLtaS to enable nucleophilic attack from the deprotonated hydroxyl of T300, generating a glycerol-phosphate-threonine intermediate and releasing diacylglycerol. The glycerol-phosphate-threonine intermediate may subsequently be resolved by the nucleophilic attack from the terminal OH group of another PG, thereby extending the LTA chain by one glycerol-phosphate moiety (Lu 2009).

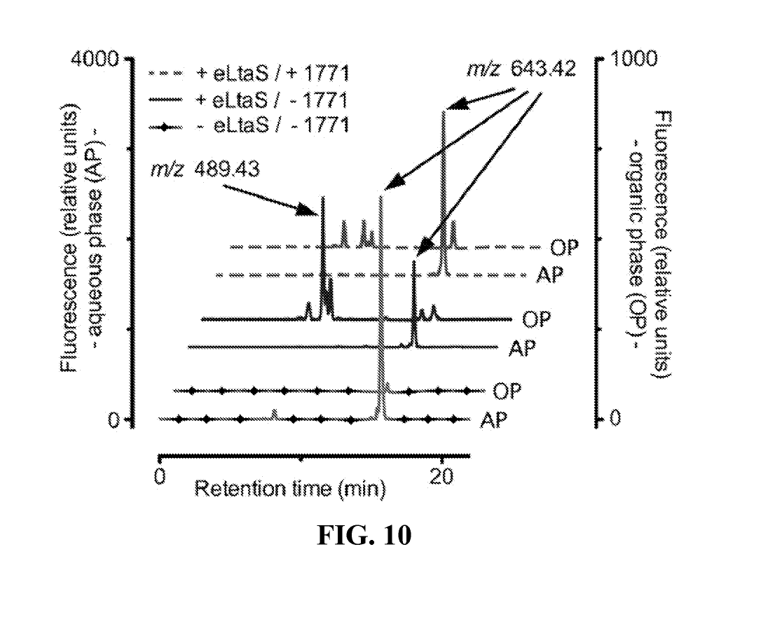

Incubation of purified eLtaS with compound 1771 followed by mass spectrometry analysis of enzyme and inhibitor did not reveal the formation of an eLtaS adduct or the cleavage of compound 1771. We used size exclusion HPLC with BioBasic SEC300 column equilibrated with 20 nmol nitro-benzoxadiazole glycerol-phosphate (NBD-GP) to measure binding of enzyme to substrate. Chromatography of 2 nmol eLtaS on BioBasic SEC300 column led to the elution of enzyme.NBD-GP complex and absorbance at 460 nm (FIG. 5). As a control, chromatography of 2 nmol sortase A, a transpeptidase that anchors surface proteins to peptidoglycan and does not bind to glycerol-phosphate (Ton-That 1999), did not elute NBD-GP from pre-equilibrated BioBasic SEC300 column (FIG. 5). When the mobile HPLC phase was supplemented with 200 μM LTA synthesis compound 1771, the ability of eLtaS to elute NBD-GP complexes from the pre-equilibrated column was abolished (FIG. 5). eLtaS-mediated cleavage of nitrobenzoxadiazole-PG (NBD-PGC6, with six carbon acyl chains) was used to determine whether compound 1771 inhibits LTA synthesis in vitro (Karatsa-Dodgson 2010). In the presence of enzyme (+eLtaS), but not in its absence (−eLtaS), NBD-PGC6 (m/z 643.42) was cleaved to generate NBD-DAGC6 (nitrobenzoxadiazole-diacylglycerol, m/z 489.43), as revealed by HPLC and mass spectrometry of chloroform extracted samples, separating NBD-PGC6 substrate in the aqueous phase (AP) from NBD-DAGC6 product in the organic phase (OP) (FIG. 10). Addition of 100 μM compound 1771 inhibited eLtaS-mediated formation of NBD-DAGC6 product from NBD-PGC6, indicating that the molecule inhibited LTA synthesis in vitro (FIG. 10).

The results in FIG. 5 and FIG. 10 suggest that compound 1771 may bind to the active site of eLtaS and prevent its association with PG. If so, the distribution of hydrophobic and hydrophilic surface areas may be similar for compound 1771 and PG. This was examined by generating three-dimensional (3D) models with the Galaxy 3D Structure Generator v2011.02 (at molinspiration.com) (FIG. 6A). The chemical designation of compound 1771 is 2-oxo-2-[(5-phenyl-1,3,4-oxadiazol-2-yl)amino]ethylnaphtho[2,1-b]furan-1-ylacetate and its structural formula can be arbitrarily divided into the R and naphthofuranyl (NF) groups (FIG. 6B). The NF and R groups appear to mimic the polar chains of PG (FIG. 6A). To analyze this possibility, we selected three derivatives with an intact NF group and different R groups for structure-activity-relationships (FIG. 6C; 1771-1/-3). When compared to compound 1771, none of these derivatives inhibited S. aureus growth (FIG. 6C; Table 3). Four compounds with intact R group and different NF groups were also identified (FIG. 6D; 1771-4/-7). These derivatives yielded increasing inhibitory activity upon acquisition of either naphtho or furan rings (FIG. 6D; Table 3). The relative loss of inhibitory activity by all seven structural derivatives was similar for S. aureus and B. anthracis (Table 3). Together, these findings indicate that compound 1771 is structurally similar to PG and prevents the interaction between PG and eLtaS.

Compound 1771 Prolongs the Survival of Mice with S. aureus Sepsis

To examine the therapeutic value of compound 1771, we evaluated its half-life in mice. Animals received a single intraperitoneal injection of 32 mg/kg compound 1771. The blood of three animals was drawn 1, 6 and 12 hours following 1771 injection and serum samples were extracted with methanol and chloroform, separated by reversed-phase HPLC and subjected to mass spectrometry. This analysis revealed two absorption peaks corresponding to cleavage fragments of compound 1771, none of which retained inhibitory activity. Full conversion of compound 1771 into its two cleavage fragments occurred between 3 and 6 hours following injection. The enzyme(s) responsible for compound 1771 cleavage are not yet known and it is not yet clear whether compound 1771 can be modified to resist cleavage whilst retaining its antibiotic activity.

To evaluate the therapeutic efficacy of compound 1771, we used a mouse model of staphylococcal sepsis (McAdow 2011). Animals received intraperitoneal injections with 32 mg/kg of compound in 12-hour intervals. Treatment was initiated 24 hours prior to challenge and terminated at 72 hours post infection. Following challenge of mice via bloodstream injection of 1×108 CFU S. aureus Newman, mock treated animals died of sepsis within 24 hours (FIG. 7). In contrast, compound 1771-treated animals survived up to 132 hours, albeit that all animals in this cohort eventually succumbed to the challenge (FIG. 7). Thus, although compound 1771 is unstable in mice with rapid loss of activity, its administration into animals delays time-to-death following a lethal challenge with S. aureus.

Selecting for S. aureus Variants with Increased Resistance to Compound 1771

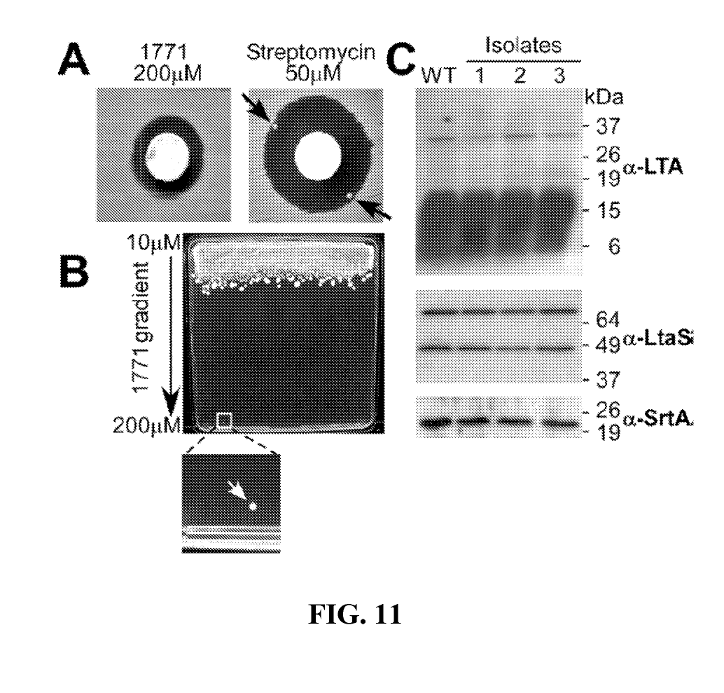

The therapeutic value of many antibiotics is limited because bacteria acquire resistance via spontaneous mutations that modify the drug target (Walsh 2000). For example, streptomycin blocks ribosomal protein synthesis (Anand 1960, Jones 1944), however mutations in rpsL, the structural gene for ribosomal protein S12, alter the polypeptide to prevent antibiotic access to the ribosome (Funatsu 1972). In contrast to streptomycin-resistant mutants, which arise at frequencies <10⁻⁷ (FIG. 11A), S. aureus RN4220 formed rare small colonies only after 3-4 days of incubation on agar media with a 10-200 μM gradient of compound 1771 (FIG. 11B). These observations suggest that resistant colonies cannot be isolated from S. aureus at frequencies ≦2×10⁻⁹. When analyzed for their resistance phenotype, each of the three independent colony isolates did not display significant changes in either the minimal inhibitory concentration (MIC) or the IC₅₀ values for compound 1771 (Table 7). Isolated strains produced LtaS and LTA with similar abundance as their S. aureus parent and did not harbor mutational alterations in the ltaS gene (FIG. 11C). Thus, S. aureus selection on agar plates did not lead to variants with a significant increase in resistance to compound 1771. This phenotype is similar to that reported for vancomycin (McCormick 1955-1956), a cell wall active antibiotic, which requires mutations in different genes for staphylococci to acquire an intermediary resistance phenotype (Walsh 1993, Yamakawa 2012).

### Example 9

### Growth Inhibitors 1650-C01, 1650-I01, and 1650-M01 Target the LTA Biosynthesis Pathway

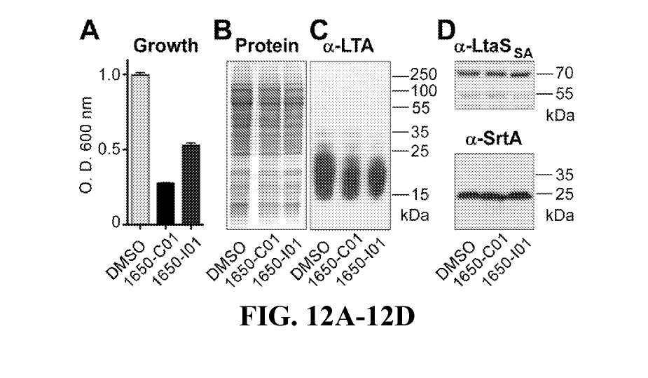

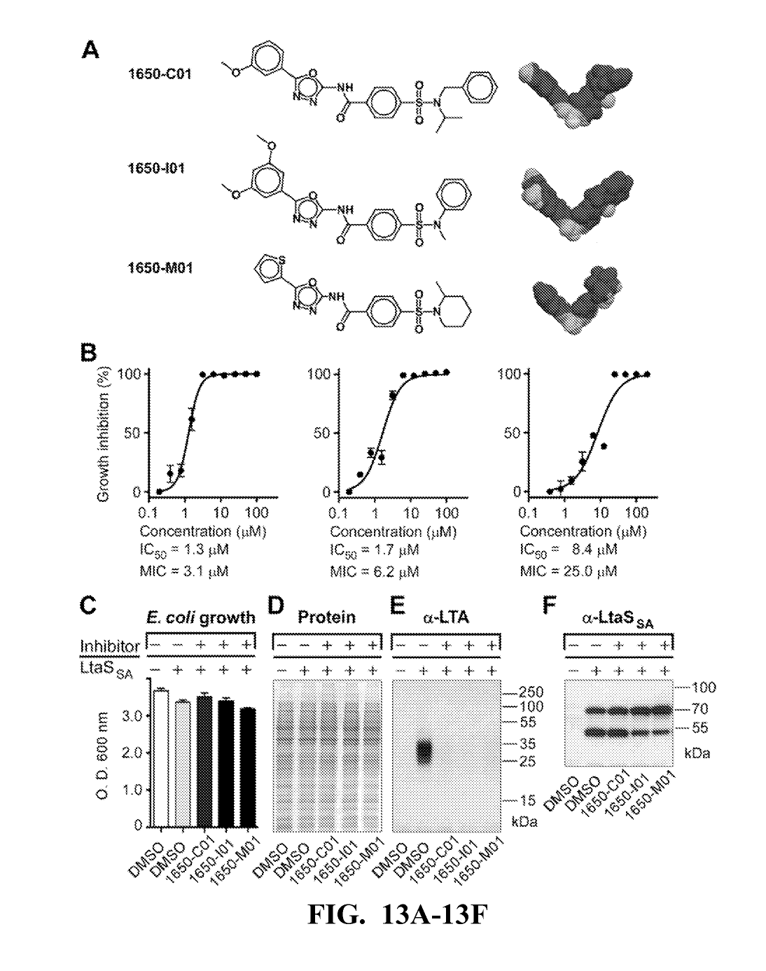

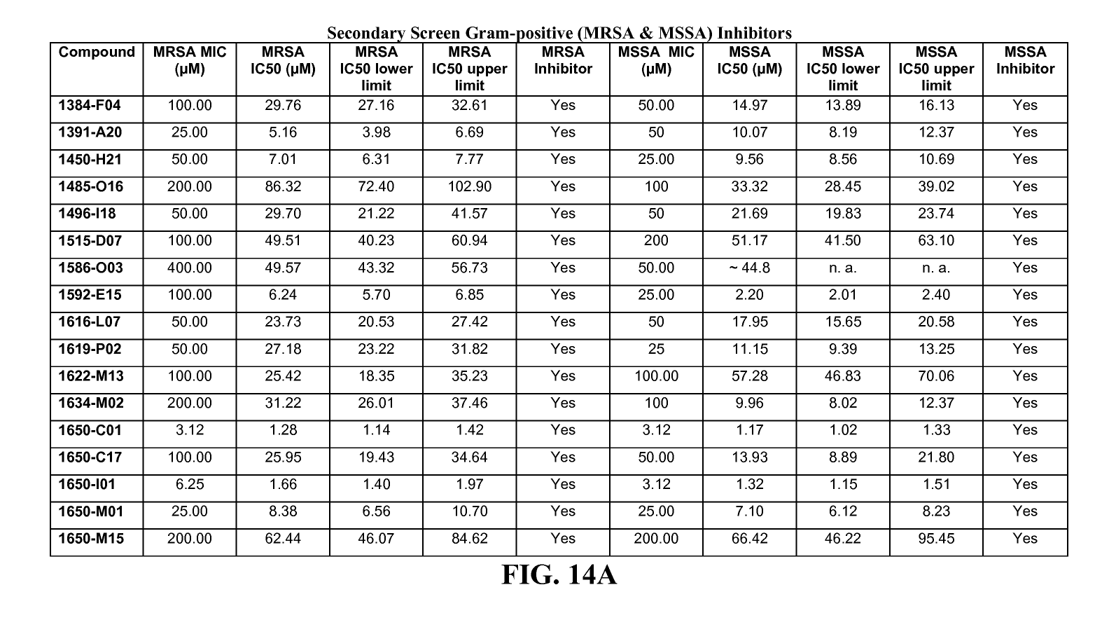

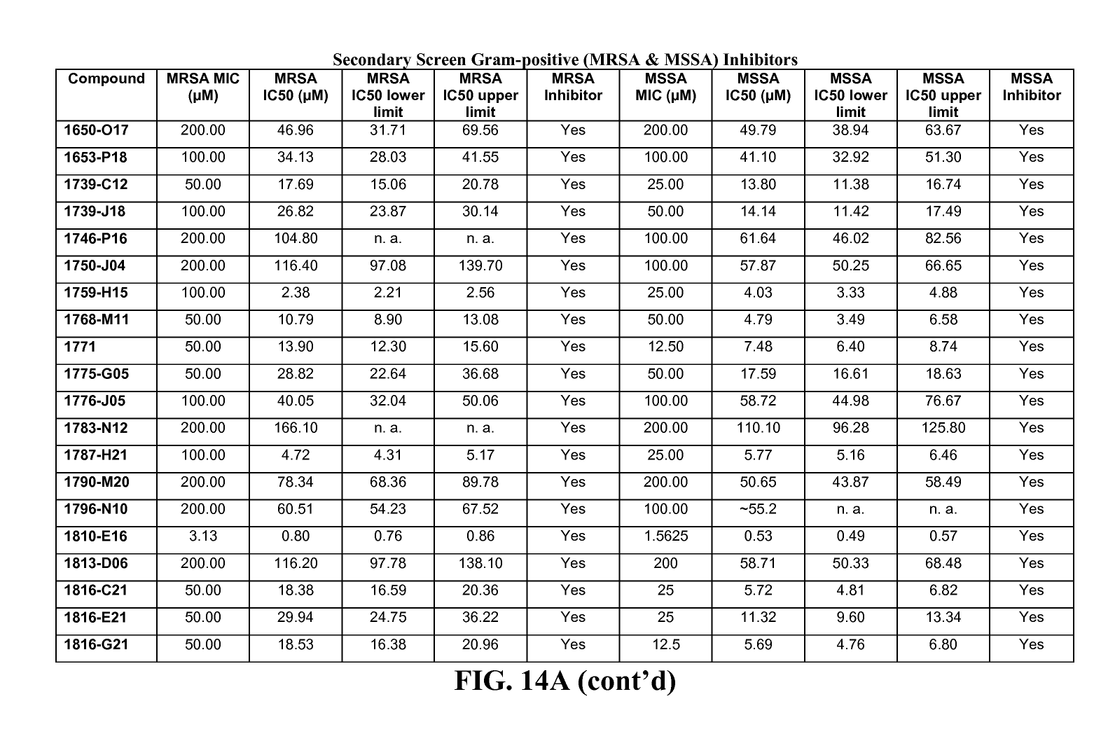

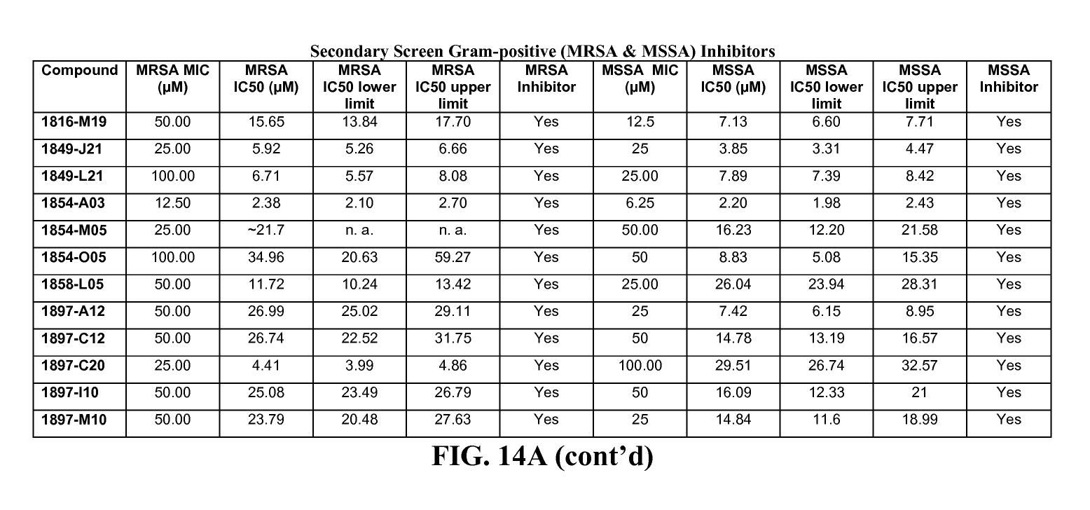

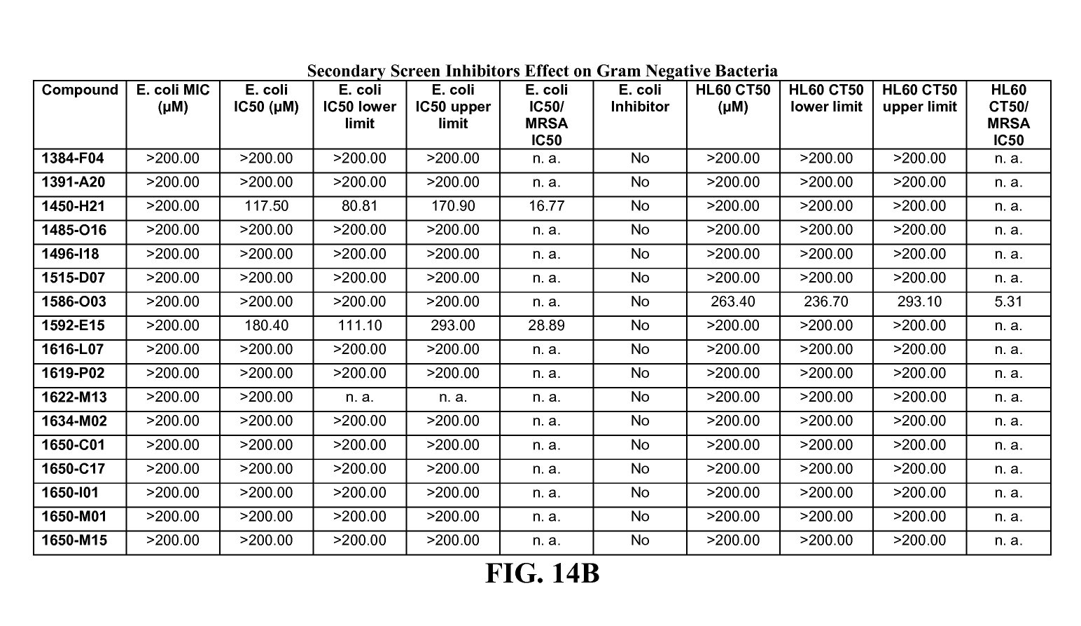

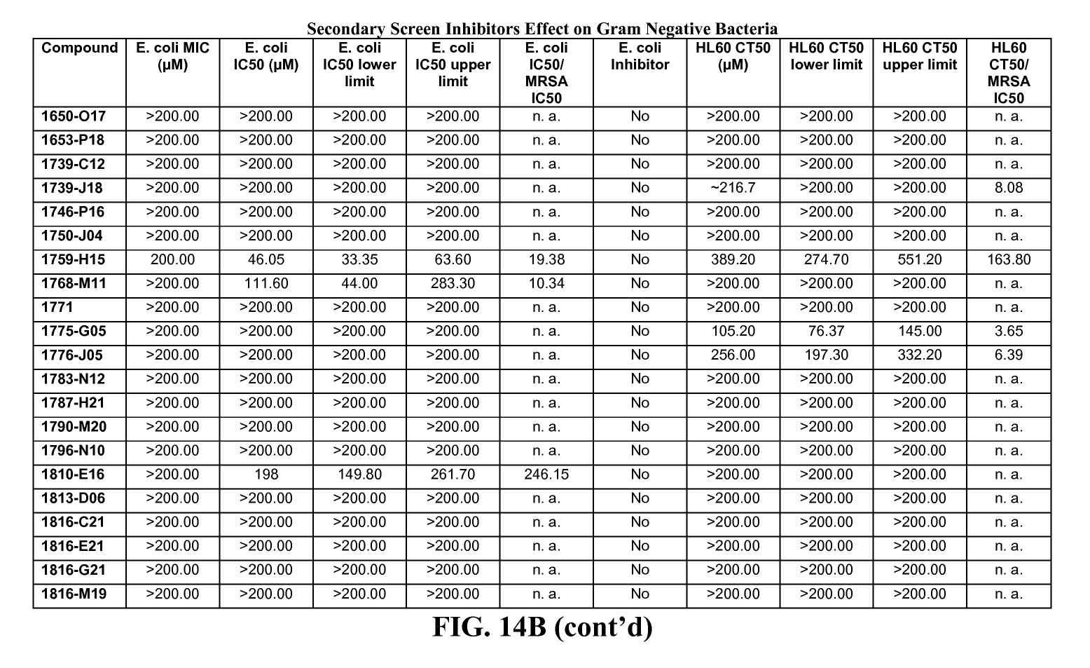

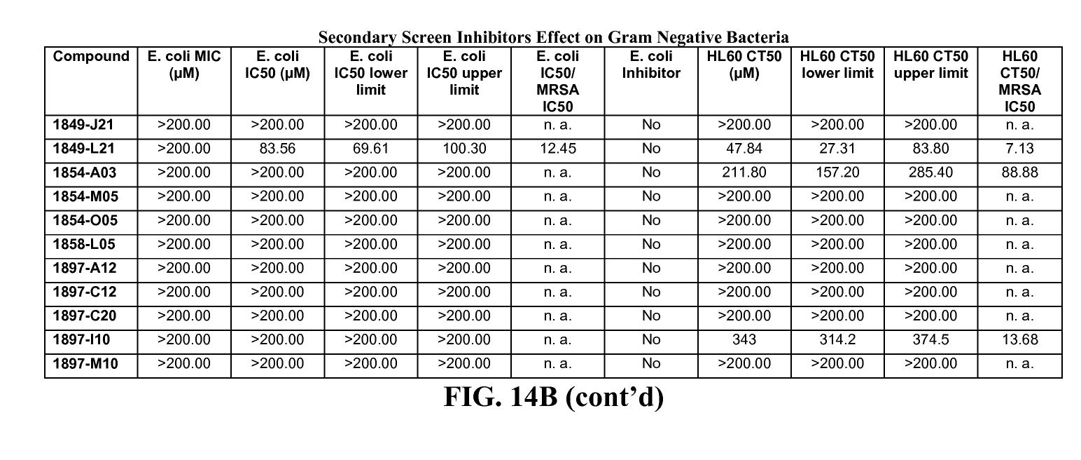

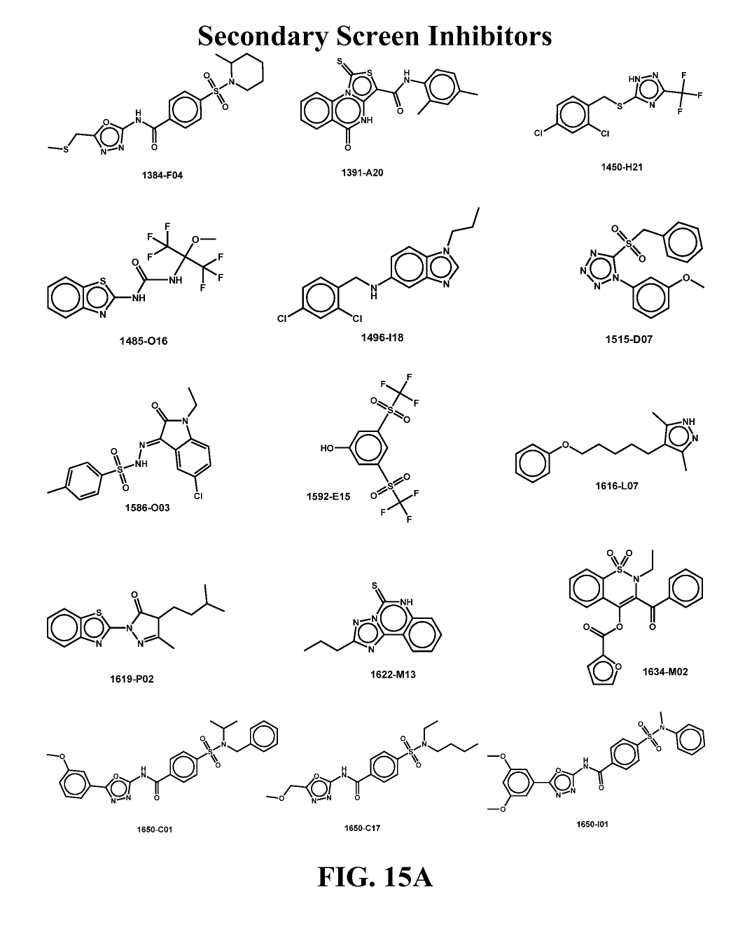

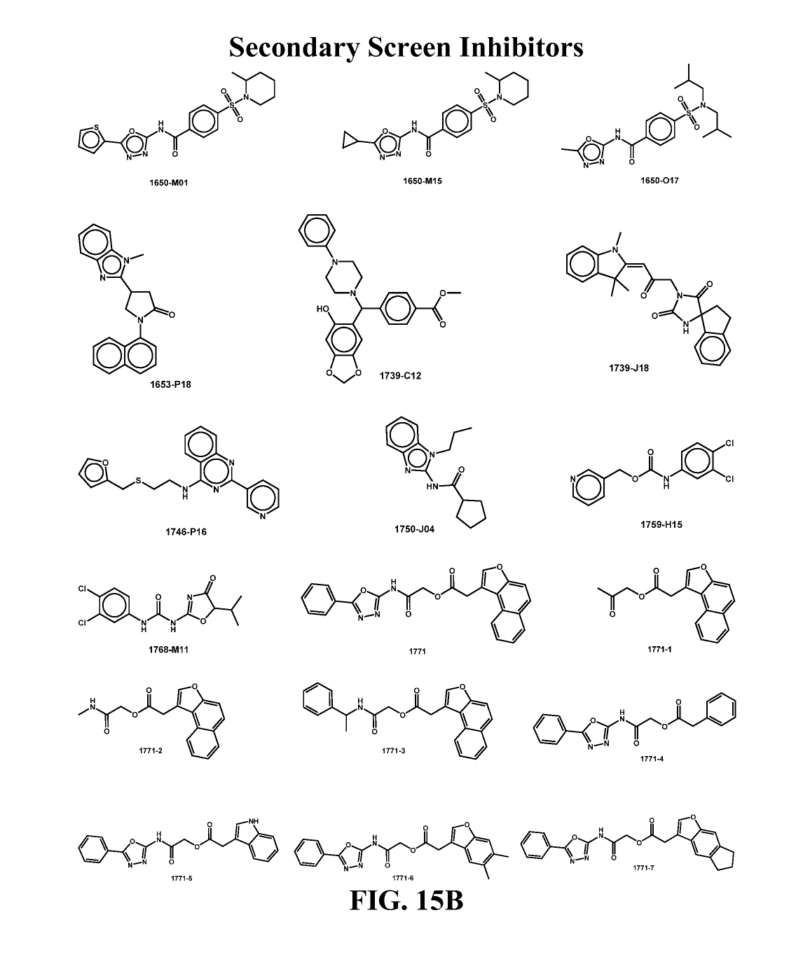

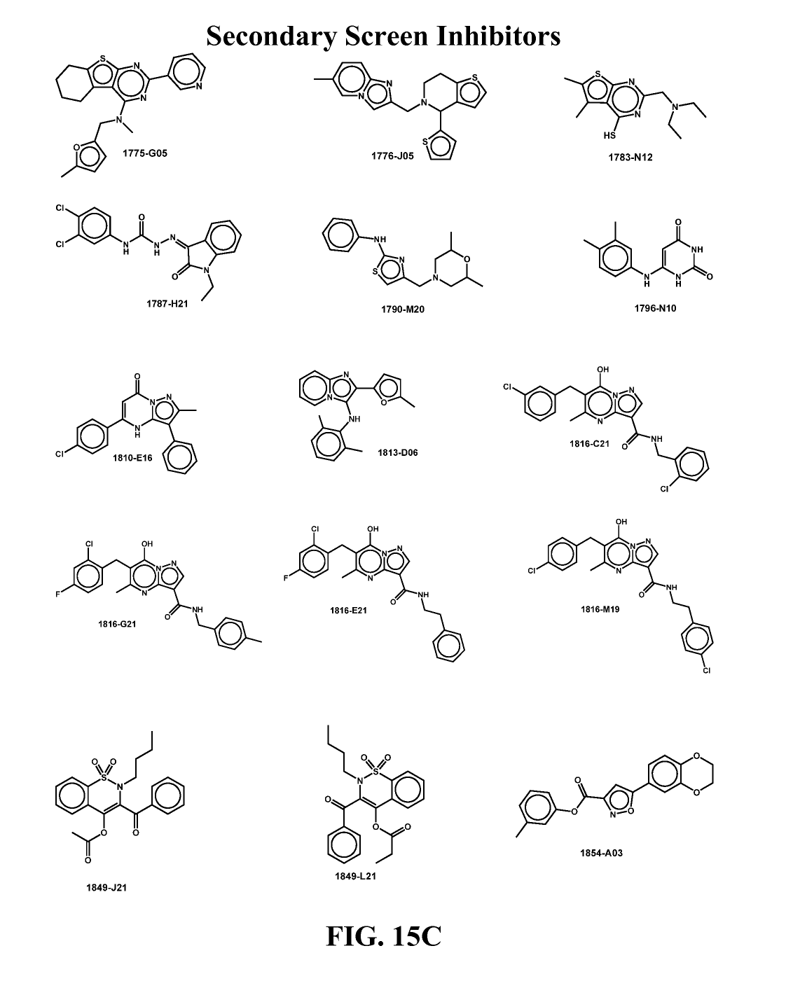

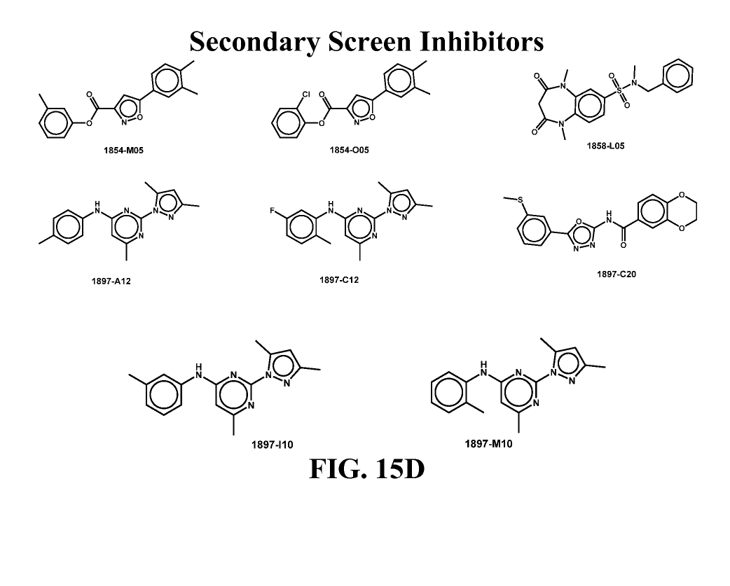

An overnight culture was diluted in medium supplemented with either 1% DMSO (control culture) or 1 μM compound 1650-C01 or 1650-I01. Bacterial growth was monitored until the OD600 of the control culture reached a value of 1.0 (FIG. 12A). Addition of 1 μM of compound 1650-C01 or 1650-I01 reduced growth of staphylococci by 75 and 50%, respectively (FIG. 12A). Cultures were normalized to the same density and extracts were prepared and separated by SDS-PAGE for either visualization of proteins by Coomassie staining (FIG. 12B) or identification of immune reactive signals following transfer to nitrocellulose membranes (FIGS. 12C and 12D). No noticeable difference could be observed between Coomassie stained samples suggesting that the compounds did not affect overall protein synthesis and turn over (FIG. 12B). Western blot analyses confirmed that LTA production was reduced upon incubation of cultures with compounds 1650-C01 and 1650-I01 (FIG. 12C). The abundance of LtaS was not affected by either inhibitor (FIG. 12D, upper panel) and the production of membrane protein Sortase A (SrtA) that anchors secreted proteins to the cell wall remained unchanged (FIG. 12D, lower panel).

A comparison of the structures of all 43 MRSA inhibitors identified one compound, 1650-M01, with similarity to 1650-C01 and 1650-I01 (FIG. 13A). Interestingly, the similarity extended to the 3D models derived from their primary structures. Like LtaS inhibitor 1771, the overall shape and the distribution of hydrophobic and hydrophilic surface areas partially resembles the predicted 3D structure of LtaS substrate phosphatidylglycerol (PG). The dose-response test with S. aureus USA300 verified 1650-C01, 1650-I01, and 1650-M01 as potent MRSA growth inhibitors with MIC values of 3.1 μM, 6.2 μM, and 25 μM, respectively (FIG. 13B).

Production of the glycerolphosphate polymer can be recapitulated in E. coli by expression of the ltaSSA gene on a plasmid. The growth of E. coli expressing or not ltaSSA was not affected following incubation of cultures with and without 200 μM of either compound (FIG. 13C) and in turn, extracts from these samples showed no difference in protein content when visualized by Coomassie staining of SDS-PAGE (FIG. 13D). Strikingly, the production of LTA as promoted by plasmid encoded LtaSSA was abrogated when the culture was incubated with 200 μM of 1650-C01, 1650-I01, or 1650-M01 (FIG. 13E) although plasmid encoded LtaSSA could be visualized by immunoblot in all extracts of cells carrying the plasmid (FIG. 13F). Together, the data suggest that compounds 1650-C01, 1650-I01, and 1650-M01 inhibit LTA biosynthesis by altering the activity of LTA synthases.

### Example 10

### Compounds 1650-C01, 1650-I01, AND 1650-M01 Inhibit Growth of Many Gram-Positive and Antibiotic Resistant Pathogens

Whether inhibitors 1650-C01, 1650-I01 and 1650-M01 have broad-spectrum activity was investigated against several Gram-positive bacterial species that synthesize LTA. MRSA, vancomycin-resistant Enterococci (VRE), and B. anthracis were selected for this study (Table 4). Staphylococcal strains encode one LtaS enzyme and we wondered whether antibiotic resistant strains such as MRSA USA300 might be as sensitive as the MSSA strain RN4220. A genomic analysis suggests that E. faecalis and E. faecium encode two LtaS enzymes that are related between enterococci (FIG. 3). While the function of these enzymes has not been examined, the existence and composition of enterococcal LTA polymers have been established. The B. anthracis genome encodes four LtaS homologues. The contribution of all four predicted LtaS enzymes has been validated in the attenuated B. anthracis strain Sterne. Thus, the dose-response analysis for compounds 1650-C01, 1650-I01 and 1650-M01 was performed with Gram-positive pathogens that encode between one and four LtaS enzymes (Table 4). S. aureus and B. anthracis were found to be the most susceptible to the LtaS inhibitors. The antibiotic resistant VRE strains remained sensitive to the 1650-type compounds.

TABLE 4
INHIBITORY ACTIVITY OF COMPOUNDS 1650-C01, 1650-I01, AND 1650-M01
AGAINST GRAM-POSITIVE PATHOGENS

| | | 1650-C01¹⁾ | | | 1650-I01¹⁾ | | | 1650-M01¹⁾ | | |
|---|---|---|---|---|---|---|---|---|---|---|
| | | | | IC₅₀ (μM) | | | IC₅₀ (μM) | | | IC₅₀ (μM) |
| | | MIC | IC₅₀ | Confidence | MIC | IC₅₀ | Confidence | MIC | IC₅₀ | Confidence |
| Organism with | Strain | (μM) | (μM) | interval 95% | (μM) | (μM) | interval 95% | (μM) | (μM) | interval 95% |
| One ltaS gene: | | | | | | | | | | |
| S. aureus-MRSA | USA300 | 3.1 | 1.3 | 1.1-1.4 | 6.2 | 1.7 | 1.4-2.0 | 25.0 | 8.4 | 6.6-10.7 |
| S. aureus-MSSA | RN4220 | 3.1 | 1.2 | 1.0-1.3 | 3.1 | 1.3 | 1.2-1.5 | 25.0 | 7.1 | 6.1-8.2 |
| Two ltaS genes: | | | | | | | | | | |
| E. faecalis-VRE | V583 | 50.0 | 18.5 | 23.8-14.3 | 100.0 | 55.6 | 42.9-72.2 | 100.0 | 36.7 | 30.8-43.7 |
| E. faecium-VRE | TX0016 | 50.0 | 7.3 | 4.7-11.3 | 100.0 | 14.6 | 8.9-23.9 | 100.0 | 19.4 | 14.4-26.2 |
| Four ltaS genes: | | | | | | | | | | |
| B. anthracis | Sterne | 1.6 | 1.0 | 0.9-1.1 | 3.1 | 1.5 | 1.1-2.0 | 25.0 | 11.1 | 8.4-14.8 |

¹⁾IC₅₀ values and 95%-confidence intervals were calculated by fitting data with variable slope sigmoidal dose-response function (GraphPad Prism 5)

### LTA Synthesis Inhibitors as Infectious Disease Therapeutics

Owing to the frequent use of antibiotics, members of the human microbiome continuously evolve drug resistance (DeLeo 2010). For MRSA, drug-resistance is associated with therapeutic failure and increased mortality of human infections (Klevens 2007, Klevens 2008). Glycopeptide (vancomycin)-resistance has transferred from enterococci to MRSA (Weigel 2003); the resulting VRSA strains are broadly antibiotic resistant and represent a global infectious threat (Tenover 2001, Li 2012). Daptomycin and linezolid have recently been licensed to address the threat of MRSA and VRSA infections (Arbeit 2004, Stevens 2002). Nevertheless, staphylococci quickly developed daptomycin- and linezolid-resistance, indicating that additional antibotics are needed to combat S. aureus infections (van Hal 2011). The crisis in antibiotic resistance applies also to other Gram-positive pathogens, including C. difficile, E. faecium, E. faecalis, S. epidermidis and Streptococcus pneumoniae (Neu 1992, Willems 2011).

LTA synthesis has been explored as a target for antibiotic therapy. Growth of S. aureus, B. anthracis, L. monocytogenes or B. subitilis cannot occur without polyglycerol-phosphate LTA synthesis and ltaS expression (Grundling 2007, Webb 2009, Schirner 2009, Wormann 2011). LtaS, the catalyst of LTA synthesis, harbors five transmembrane domains and a C-terminal catalytic domain that is found in bacteria but not in eukaryotes (Grundling 2007). The unique presence of LTA and LtaS in the envelope of bacterial species, the availability of the catalytic domain of LtaS on the bacterial surface and the requirement of LTA synthesis for bacterial growth and cell division fulfill key target features for the development of new antibiotics (Projan 2004). Compound 1771 was characterized as an LTA synthesis inhibitor, demonstrated its mechanism of action and ability to kill Gram-positive bacteria with polyglycerol-phosphate LTA. We were unable to isolate staphylococcal mutants with resistance against compound 1771, suggesting that LTA synthesis may display target attributes similar to peptidoglycan synthesis. Some Gram-positive bacteria, for example C. difficile and S. pneumoniae, synthesize LTA with distinct phosphate-polymer structures (Fischer 1997, Reid 2012), however their mechanisms of synthesis and possible inhibition by compound 1771 are not yet known. Future work must develop compound 1771 further to generate molecules that are stable in mammalian tissues and display antibiotic activity against many different bacteria. Such compounds may be useful therapeutics for human infectious diseases caused by drug-resistant Gram-positive bacteria.

### EXPERIMENTAL PROCEDURES

### High Throughput Screen

The NSRB library of 167,405 compounds was screened for molecules that inhibited the >90% growth of S. aureus RN4220 in Mueller-Hinton broth II supplemented with 0.005% Tween-80 in a 384 well format by measuring the optical density at 600 nm (Z′=0.72−0.84). A 98.9% pure preparation of 2-oxo-2-[(5-phenyl-1,3,4-oxadiazol-2-yl)amino]ethylnaphtho[2,1-b]furan-1-ylacetate compound 1771) was obtained from Enamine (catalog number T5526252).

### Growth Inhibition

Cultures of E. coli, S. aureus, C. perfringens, E. faecalis, E. faecium, B. anthracis and B. cereus were grown in the presence or absence of inhibitor in 96-well microplates at 37° C. for 18-22 hours and monitored by measuring the optical density at 600 nm.

### Inhibition of LTA Synthesis

Bacteria grown in the presence or absence of compound 1771 were lysed in a bead beater and cell extracts subjected to Coomassie-stained SDS-PAGE or immunoblotting using a monoclonal antibody to detect LTA/polyglycerol-phosphate and polyclonal antibodies for LtaS and SrtA.

eLtaS Inhibition

Size-exclusion High Performance Liquid Chromatography (HPLC) was performed with a BioBasic SEC300 column equilibrated in a 50 mM HEPES-KOH buffer, pH 7.5 containing 10 μM MnC12. The column was pre-equilibrated with 2 nmol 1-palmitoyl-2-[12-[7-nitro-2-1,3-benzoxadiazole-4-yl)amino]dodecanoyl]-sn-glycerol-3-[phospho-rac-(1-glycerol) (Avanti Polar Lipids). Purified eLtaS and SrtA (2 nmol) were injected and chromatograms recorded by measuring absorbance at 460 nm.

### Electron Microscopy

Samples were examined with a Fei Nova NanoSEM 200 scanning electron microscope (FEI Co., Hillsboro, Oreg., USA) operated with an acceleration voltage of 5 kV at a distance of 5 mm. Thin sectioned samples were viewed with a Tecnai F30 (Philips/FEI) transmission electron microscope (Field emission gun operating with a 300-kV accelerating voltage, using a magnification of 15,000-30,000×) and a high performance CCD camera with 4 k×4 k resolution.

### Animal Experiments

Animal experiments were performed in accordance with the institutional guidelines following experimental protocol review and approval by the Institutional Biosafety Committee (IBC) and the Institutional Animal Care and Use Committee (IACUC) at the University of Chicago. S. aureus Newman (1×108 CFU suspended in 100 μl PBS) was injected into the periorbital venous plexus of BALB/c mice (n=15) and animals were monitored for survival over 10 days. Animals received either two injections of inhibitor (32 mg/kg) or compound buffer (Mock) separated by 12 hour intervals prior to infection and an additional six doses post-infection.

Selecting for S. aureus Mutants with Increased Resistance to Compound 1771

Initial experiments sought to isolate antibiotic-resistant variants of S. aureus RNA4220 on LB agar plates with the Kirby-Bauer disk diffusion assay. Staphylococci (2.4×107 CFU) were inoculated per plate, which allowed for the isolation of streptomycin resistant variants (filter disk with 50 μmol streptomycin) but not for the isolation of compound 1771-resistant variants (filter disk with 200 μmol streptomycin). The mutation frequency for compound 1771-resistance was then analyzed with Mueller-Hinton agar plates containing a concentration gradient from 10 to 200 μM 1771. Large square plates (225 mm side length) were inoculated with 2×109 CFU S. aureus RN4220 per plate and incubated at either 37° C. or 42° C. Small, slow-growing colonies were observed only after 3-4 days of incubation. Three isolates were subjected to susceptibility testing against the 1771 inhibitor.

TABLE 5
Compound 1771 prevents cell cluster formation
in staphylococci and enterococci

Cell clusters/all cocci¹

| Microbe | mock | 1771 |
|---|---|---|
| S. aureus RN4220 | 53/636 | 3/387 |
| E. faecalis V583 | 34/390 | 2/496 |
| E. faecium TX0016 | 41/255 | 1/295 |

¹Bacterial growth in the presence of the LTA synthesis inhibitor (compound 1771) or in its absence (mock) was analyzed by scanning electron microscopy to reveal clusters of associated cocci (≧8 cells) compared to the sum of all individual cocci, diplococci or coccal clusters. Compound 1771 inhibition of S. aureus cluster formation was analyzed in multiple independent trials for statistical significance with the unpaired two-tailed Student's t-test (P = 0.0079).

TABLE 6
Treatment of Staphylococcus aureus, Enterococcus faecalis,
Enterococcus faecium and Bacillus anthracis with compound
1771 increases the diameter of the bacterial cell wall envelope

| | Cell envelope diameter (nm)¹ | | | Significance² |
|---|---|---|---|---|
| Microbe | mock | 1771 | Δ | (P value) |
| S. aureus | 31.2 (±0.6) | 38.8 (±0.9) | 7.5 (±1.1) | <0.0001 |
| RN4220 | | | | |
| E. faecalis | 23.8 (±0.4) | 31.6 (±0.6) | 7.8 (±0.7) | <0.0001 |
| V583 | | | | |
| E. faecium | 22.9 (±0.3) | 32.4 (±0.5) | 9.6 (±0.6) | <0.0001 |
| TX0016 | | | | |
| B. anthracis | 40.5 (±0.6) | 46.8 (±0.7) | 6.3 (±1.0) | <0.0001 |
| Sterne | | | | |

¹Bacteria were grown overnight on BHI agar in the absence (mock) or presence of the LTA synthesis inhibitor (1771) and were prepared for thin-section transmission electron microscopy to measure the envelope thickness, i.e. the diameter of the cell wall in nm. The mean of 124 measurements is indicated and the standard error of the means provided in parenthesis. The differential of the diameter (Δ) was calculated by subtracting the mean of the 1771 treated bacteria from that of mock-treated controls.
²Data were analyzed with the two-tailed Student's t-test for statistical significance and P values recorded.

TABLE 7
Resistance of S. aureus RN4220 parent and three colony isolates
from gradient plates with 10-200 μM compound 1771 (FIG. 11)

| | | | IC50 [μM] 95% |
|---|---|---|---|
| S. aureus strain | MIC [μM] | IC50 [μM] | confidence interval |
| parent | 12.5 | 7.3 | 5.9-9.1 |
| Isolate 1 | 18.8 | 8.7 | 8.3-9.2 |
| Isolate 2 | 18.8 | 9.2 | 8.8-9.6 |
| Isolate 3 | 18.8 | 12.2 | 11.8-12.6 |

S. aureus RN4220 and three isolates picked from selective agar plates containing compound 1771 were compared for their susceptibility to compound 1771 in growth inhibition experiments. Observed MIC and calculated IC50 are presented.

### REFERENCES

The following references, to the extent that they provide exemplary procedural or other details supplementary to those set forth herein, are specifically incorporated herein by reference.
U.S. Pat. No. 6,172,064
U.S. Pat. No. 6,191,150
U.S. Pat. No. 6,329,400
U.S. Pat. No. 7,723,349
U.S. patent application Ser. No. 10/518,110
U.S. patent application Ser. No. 12/160,862
U.S. patent application Ser. No. 12/327,313
U.S. patent application Ser. No. 12/605,118
U.S. Patent Ser. 60/534,501
U.S. Patent Ser. 60/512,016
U.S. Patent Ser. 60/515,352
U.S. Patent Ser. 61/453,648
U.S. Patent Ser. 61/411,765
U.S. Patent Ser. 61/511,032
PCT Appln. WO 03/051825
PCT Appln. WO 03/106381
Anand N & Davis B D (1960) Damage by streptomycin to the cell membrane of Escherichia coli. Nature 185:22-23.
Arbeit R D, Maki D, Tally F P, Campanaro E, & Eisenstein B I (2004) The safety and efficacy of daptomycin for the treatment of complicated skin and skin-structure infections. Clin. Infect. Dis. 38:1673-1681.
Baddiley J (1968) Technic acids in walls and the molecular structure of bacterial walls. Proc. R. Soc. London Ser. B 170:331-348.
Bhakdi and Tranum-Jensen, Microbiol. Rev., 55:733-751, 1991.
Brosnahan et al., Immunol., 182:2364-2373, 2009.
Bubeck Wardenburg and Schneewind, J. Exp. Med., 205:287-294, 2008.
Bubeck Wardenburg et al., Infect. Immun., 75:1040-1044, 2007.
Bubeck-Wardenburg et al., Nature Med., 13:1405-1407, 2007.
Callegan et al., Infect. Immun., 62:2478-2482, 1994.
Cleveland R F, et al. (1975) Inhibition of bacterial wall lysins by lipoteichoic acids and related compounds. Biochem. Biophys. Res. Commun. 67:1128-1135.
Coley J, Duckworth M, & Baddiley J (1972) The occurrence of lipoteichoic acids in the membranes of Gram-positive bacteria. J. Gen. Microbiol. 73:587-591.
Corrigan R M, Abbott J C, Burhenne H, Kaever V, & Grundling A (2011) c-di-AMP is a new second messenger in Staphylococcus aureus with a role in controlling cell size and envelope stress. PLoS Pathog 7(9):e1002217.
DeLeo F R, Otto M, Kreiswirth B N, & Chambers H F (2010) Community-associated meticillin-resistant Staphylococcus aureus. Lancet 375:1557-1568.
Dijkstra et al., Virchows Arch., 454:441-449, 2009.
Duckworth M, Archibald A R, & Baddiley J (1975) Lipoteichoic acid and lipoteichoic acid carrier in Staphylococcus aureus H. FEBS Lett. 53:176-179.
Dudek et al., Mol. Biol. Cell, 21(22):4042-4056, 2010.

Emorl and Gaynes, Clin. Microbiol. Rev., 6(4):428-42, 1993.
Fischer W (1990) Bacterial phosphoglycolipids and lipoteichoic acids. Handbook of lipid research, ed Hanahan D J (Plenum Press, New York), Vol 6, pp 123-234.
Fischer W (1997) Pneumococcal lipoteichoic and teichoic acid. Microb. Drug Resist. 3:309-325.
Funatsu G & Wittmann H G (1972) Ribosomal proteins. 33. Location of amino-acid replacements in protein S12 isolated from Escherichia coli mutants resistant to streptomycin. J. Mol. Biol. 68:547-550.
Garufi G, et al. (2012) Synthesis of lipoteichoic acids in Bacillus anthracis. J Bacteriol.
Garufi G, et al. (2012) Synthesis of lipoteichoic acids in Bacillus anthracis. J. Bacteria 194:4312-4321.
Goebel W F, Shedlovsky T, Lavin G I, & Adams M H (1943) The heterophile antigen of pneumococcus. J Biol Chem 148:1-15.
Gomez et al., EMBO J., 26:701-709, 2007.
Gonzalez et al., Cell Mol. Life Sci., 65:493-507, 2008.
Greenberg J W, Fischer W, & Joiner K A (1996) Influence of lipoteichoic acid structure on recognition by the macrophage scavenger receptor. Infect Immun 64(8):3318-3325.
Gründling A & Schneewind O (2007) Synthesis of glycerol phosphate lipoteichoic acid in Staphylococcus aureus. Proc. Nat. Acad. Sci. USA 104:8478-8483.
Gründling A & Schneewind O (2007) Genes required for glycolipid synthesis and lipoteichoic acid anchoring in Staphylococcus aureus. J. Bacteriol. 189:2521-2530.
Gründling A, Missiakas D M, & Schneewind O (2006) Staphylococcus aureus mutants with increased lysostaphin resistance. J. Bacteriol. 188:6286-6297.
Gumbiner, Cell, 84:345, 1996.
Harlow and Lane, In: Antibodies: A Laboratory Manual, Cold Spring Harbor Laboratory, 1988.
Hartmann et al., Hum. Mol. Genet., 11:2615-2624, 2002.
Hoettecke et al., Neurodegener. Dis., 7(4):232-238, 2010.
Hooper et al., Biochem. J., 321:265-279, 1997.
Hooper, FEBS Letters, 354:1-6, 1994.
Hoy et al., EMBO, 11:798, 2010.
Iacovache et al., Biochim Biophys Acta., 1778 (7-8): 1611-23, 2008.
Illek et al., Cell Physiol. Biochem., 22:57-68, 2008.
Inoshima et al., Nature Medicine, 2011 (IN PRESS)
Jones D, Metzger H J, Schatz A, & Waksman S A (1944) Control of Gram-negative bacteria in experimental animals by streptomycin. Science 100:103-105.
Jorasch P, Warnecke D C, Lindner B, Zahringer U, & Heinz E (2000) Novel processive and nonprocessive glycosyltransferases from Staphylococcus aureus and Arabidopsis thaliana synthesize glycolipids, glycophospholipids, glycosphingolipids, and glycosylsterols. Eur. J. Biochem. 267:3770-3783.
Jursch et al., Infect. Immun., 62:2249, 1994.
Karatsa-Dodgson M, Wörmann M E, & Gründling A (2010) In vitro analysis of the Staphylococcus aureus lipoteichoic acid synthase enzyme using fluorescently labeled lipids. J. Bacteriol. 192:5341-5349.
Karginov et al., Bioorg, Med, Chem., 15:5424, 2007.
Kennedy et al., J. Infect. Dis., 202(7):1050-1058, 2010.
Kim et al., Cell Host Microbe., 8(1):20-35, 2010a.
Kim et al., Vaccine, 28(38):6382-6392, 2010b.
Kiriukhin M Y, Debabov D V, Shinabarger D L, & Neuhaus F C (2001) Biosynthesis of the glycolipid anchor in lipoteichoic acid of Staphylococcus aureus RN4220: role of YpfP, the diglucosyldiacylglycerol synthase. J. Bacteriol. 183:3506-3514.
Klevens R M, et al. (2007) Invasive methicillin-resistant Staphylococcus aureus infections in the United States. JAMA 298:1763-1771.
Klevens R M, Edwards J R, Gaynes R P, & System NNIS (2008) The impact of antimicrobial-resistant, health care-associated infections on mortality in the United States. Clin. Infect. Dis. 47:927-930.
Koch H U, Haas R, & Fischer W (1984) The role of lipoteichoic acid biosynthesis in membrane lipid metabolism of growing Staphylococcus aureus. Eur. J. Biochem. 138:357-363.
Lambert P A, Hancock I C, & Baddiley J (1977) Occurrence and function of membrane teichoic acids. Biochim. Biophys. Acta. 472:1-12
Lemichez et al., Nat. Rev. Microbiol., 8(2):93-104, 2010.
Li M, et al. (2012) MRSA epidemic linked to a quickly spreading colonization and virulence determinant. Nat. Med. 18:816-819.
Lipinski C A, Lombardo F, Dominy B W, & Feeney P J (2001) Experimental and computational approaches to estimate solubility and permeability in drug discovery and development settings. Adv Drug Deliv Rev 46(1-3):3-26.
Lowy FD (1998) Staphylococcus aureus infections. New Engl. J. Med. 339:520-532.
Lu D, et al. (2009) Structure-based mechanism of lipoteichoic acid synthesis by Staphylococcus aureus LtaS. Proc Natl Acad Sci USA 106(5):1584-1589.
Ludwig et al., Comb. Chem. High Throughput Screen, 8:161-171, 2005.
Maretzky et al., Proc. Natl. Acad. Sci. USA, 102:9182-9187, 2005.
Maretzky et al., J. Invest. Dermatol., 128:1737-1746, 2008.
Marriott and Dockrell, Int. J. Biochem. Cell Biol., 38:1848-1854, 2006.
Martin et al., Infect. Immun., 79:1898-1904, 2011.
Matthay and Zemans, Annu. Rev. Pathol., 6:147, 2010.
Matthay and Zemans, Annu. Rev. Pathol., 28:147-163, 2011.
McAdow M, et al. (2011) Preventing Staphylococcus aureus sepsis through the inhibition of its agglutination in blood. PLoS Pathog 7(10):e1002307.
McGuire J M, Pittenger G E, Pittenger R C, & Stark W M (1955-1956) Vancomycin, a new antibiotic. I. Chemical and biologic properties. Antibiot. Annu. 3:606-611.
Menzies and Kernodle, Infect. Immun., 62:1843-1847, 1994.
Menzies and Kernodle, Infect. Immun, 64:1839-1841, 1996.
Morath S, von Aulock S, & Hartung T (2005) Structure/function relationships of lipoteichoic acids. J. Endotoxin Res. 11:348-356.
Murphy, Semin. Cell Dev. Biol., 20:138-145, 2009.
Neu H C (1992) The crisis in antibiotic resistance. Science 257:1064-1073.
Neuhaus F C & Baddiley J (2003) A continuum of anionic charge: structures and functions of D-alanyl-teichoic acids in gram-positive bacteria. Microbiol. Mol. Biol. Rev. 67:686-723.
O'Callaghan et al., Infect. Immun., 65:1571-1578, 1997.
Oku Y, et al. (2009) Pleiotropic roles of polyglycerolphosphate synthase of lipoteichoic acid in growth of Staphylococcus aureus cells. J. Bacteriol. 191:141-151.
Ong and Leung, Immun. Allergy Clincis of NA, 30:309-321, 2010.
Patel et al., Infect. Immun., 55:3103-3110, 1987.
Perl et al., Proc. Natl. Acad. Sci. USA, 99:10482-10487, 2002a.
Perl et al., Transgenic Res., 11:21-29, 2002b.
Pochetuhen et al., Am. J. Pathol., 171(2):428-437, 2007.
Powers et al., J. Infect. Diseases, 2011 (IN PRESS)
Projan S J & Shlaes D M (2004) Antibacterial drug discovery: is it all downhill from here? Clin. Microbiol. Infect. 10:18-22.
Ragle and Bubeck-Wardenburg, Infect. Immun., 77:2712-2718, 2009.
Ragle et al., Antimicrob. Agents Chemother., 54:298, 2010.
Reichmann N T & Gründling A (2011) Location, synthesis and function of glycolipids and polyglycerolphosphate lipoteichoic acid in Gram-positive bacteria of the phylum Firmicutes. FEMS Microbiol. Lett. 319:97-105.
Reid C W, et al. (2012) Structural characterization of surface glycans from Clostridium difficile. Carbohydr. Res. 354:65-73.
Reiss and Saftig, Semin. Cell Dev. Biol., 20:126-137, 2009.
Rice et al., Am. J. Physiol. Lung Cell Mol. Physiol., 283:L256-264, 2002.
Rishton G M (2003) Nonleadlikeness and leadlikeness in biochemical screening. Drug Discov Today 8(2):86-96.
Rubins et al., J. Clin. Invest., 95:142-150, 1995.
Schirner K, Marles-Wright J, Lewis R J, & Errington J (2009) Distinct and essential morphogenetic functions for wall- and lipo-teichoic acids in Bacillus subtilis. EMBO J. 28:830-842.
Schulte et al., Cell Death and Differentiation, 14:1040-1049, 2007.
Schulz et al., Circ. Res., 102(10):1192-1201, 2008.
Seals and Courtneidge, Genes Dev., 17:7-30, 2003.
Seidler J, McGovern S L, Doman T N, & Shoichet B K (2003) Identification and prediction of promiscuous aggregating inhibitors among known drugs. J Med Chem 46(21):4477-4486.
Shapiro and Weis, Cold Spring Harb. Perspect. Biol., 1:a003053, 2009.
Song et al., Science, 274:1859-1866, 1996.
Steinhusen et al., J. Biol. Chem., 276:4972-4980, 2001.
Stevens D H, et al. (2002) Linezolid versus vancomycin for the treatment of methicillin-resistant Staphylococcus aureus infections. Clin. Infect. Dis. 34:1481-1490.
Tenover F C, Biddle J W, & Lancaster M V (2001) Increasing resistance to vancomycin and other glycopeptides in Staphylococcus aureus. Emerg. Infect. Dis. 7:327-332.
Theilacker C, et al. (2012) Protection against Staphylococcus aureus by antibody to the polyglycerolphosphate backbone of heterologous lipoteichoic acid. J Infect Dis 205(7):1076-1085.
Tian et al., Int. Immunol., 20:1181-1187, 2008.
Tomita and Kamio, Biosci. Biotechnol. Biochem., 61:565-572, 1997.
Ton-That H, Liu G, Mazmanian S K, Faull K F, & Schneewind O (1999) Purification and characterization of sortase, the transpeptidase that cleaves surface proteins of Staphylococcus aureus at the LPXTG motif. Proc. Natl. Acad. Sci. USA 96:12424-12429.
Ton-That H, Mazmanian S K, Alksne L, & Schneewind O (2002) Anchoring of surface proteins to the cell wall of Staphylococcus aureus. Cysteine 184 and histidine 120 of sortase form a thiolate-imidazolium ion pair for catalysis. J Biol Chem 277(9):7447-7452.
Tweten, Infection and Immunity, 73 (10): 6199-6209, 2005.
van Hal S J & Paterson D L (2011) New Gram-positive antibiotics: better than vancomycin? Curr. Opin. Infect. Dis. 24:515-520.
Vasioukhin et al., Proc. Natl. Acad. Sci. USA, 96:8551-8556, 1999.
Walker and Bayley, J. Biol. Chem., 270:23065, 1995.
Walsh C T (2000) Molecular mechanisms that confer antibacterial drug resistance. Nature 406:775-781.
Weigel L M, et al. (2003) Genetic analysis of a high-level vancomycin-resistant isolate of Staphylococcus aureus. Science 302:1569-1571.
Wilke and Bubeck-Wardenburg, Proc. Natl. Acad. Sci. USA, 107(30):13473-13478, 2010.
Webb A J, Karatsa-Dodgson M, & Gründling A (2009) Two-enzyme systems for glycolipid and polyglycerolphosphate lipoteichoic acid synthesis in Listeria monocytogenes. Mol. Microbiol. 74:299-314.
Willems R J, Hanage W P, Bessen D E, & Feil E J (2011) Population biology of Gram-positive pathogens: high-risk clones for dissemination of antibiotic resistance. FEMS Microbiol. Rev. 35:872-900.
Wörmann M E, Corrigan R M, Simpson P J, Matthews S J, & Gründling A (2011) Enzymatic activities and functional interdependencies of Bacillus subtilis lipoteichoic acid synthesis enzymes. Mol. Microbiol. 79:566-583.
Wu et al., Proc. Natl. Acad. Sci. USA, 95:14979, 1998.
Yamakawa J, et al. (2012) Heterogeneously vancomycin-intermediate Staphylococcus aureus (hVISA) emerged before the clinical introduction of vancomycin in Japan: a retrospective study. J. Infect. Chemother. 18:406-409.
Zhang J H, Chung T D, & Oldenburg K R (1999) A Simple Statistical Parameter for Use in Evaluation and Validation of High Throughput Screening Assays. J Biomol Screen 4(2):67-73.

1. A method of inhibiting a Gram-positive bacteria infection in a patient comprising administering to the patient a composition comprising a lipoteichoic acid synthase (LtaS) inhibitor that comprises an oxadiazole ring.

2. The method of claim 1, wherein the Gram-positive bacteria is Staphylococcus aureus.

3-4. (canceled)

5. The method of claim 1. wherein the Gram-positive bacteria is Bacillus anthracis.

6. The method of claim 1, wherein the Gram-positive bacteria is Enterococcus faecium.

7-10. (canceled)

11. The method of claim 1, wherein the LtaS inhibitor comprises a hydrophobic double-ring structure.

12. The method of claim 11, wherein the hydrophobic double-ring structure comprises naphthofuran.

13. The method of claim 1, wherein the LtaS inhibitor has the following structure: 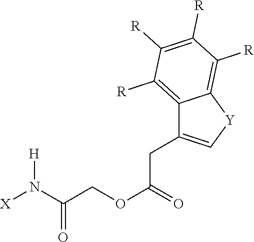
wherein X is an aryl group; Y is O or NH; and each R is independently H, alkyl, aryl, or heteroaryl or two adjacent R groups form a saturated or unsaturated carbocyclic or heterocyclic ring, and wherein at least one R is not H.

14. The method of claim 13, wherein the LtaS inhibitor is 2-oxo-2-(5-phenyl-1,3,4-oxadiazol-2-ylamino)ethyl2-naphtho[2,1-b]furan-1-ylacetate or a salt thereof.

15. The method of claim 1, wherein the composition is administered orally, topically, nasally, intravascularly, intraperiotoneally, intrathecally, intratracheally, by inhalation or instillation.

16. (canceled)

17. The method of claim 1, further comprising administering a second anti-microbial treatment.

18. (canceled)

19. The method of claim 1, wherein the patient has been determined to have a Gram-positive bacteria infection.

20. The method of claim 1, further comprising identifying the patient as having a Gram-positive bacteria infection.

21-23. (canceled)

24. The method of claim 19, wherein the patient is administered the LtaS inhibitor within 1 week of being determined to have a Gram-positive bacteria infection.

25. The method of claim 1, wherein the patient is at risk of Gram-positive bacteria infection.

26. The method of claim 25, wherein the patient is immune deficient, is immunocompromised, is hospitalized, is undergoing an invasive medical procedure, is infected with influenza virus or is on a respirator.

27. The method of claim 1, wherein the patient has pneumonia, sepsis, corneal infection, a skin infection, an infection of the central nervous system, or toxic shock syndrome.

28. The method of claim 1, wherein the patient exhibits a skin abscess, a boil, or a furuncle.

29. (canceled)

30. A method for inhibiting lipoteichoic acid synthesis in a Gram positive bacteria comprising administering to the bacteria a lipoteichoic acid synthase (LtaS) inhibitor that comprises an oxadiazole ring.

31-59. (canceled)

60. A method of treating a subject having or at risk of developing a Gram-positive bacteria infection comprising administering an effective amount of a pharmaceutically acceptable composition comprising an LtaS inhibitor having an oxadiazole ring and a hydrophobic double-ring structure, or salt thereof.

61. The method of claim 60, wherein the compound is 2-oxo-2-(5-phenyl-1,3,4-oxadiazol-2-ylamino)ethyl2-naphtho[2,1-b]furan-1-ylacetate, or a salt thereof.
